(19) United States
(12) Patent Application Publication
(10) Pub. No.: US 2008/0293698 A1
(43) Pub. Date: Nov. 27, 2008

(54) Methods and Compositions for Treating Arg

(76) Inventor: Joseph Johnson, Springfield, TN (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/913,454

(22) PCT Filed: May 12, 2006
(86) PCT No.: PCT/US06/18460

§ 371 Date: May 6, 2008

Related U.S. Application Data
(60) Provisional application No. 60/681,248, filed on May 16, 2005.
(60) Provisional application No. 60/720,508, filed on Sep. 26, 2005.
(60) Provisional application No. 60/723,325, filed on Oct. 4, 2005.
(60) Provisional application No. 60/749,129, filed on Dec. 9, 2005.

(51) Int. Cl. A61K 31/554; A61K 31/5513; A61K 31/437; A61P 43/00

(52) U.S. Cl. 514/220; 514/221; 514/300

(57) ABSTRACT

The invention provides dilute and concentrated, aqueous, pharmaceutical compositions comprising gamma-hydroxybutyric acid or pharmaceutically acceptable salts thereof; gamma-butyryl lactone; 1,4-butanediol; 4-hydroxyl pentanoic acid or pharmaceutically acceptable salts thereof; 4-hydroxyl pentanoic acid lactone, or combinations thereof, and a coloring agent and/or flavoring agent that is useful in preventing sexual assault or date rape. Methods of treating conditions responsive to the administration of gamma-hydroxyl butyric acid and/or its pharmaceutically acceptable salts; gamma-butyryl lactone; 1,4-butanediol; 4-hydroxyl pentanoic acid and/or its pharmaceutically acceptable salts; and 4-hydroxyl pentanoic acid lactone are also described. The invention provides methods for treating patients with acquired resistance to GABAnergic agents.

### BACKGROUND

Gamma-hydroxyl butyric acid (GHB) is an endogenous compound with hypnotic properties that is found in many human body tissues. GHB is present, for example, in the mammalian brain and other tissues. In brain the highest GHB concentration is found in the hypothalamus and basal ganglia and GHB is postulated to function as a neurotransmitter. The neuropharmacologic effects of GHB include increases in brain acetylcholine, increases in brain dopamine, inhibition of GABA-ketoglutarate transaminase and depression of glucose utilization but not oxygen consumption in the brain. GHB is converted to succinate and then metabolized via the Krebs cycle. Clinical trials have shown that GHB increases delta sleep and improves the continuity of sleep.

GHB has several clinical applications other than narcolepsy and sleep disorders. GHB has been reported to reduce alcohol craving, the number of daily drinks consumed, and the symptoms of alcohol withdrawal in patients. GHB has been used to decrease the symptoms of opiate withdrawal, including both heroin and methadone withdrawal. It has analgesic effects that make it suitable as a pain reliever. Intravenous administration of GHB has been reported to reduce intracranial pressure in patients. Also, administration of GHB was reported to increase growth hormone levels in patients.

GHB is also colorless and odorless. It has typically been administered in clinical trials as an oral solution. GHB treatment substantially reduces the signs and symptoms of narcolepsy, i.e. daytime sleepiness, cataplexy, sleep paralysis and hypnagogic hallucinations. In addition, GHB increases total sleep time and REM sleep, and it decreases REM latency and improves general anesthesia.

GHB has risks beyond unintended disinhibition, however. The drug can cause unconsciousness, respiratory depression, bradycardia, nausea, vomiting, seizures and coma. The severity of symptoms and the duration of action are dose dependent and can be affected by the presence of other CNS depressants.

1,4-butanediol and gamma-butyryl lactone (GBL) have similar physiologic effects as gamma-hydroxyl butyrate (GHB). 1,4-butanediol and GBL are clear, colorless liquids. GHB; gamma-butyryl lactone; and 1,4-butanediol may be aggravators of central and obstructive sleep apnea in some patients. 4-hydroxyl pentanoic acid, pharmaceutically acceptable salts thereof (4-HPA), and 4-hydroxyl pentanoic acid lactone (4-HPA lactone) have similar relaxant effects as GHB but are less toxic.

In the brain, 1,4-butanediol is converted to gamma-hydroxylbutyryl aldehyde by alcohol dehydrogenase. Gamma-hydroxylbutyryl aldehyde is converted into gamma-hydroxyl butyrate (GHB). GHB is convertible to gamma-aminobutyric acid (GABA) through enzymatic changes. Gamma-butyryl lactone, when ingested orally, is transformed to GHB via peripheral lactonases. In the body, GHB is transformed to Krebs cycle intermediates and succinate, which enters the Krebs cycle. When ingested orally, gamma-aminobutyric acid has little biological effect. It is poorly absorbed and it does not cross the blood-brain barrier. In contrast, GHB is readily absorbed when ingested orally and crosses the blood brain barrier.

GHB; GBL; and 1,4-butanediol are central nervous system (CNS) depressants at low doses, and have the curious effects of reducing anxiety and producing euphoria and relaxation, sedating the recipient.

Because of these properties, the drugs have been abused through surreptitious administration to unsuspecting users in a variety of settings, including college parties and bars in the United States. The drugs have thus become known as agents of sexual assault, used to disable persons who have unknowingly ingested the drug in a product they otherwise intended to consume.

GHB; GBL, and 1,4-butanediol have risks beyond unintended disinhibition, however. The drugs can cause unconsciousness, respiratory depression, bradycardia, nausea, vomiting, seizures and coma. The severity of symptoms and the duration of action are dose dependent and can be affected by the presence of other central nervous system depressants.

GHB; GBL; and 1,4-butanediol have steep dose-response curves. A 1-gram dose of GHB for a 150-pound person provides a low degree of effect, causing a sense of euphoria and loss of inhibitions. However, a 2.5-gram dose to the same individual can lead to coma. A typical dose of sodium gamma-hydroxybutyrate for the treatment of narcolepsy is 3 to 4.5 grams.

Since GHB; 1,4-butanediol; GBL; 4-HPA solutions; and 4-HPA lactone are clear, colorless, and not strong tasting when diluted, they can be surreptitiously administered to an un-suspecting person. The relationship between 4-hydroxyl pentanoic acid, salts thereof, and 4-hydroxyl pentanoic acid lactone has been documented and these agents are expected to be therapeutic for similar conditions treatable with GHB, although the substances have different properties as GHB and individual metabolism varies between patients. 4-hydroxyl pentanoic acid, salts thereof, and 4-hydroxyl pentanoic acid lactone are less toxic than GHB. 4-hydroxyl pentanoic acid lactone has been reported to bind the GHB receptor (United States patent application 20020132846). Previous solid or semi-solid formulations of salts of gamma-hydroxyl butyric acid have not been intended to warn un-suspecting persons that unintended substances have been added to food or beverage, and therefore have not included colors which are more distinctive than typical pills or capsules, and have not included unusual or distinctive tastes for a pharmaceutical agent. Solid state formulations of GHB are known but have not been intended to prevent sexual assault. For example, Gessa in U.S. Pat. No. 4,983,632 discloses an effervescent tablet of the sodium salt of GHB (NaGHB) in example 3 but only for the purposes of palatability and does not include in any particularly distinctive taste or color. Gessa(ibid.) discloses an effervescent sachet in example 4. However, examination of the ingredients, which are NaGHB, lyophilized orange juice, orange flavoring, sodium saccharin, and saccharose, reveals that the composition is not effervescent, as it contains only small amounts of appropriate acidifying agents such as citric acid in the dried orange juice and no gas-emitting substance. Gessa was referring in example 4 to a sparkling taste and not effervescence.

Orphan Medical in U.S. Pat. No. 6,472,431 makes vague references to GHB in tablets, pills, and capsules including excipients. Orphan Medical(ibid.) in U.S. Pat. No. 6,472,431 envisioned easy and undetectable or hardly detectable addition of GHB to food or beverage and stated(ibid.) “ . . . oral compositions . . . may be compressed into tablets . . . to be admixed with an aqueous medium for oral or injectable formulations, or they may be incorporated directly with food (i.e. beverage) of the diet.” As illustrated by this quote, Orphan Medical was not concerned with date rape. Consequently, there is a need to formulate the substances in such a way that cannot be administered to an unsuspecting person.

Acquired resistance to GABAnergic agents (ARG) has been a plague amongst humanity for centuries. ARG is associated with unrestful sleep and alpha-wave intrusion into sleep. Because ARG may be associated with other sleep disorders, although it usually occurs in isolation, a discussion of other sleep disorders is warranted. Obstructive sleep apnea (OSA) is a disease in which the pharynx, usually at the level of the tongue base or soft palate, intermittently collapses during sleep, resulting in attempted inspiration against a closed airway. Increased negative pressure is generated in the chest cavity during these inspiratory efforts. The episodes of increased negative pressure are brief and do not span the length of the apnea. Negative intrathoracic pressures as high as negative about 80-90 cm water have been documented. Normally, negative intrathoracic pressure does not dip below negative 8 cm water. Apneas typically last from 10 to 45 seconds but can last over two minutes. A low number of apneas is 5 to 20 per minute of sleep. 21 to 40 apneas may be considered moderate. Over forty apneas is considered severe. However, as few as 6 or 7 apneas per hour can cause significant symptomotology. The degree to which OSA causes fatigue is largely based on the degree to which stages 3 and 4 sleep (slow wave sleep) are reduced. Typically, a young adult has 20% stages 3 and 4 sleep. The amount of stages 3 and 4 sleep decreases with age. Patients below the age of 60 years who are chronically getting 0% stages 3 and 4 sleep due to sleep disorders are typically tired. Prolongation of sleep time with medications may be used to compensate for decreased stages 3 and 4 sleep. Treatments which increase stages 3 and 4 sleep sometimes decrease fatigue, although their are many polysomnographic criteria, and reliance on polysomnographic criteria is less important than questioning the patient about subjective improvements in symptoms such as fatigue. In fact, little to no increase in stages 3/4 sleep does not mean that a treatment is ineffective; on the contrary, the treatment may nonetheless be very effective. OSA has been associated with altered cerebral hemodynamics (Sleep apnea and autonomic cerebrovascular dysfunction; Loeppky J A, Voyles W F, Eldridge M W, Sikes C W; Sleep 1987 February; 10(1):25-34; Intracranial hemodynamics in sleep apnea; Fischer, Chauduri B A, Taorima M, Aktar B; Chest 1992 November; 102(5):1402-1406, 1992; Cerebral hemodynamics in obstructive sleep apnea; Siebler M, Nachtmann A; Chest 1993 April 103(4):1118-1119; Impairment of cerebral perfusion during obstructive sleep apnea; Balfors E M, Franklin K A; American Journal of Respiratory and Critical Care Medicine 1994(150):1587-1591; Changes in cerebral oxygenation and hemodynamics during obstructive sleep apneas; Hayakawa T, Terashima M, Kayukawa Y, Ohta T, Okada T; Chest 1996 April; 109(4):916-21; Sleep apnea syndrome and cerebral hemodynamics; Hajak G, Klingenhoefer J, Schulz-Varzegi M, sander D, Ruether E; Chest September 1996 110(3): 1670-679). Whether this is due to oxygen desaturations during sleep or related to episodes of increased negative intrathoracic pressure is unclear. The most common symptom of OSA is fatigue. Other symptoms include poor attention span, attention deficit, symptoms vaguely reminiscent of bipolar disease, increased desire to sleep during the day, tendency to nap frequently or doze unintentionally, and insomnia. Often a patient has increased desire to sleep during the day yet has insomnia at night.

Chronic untreated OSA has been associated with congestive heart failure and cardiomyopathy, such as dilated cardiomyopathy. The negative intrathoracic pressures generated by some patients are sufficient to suck blood from the abdomen into the low pressure side of the heart, the right ventricle, thus dilating it and compressing the left ventricle briefly. Abnormalities in atrial natriuretic factor, a cardiac hormone, may occur. Also, such patients typically have oxygen desaurations during sleep. Cardiomyopathy associated with OSA may be referred to as negative pressure cardiomyopathy.

Narcolepsy is a disease that is also characterized by fatigue. Most narcolepsy patients have an increased desire to sleep during the day. Other symptoms of narcolepsy include hallucinations just after awakening or just prior to sleep (hypnagogic and hypnapompic hallucinations). Such hallucinations are typically people standing around the bed, geometric patterns, brightly colored objects and lights, or auditory hallucinations. Pleasant or vindictive voices may be heard. Often the patient is aware that the hallucinations are not real and can think lucidly while viewing or hearing them. Sleep paralysis is associated with narcolepsy and occasionally with OSA. During sleep paralysis, the patient wakes up and is briefly paralyzed although may occasionally be able to open the eyes or attempt to mumble words. The episodes are brief but are at times perceived by the patient to last for hours. Hypnogogic hallucinations may co-occur with sleep paralysis. Vivid or surreal dreams are common in narcolepsy. Patients who fall asleep during the day and have vivid dreams during short naps (less than 30 minutes) often have OSA or narcolepsy. Some narcolepsy patients experience strobe dreaming, in which on certain nights they have numerous vivid dreams interrupted by frequent awakenings. After awakening for just a few seconds, the patients then rapidly falls back asleep into the dream world. A minority of narcolepsy patients experience cataplexy, which are episodes of difficulty initiating movement in the body while awake, or difficulty maintaining muscle tone. Occasionally the patient goes to sleep after an episode of cataplexy. Cataplexy can occur without inciting factors or may be triggered by laughter or excitement.

The pathophysiology of narcolepsy is unknown. Hypothalamic dysfunction has been implicated. A subset of patients have very low levels of hypocretin-1 in the cerebral spinal fluid.

Postulation of the following would go far in explaining the pathophysiology of narcolepsy: there may be one or more central sleep debt monitors in or near the hypothalamus. As sleep debt increases during the day, the central sleep debt monitors eventually sensitize neurons to the sleep-inducing effects of a variety of compounds. If the sleep debt monitor or sensitization to sleep debt become dysfunctional, the patient develops some degree of narcolepsy. The patient has chronic sleep debt because the sleep debt monitor/sleep sensitization system cannot ensure proper triggering and maintenance of sleep. Thus, the peripheral brain regulates sleep. Hypothalamically-driven sleep is preferable to peripherally-driven sleep.

Classic cases of narcolepsy occur in roughly 1 in 2000 patients. These cases of narcolepsy may be referred to as narcolepsy major. However, mild to moderate cases of dysfunction of sleep debt monitoring or sensitization to sleep debt (narcolepsy minor) probably occur in roughly 1 in 300 patients. Narcolepsy major and narcolepsy minor are treated similarly involving one or more GABAnergic agents prior to sleep.

Idiopathic hypersomnia (IHS) is a condition characterized by severe fatigue and sleepiness and unrestful sleep. Idiopathic hypersomnia is less common than narcolepsy major. However, recent studies have shown that a subset of patients with IHS have low levels of hypocretin-1, indicating that some patients in fact have narcolepsy. Whether a subset of IHS patients have hypersensitivity to endogenous GABAnergic agents is unknown.

The multiple sleep latency test (MSLT) is roughly 60 percent sensitive in detecting narcolepsy major. Patients who test positive exhibit rapid entry into REM sleep when given nap opportunities. However, some patients with narcolepsy major have normal sleep latencies. Patients with IHS tend to enter non-REM sleep quickly on MSLT. However, some narcolepsy patients show the same pattern, and in practice it can be difficult to distinguish narcolepsy major or narcolepsy minor from IHS.

Neither narcolepsy major, narcolepsy minor, nor idiopathic hypersomnia segregate independently from obstructive sleep apnea. A higher proportion than expected of narcolepsy/IHS patients have osA. This is likely due the altered cerebral hemodynamics associated with OSA. Abnormal blood flow to the postulated central sleep debt monitors may trigger some degree of narcolepsy. Patients who have underlying narcolepsy minor may develop narcolepsy major upon developing OSA.

The most common treatment for OSA is continuous positive airway pressure (CPAP). This is accomplished by delivering positive air pressure, usually by a nasal mask, to the patient during sleep. In response the pressure, the tongue either is passively made to abut against the palate or reflexively contracts upward, and the pharynx is pneumatically splinted to prevent collapse of the airway. The lowest pressure to eliminate apneas is used, typically 5 cm to 18 cm water pressure. Although chronic use of CPAP is highly beneficial to most patients with coexisting OSA and narcolepsy, the pressures of CPAP are significant and on occasion chronic use of CPAP can aggravate narcolepsy or cause sleep pattern similar to IHS in which sleep becomes unrestful and the patient has chronic desire or tendency to sleep. This condition may be referred to as CPAP-induced hypersomnia. Again, however, it is noted that CPAP is beneficial for the majority of patients.

Restless legs syndrome (RLS) is characterized by the desire to move the legs while lying flat and which is relieved by standing or walking. Often motor restlessness occurs and is increased prior to sleep. A minority of patients with RLS report odd sensations in the legs while lying flat such as crawling, quivering, painful, cramping, or electrical sensations when lying flat. RLS is associated with periodic limb movement disorder, a condition in which the legs move in a stereotypic manner at intervals during sleep. Periodic limb movement disorder (PLMD) often occurs on some nights and not others for a given patient. However, such patients often have insomnia on a nightly basis. It is expected that some patients with insomnia, anxiety, attention deficit, hyperactivity, or motor hyperactivity have subclinical restless legs syndrome or subclinical periodic limb movement disorder. Subclinical restless legs syndrome is manifested by subtle restlessness or symptoms which do not meet current criteria for restless legs syndrome. Subclinical periodic limb movement disorder is manifested by non-specific polysomnographic abnormalities such as frequent brief awakenings or arousals not associated with stereotypic leg movements. The treatment of RLS and subclinical RLS is expected to be the same, as is the treatment of PLMD and subclinical PLMD.

REM sleep behavior disorder is a condition in which the patient hits, fights, or screams during REM sleep. The condition typically occurs in men in the sixth or seventh decade of life, many of whom have Parkinson's disease. However, REM sleep behavior disorder can occasionally occur in younger patients who do not have Parkinson's disease. The most common treatment of REM sleep behavior disorder is clonazepam 0.5-2 mg prior to sleep, although younger patients may be tried on higher doses.

ARG typically falls into one of two categories although some cases of ARG do not fall into either category. ARG type I is typified by chronic fatigue and has hitherto been referred to as chronic fatigue syndrome. ARG type II is typified by fatigue and myalgias or arthralgias and has hitherto been referred to as fibromyalgia, fibrositis, or neuromyasthenia. Some patients with ARG have psychiatric symptoms such as depression, attention deficit, hyperactivity, symptoms reminiscent of bipolar disorder, anxiety disorders, and obsessive compulsiveness but minimal fatigue and cannot be classified as ARG type I or ARG type II.

The present invention relates generally to treating ARG. Some patients with ARG cannot be categorized as ARG type I or ARG type II but may be treated with the methods described herein. ARG type I and ARG type II are distinct disease entities sometimes occurring in isolation.

### FIELD OF THE INVENTION REGARDING TREATMENT OF ARG, ARG TYPE I AND ARG TYPE II

Although it is clear to the inventor that ARG type I and ARG type II are diseases, there is no general agreement that ARG type I or ARG type II are diseases in the medical community. Some physicians continue to refer to ARG type II as “psychogenic rheumatism,” a totally inappropriate term which suggests the patient is not ill. Many physicians refuse to acknowledge ARG type I or ARG type II at all.

Acquired resistance to GABAminergic agents (ARG) is a disease the symptoms of which may be varied including chronic, severe fatigue (the feeling of tiredness), myalgias (muscle aching), arthralgias (joint aching), difficulty concentrating (brain fog), and unrestful sleep. ARG has hitherto been thought to be a mysterious syndrome. Others have postulated dysfunction of the hypothalamus as the etiology. The association between ARG and intrusion of waking brain wave patterns into sleep has been documented and is referred to as alpha-wave intrusion. ARG has heretofore been referred to as fibromyalgia, fibrositis, neuromyasthenia, chronic fatigue syndrome, and chronic fatigue and immune dysfunction syndrome. Patients with myalgias and arthralgias are typically diagnosed with fibromyalgia, whereas patients with unexplained fatigue are diagnosed with chronic fatigue syndrome. A minority of patients have intermittent or chronic infections such as candida or cold sores or intermittently swollen lymph nodes. ARG effects roughly six million patients in the United States.

The typical age of onset of ARG is in the third through sixth decade of life. After the sixth and seventh decades the disease frequently wanes. The majority of sufferers are women.

Gamma-aminobutyric acid (GABA) is the primary inhibitory neurotransmitter of the brain. Cumulative evidence suggests that resistance to GABA and its cogeners are the etiology of chronic fatigue syndrome and fibromyalgia. The GABA receptor is a large protein with multiple subunits. In effect the GABA receptor functions as many different receptors. Numerous subunits of the GABA-A, GABA-B, and GABA-C receptors have been identified.

GABAnergic agents, which are also referred to as GABAminergic agents, are substances which bind the GABA receptor or augment the actions of GABA and its cogeners.

The function of sleep in adult humans may be to decrease the amount of energy at the neuronal cell surface membrane, to decrease the number of synapses in use, or to decrease the amount of electrical bleed between neighboring synapses. The most common symptom of most sleep disorders is fatigue. Fascinatingly, many sleep specialists refuse to use either the words “fatigued” or “tired” and refuse to design a scale for fatigue. Such an attitude makes the study of sleep disorders and disorders of sleep/wake impossible. Fatigue as the feeling of tiredness should be distinguished from muscle fatigue, which is weakness due to exertion or weakness due to repetitive muscle contraction. Although some patients with sleep disorders and ARG complain of lack of energy, they may in fact have an excess of energy on the neuronal cell surface membrane, leading to the symptom of fatigue, which may be disabling, interfering with concentration and activities of daily living. Much of intracellular energy is generated in the mitochondria. Whether patients with ARG have a deficiency in neuronal mitochondrial energy is unknown.

Improvement in subjective symptoms such as decrease in fatigue or increased feeling of well being are more important than improvement in polysomnographic parameters in treating patients with ARG.

Patients with demyelinating diseases such as multiple sclerosis have decreased flow of electricity along the central nervous system neuronal cell surface membranes and also complain of severe fatigue. Thus, having either increased or decreased energy at the neuronal surface membrane leads to fatigue.

GHB has typically been administered in clinical trials as an oral solution. GHB treatment substantially reduces the signs and symptoms of narcolepsy, i.e. daytime sleepiness, cataplexy, sleep paralysis and hypnagogic hallucinations. GHB may be an aggravator of central and obstructive sleep apnea in some patients.

Three documents are particularly relevant when discussing ARG and GHB. They are: 1) Document A, which is U.S. Pat. No. 5,990,162; inventor Martin B. Scharf; 2) Document B, which is Martin B. Scharf et al.: {Effect of gamma-hydroxybutyrate on pain, fatigue, and the alpha sleep anomaly in patients with fibromyalgia. Preliminary report.; Scharf M B, Hauck M, Stover R, McDannold M, Berkowitz D; Journal of Rheumatology. 1998 October; 25(10): 1986-1990.}; 3) Document C, which is Martin B. Scharf et al.: {The effects of sodium oxybate on clinical symptoms and sleep patterns in patients with fibromyalgia; Scharf M B, Baumann M, Berkowitz D V, Journal of Rheumatology. 2003 May; 30(5):1070-4}.

The sodium salt of GHB has been used at times in inappropriately low doses in the treatment of ARG type I and ARG type II and has led to false conclusions regarding the treatment of ARG type I and ARG type II. In document B Scharf et al. described what they called the baffling symptoms of fibromyalgia (ARG type II) and administered 2.25 grams of the sodium salt of GHB prior to sleep and again four hours later. In some patients the dosage of GHB was “adjusted slightly upwards.” GABAnergic agents including zolpidem, clonazepam, and alprazolam were withdrawn from the medication regimens of some patients prior to institution of treatment with GHB. An additional sedating compound which was unspecified was used in some patients.

In document C Scharf et al. administered 3 grams of the sodium salt of GHB prior to sleep and then a second 3 gram dose 4 hours later. No further GABAminergic agents were instituted other than GHB. Again, the failure to treat with adequate dosages and sufficient varieties of GABAminergic agents was based on the failure to comprehend the pathophysiology of the disease.

In documents A, B, and C Scharf or Scharf et al. were unaware that they were treating ARG and therefore use incorrect terminology. The low doses of GHB used and discontinuation of useful GABAnergic agents rather than an increase of dosage of GABAnergic agents indicate an incorrect understanding of ARG and led to treatment recommendations which do not adequately treat many patients with ARG.

The statements of Scharf or Scharf et al. in documents A, B, and C are contradictory. After patenting a method of treating both chronic fatigue syndrome (ARG type I) and fibromyalgia (ARG type II) in document A, Scharf then reversed his position and stated that his treatment is ineffective for chronic fatigue syndrome (ARG type I) in document C. Scharf et al. in document C state in regards to Scharf et al. in document B “In our pilot open label trial, sodium oxybate had no effect in patients diagnosed with chronic fatigue syndrome—a condition with symptoms overlapping with FM.” Scharf at times refers to GHB as sodium oxybate. FM refers to fibromyalgia. Scharf et al. did not comprehend that their treatment failure was due to insufficient dosages of GHB and the failure to use any other GABAnergic agent.

Scharf et al. in document C state the following regarding fibromyalgia and chronic fatigue syndrome “In the sleep laboratory, the 2 conditions can be distinguished on the basis of alpha intrusion, which was only present in the patients with FM, suggesting the potential utility of PSG [polysomnography] along with ACR [American College of Rheumatology] guidelines in making a definitive diagnosis of FM.” On the contrary, alpha wave intrusion is in fact non-specific as Scharf had previously stated in document B. Scharf in document A and Scharf et al. in document B indicate that both fibromyalgia (ARG type II) and chronic fatigue syndrome (ARG type I) are associated with alpha wave intrusion but Scharf et al. in document C indicate that alpha wave intrusion is associated with fibromyalgia and not with chronic fatigue syndrome.

ARG is a clinical diagnosis not dependent on polysomnography. The ACR (American College of Rheumatology) criteria for fibromyalgia are misleading and only address a subset of patients with ARG type II, namely those with extensive trigger point tenderness, which is discussed below. Neither polysomnography nor abnormally tender trigger points are necessary for the diagnosis of ARG. The primary utility of polysomnography is diagnosis of other disorders which may effect sleep such as sleep apnea and periodic limb movement disorder, although alpha wave intrusion is associated with both ARG type I and ARG type II, regardless of the contradictory claims of Scharf or Scharf et al. Relying on alpha wave intrusion seen on polysomnography combined with ACR criteria does not address fatigue, which is often the patient's main complaint. In document A Scharf advised the same treatment for patients with both what he termed fibromyalgia and chronic fatigue syndrome, but in document C Scharf et al. state that their treatment is ineffective for chronic fatigue syndrome (ARG type I). Although ARG type II may be treated with the relatively low doses of GHB used by Scharf, the patients awaken quickly, the treatment is inconvenient, and treatment with a combination of GABAnergic agents is superior to Scharf's method.

Fibromyalgia is currently diagnosed based on pressing on muscular spots known as trigger points outlined by The American College of Rheumatology (The American Academy of Rheumatology 1990 Criteria for the Classification for Fibromyalgia. Report of the Multicenter Criteria Committee. Arthritis and Rheumatism, Vol. 33, No. 2 Feb. 1990). After not requiring fulfillment of ACR criteria in document B, Scharf et al. mistakenly concluded that GHB is ineffective in the treatment of chronic fatigue syndrome, and they then excluded patients with chronic fatigue syndrome in document C and required that all patients in the said study meet ACR trigger point criteria for fibromyalgia. The quandary of both diagnosis and treatment of ARG is seen in the uncertainty of Scharf or Scharf et al. regarding ARG.

Scharf et al. in document B and C used two doses of a single GABAnergic agent (GHB) per sleep period. Scharf in document A disclosed previous treatment of two patients with narcolepsy and a concurrent diagnosis of ARG type II (fibromyalgia) and one patient with narcolepsy and a concurrent diagnosis of ARG type I (chronic fatigue syndrome) with GHB. No further GABAnergic agents were utilized. Scharf in document A treated 4 non-narcoleptic patients, all with a diagnosis of ARG type II (fibromyalgia) and one also with a diagnosis of chronic ARG type I (chronic fatigue syndrome), with NaGHB two to three times per night. No other GABAnergic agents were used.

Scharf et al. 1998 in document B administered 2.25 grams NaGHB at bedtime and again four hours later. They state “in some instances because of the short half life of the medication, subjects were unable to obtain more than 2-3 h of sleep per dose. In these cases the dosage was adjusted upwards slightly.” They do not disclose what is meant by “slightly upwards.” Scharf et al. also stated “A number of patients found themselves wide awake within two to three hours after the first dose and similarly after the second dose.” Some required an undisclosed increase in the dosage to prolong their sleep, others required the use of an additional undisclosed sedating agent. No other GABAnergic agents other than GHB were disclosed.

Scharf et al. 2003 in document C administered 3 grams of NaGHB at bedtime and a second dose about 4 h later. Whether some patients woke up prematurely “wide awake” was undisclosed. Six of 24 patients in the treatment arm withdrew due to side effects. No further GABAnergic or sedating agents were used. The highest dose of NaGHB used in either documents B or C was 6 grams in divided doses.

The sleep inducing effect of GHB in therapeutic doses is typically about 2-4 hours. A disadvantage of using GHB as monotherapy is that patients can only obtain about 2-4 hours sleep per dose or cannot sleep the whole night or day depending the patient's sleep cycle. Thus, the patient must inconveniently take more than one dose of GHB per sleep period, often requiring that an alarm clock awaken her to take the second dose. Furthermore, it is common that either the first or second dose induces a state of stupor but not sleep.

Physicians are ill-informed regarding appropriate use of GABAnergic agents. Physicians do not appreciate that GABAnergic agents may be used in combination and in large doses for the treatment of ARG.

### SUMMARY OF THE INVENTION

The present invention overcomes previous deficiencies by providing compositions that cannot be surreptitiously administered to an un-suspecting person. The compositions of the invention are readily discernible, even when mixed in a food and/or beverage. The compositions of the invention whether dilute or concentrated, contain one or more flavoring agent(s) or coloring agent(s).

The invention also provides solid state compositions of the sodium salt of gamma-hydroxyl butyric acid (NaGHB) and the sodium salt of 4-hydroxyl pentanoic acid.

The invention further provides methods to treat sleeping disorders, insomnia, drug abuse, alcohol and opiate withdrawal, a reduced level of growth hormone, anxiety, analgesia, acquired resistance to GABAnergic agents (ARG), effects in certain neurological disorders, such as Parkinson's Disease, depression, obesity, certain endocrine disturbances and tissue protection following hypoxia/anoxia such as in stroke or myocardial infarction, an increased level of intracranial pressure insomnia, anxiety, hyperactivity, attention deficit, attention deficit hyperactivity disorder, decreased concentration, depression, mania, hypomania, bipolar disorder, cylcothymia, sleepiness during waking hours, narcolepsy major, narcolepsy minor, restless legs syndrome, periodic limb movement disorder, subclinical restless legs syndrome, subclinical periodic limb movement disorder, obstructive sleep apnea, obstructive sleep apnea treated with continuous positive airway pressure therapy or oral device, or other conditions treatable with GHB. In particular, the invention provides methods of treating ARG.

Furthermore, the present invention relates generally to the field of pharmaceutical agents to be used in treating sleeping disorders, such as, e.g., narcolepsy, drug abuse, alcohol and opiate withdrawal, a reduced level of growth hormone, anxiety, analgesia, effects in certain neurological disorders such as Parkinson's Disease, depression, ARG, certain endocrine disturbances and tissue protection following hypoxia/anoxia such as in stroke or myocardial infarction, or for an increased level of intracranial pressure or the like.

Furthermore, the invention comprises treatment of ARG and the above identified diseases with one or more GABAnergic agents which are independently selected from Classes I, II, III, IV, V, and VI GABAnergic agents. The agents may be selected from the same Class of GABAnergic agents. The invention provides dilute and concentrated solutions of GHB; GBL; 1,4-butanediol; 4-HPA; and 4-HPA lactone for the treatment of ARG. When treating ARG and the above identified diseases, vitamins may be used as co-therapy.

### DETAILED DESCRIPTION OF PHARMACEUTICAL COMPOSITIONS

The invention further provides pharmaceutical compositions of gamma-hydroxyl butyric acid (GHB), pharmaceutically acceptable salts thereof; gamma-butyryl lactone; 1,4-butanediol; 4-hydroxyl pentanoic acid, pharmaceutically acceptable salts thereof; and 4-hydroxyl pentanoic acid lactone in an aqueous and/or an organic medium.

In an aspect, the organic medium contains ethyl alcohol. In certain preferred embodiments, an “aqueous medium” or an “organic medium” may be a solution, suspension, gel or emulsion of GHB, salts thereof; GBL; 1,4-butanediol; 4-HPA, salts thereof, and/or 4-HPA lactone with a solution being most preferred. Preferred gels are thixotropic gels. In an aspect, if the organic medium contains ethyl alcohol, the amount of ethyl alcohol used has minimal to no pharmacologic effect but rather is used to increase the solubility of additives.

GHB can also be used to treat insomnia, the symptoms associated with acquired resistance to GABAminergic agents, including fatigue (the feeling of tiredness), unrestful sleep, difficulty dealing with stress, arthralgias (joint pain), myalgias (muscle aching), tense muscles, difficulty concentrating (brain fog), impaired memory, depression, anxiety, insomnia, sleepiness, and tender trigger points, said treatment comprising administering a therapeutically effective amount of GHB to a patient in need of such treatment. In a preferred aspect the patient is a human. It is expected that GBL; 1,4-butanediol; 4-HPA; and 4-HPA lactone will have similar effects.

GHB; GBL; 1,4-butanediol; 4-HPA; and 4-HPA lactone are useful for treating ARG associated with restless legs syndrome, periodic limb movement disorder of sleep, interstitial cystitis, irritable bowel syndrome, rheumatoid arthritis, lupus erythematosus, and multiple sclerosis.

The invention also provides a method of preparing a dilute pharmaceutical composition of GHB; GBL; 1,4-butanediol; 4-HPA; or 4-HPA lactone for the prevention of sexual assault comprising admixing GHB; GBL; 1,4-butanediol; 4-HPA; or 4-HPA lactone and a coloring and/or flavoring agent in an aqueous and/or organic medium. In certain embodiments, the method of preparing the pharmaceutical composition further comprises admixing a preservative or buffering agent with the pharmaceutical composition.

In the invention, the GHB is formulated as a salt in an aqueous and/or organic solution. The preferred salts of gamma-hydroxyl butyric acid and 4-hydroxyl pentanoic acid are the sodium salts. In an aspect, the GHB is formulated as a salt in an aqueous solution that has a concentration of about 5 to 100 mg/ml of GHB salt per ml of solution. More preferably, the formulation has a concentration of about 10 to 50 mg/ml. Still more preferably, the formulation has a concentration of about 14 to 30 mg/ml. Yet more preferably, the formulation has a concentration of about 15 to 20 mg/ml.

An advantage of dilute solutions over concentrated solutions is as follows. Concentrated GHB solutions, such as solutions containing 500 mg/ml can easily be surreptitiously administered to an un-suspecting person because, volume wise, it takes very little solution to administer an intoxifying amount of GHB. For example, administering 1.5 g of GHB would require only 3 ml of 500 mg/ml solution. Adding this small volume of liquid to a standard 12 ounce bottle of beer or a six ounce glass of wine or cocktail will not noticeably affect the total volume of the beverage. Conversely, it is virtually impossible to surreptitiously administer an intoxifying amount of a dilute solution of GHB. To administer 1.5 g of GHB with a dilute solution (20 mg/ml) of GHB requires 75 ml of solution. Adding this much solution to a drink would noticeably increase the volume of the drink. Of course, an even more dilute solution will be even harder to surreptitiously add to an un-suspecting person's food of beverage.

Concentrated pharmaceutical solutions of GHB; GBL; 1,4-butanediol; 4-HPA; and 4-HPA lactone with a distinctive taste and/or color are useful for treating ARG and related conditions. Concentrated formulations decrease shipping and storage costs, while the flavoring and/or coloring discourages the surreptitious administration of the concentrated solution to an unsuspecting person.

The chemical stability of GHB is affected by pH. Accordingly, the method for preparing GHB, as described herein, varies with pH. Gamma-butyryl lactone (GBL the cyclized form of GHB) begins to form if the pH is about 6 or less. Compositions with a pH of greater than about 6.0 are preferred to produce chemically stable (i.e., those that don't cyclize) formulations of GHB. Thus, a preferred range to produce chemically stable GHB would be from about pH 6 to about pH 9. However, any pH or range of pH values where a clinically acceptable amount of GBL is produced is also contemplated as being preferred, and is encompassed by the present invention. The range of GBL could be regulatorily broadened with availability of sufficient toxicological data. To achieve the desired range, a buffering agent or some other agent may be added to maintain the desired pH. 4-HPA is expected to become cyclic at sufficiently acidic pH.

The pH of NaGHB at 240 mg per ml is approximately 9.5, at which neither pathogenic nor non-pathogenic organisms grow. As the concentration of NaGHB is increased, the pH is yet higher. Thus, highly concentrated formulations of NaGHB are inherently resistent to microbial growth and do not require preservative. Concentrated NaGHB is highly toxic regardless of pH and must be diluted prior to consumption. The pH of NaGHB at 500 mg/ml drops rapidly into the physiologic range upon dilution. Highly concentrated GBL and 1,4-butanediol are known to be resistant to microbial growth, require no special storage procedures, and likewise must be diluted prior to consumption, typically to 5% to 30% solutions but more preferably 5% to 20%.

The formulations of the invention which are used in preventing date rape or sexual assault contain at least one flavoring agent, a coloring agent or a combination thereof. While any flavoring agent may be used, the flavoring agent should be strong, i.e., not easily masked by the taste of soda, beer, or other alcoholic beverages. The flavor(s) of the invention include natural and artificial flavors. These flavorings may be chosen from synthetic flavor oils and flavoring aromatics, and/or oils, oleo resins and extracts derived from plants, leaves, flowers, fruits and so forth, and combinations thereof. The flavoring agent may be pleasant tasting, or it may be not so pleasing. Flavors useful in the invention include, for example, beets, garlic, fatty, oily, fishy, soy, menthol, mentholyptus, asparagus, onion, grapefruit, spinach, mustard, crab, shrimp, clam, pickles, olives, pepper, molasses, mayonnaise, butyric acid, mint, spearmint, pecan, walnut, cashew, pumpkin, bacon, steak, ham, lettuce, carrot, cauliflower, celery, broccoli, cucumber, green bean, lima bean, black bean, peas, squash, corn, potato, yam, cheese, cottage cheese, mushroom, chocolate, chocolate mint, peanut butter, caramel, strawberry, cherry, blackberry, grape, banana, peach, apricot, cranberry, blueberry, rasberry, lemon, orange, pear, raisin, cantaloupe, bread, coffee, prunes, yoghurt, cyclamic acid, or milk. Flavoring agents used may be menthol, menthone, carvone, vanillin, benzaldehyde, methyl butyrate, ethyl acetate, cinnamic aldehyde, ethyl butanoate, pentyl butanoate, monosodium glutamate, disodium 5′-inosinate, disodium 5′-guanylate, annato extract, B-Apo-8′-caretenal, dehydrated beet powder, canthaxanthin, caramel, B-Carotene, carrot oil, ferrous gluconate, grape skin extract, paprika, paprika oleoresin, riboflavin, saffron, titanium dioxide, turmeric, turmeric oleoresin, zingiberone, p-anisic acid, cinnamic acid, phenylacetic acid, d,l-bomeol, d-borneol, 1-borneol, carvacrol, chavicol, cinnamyl alcohol, linalool, menthol, nerolidol, nerol, d,l-.alpha.-terpineol, d-.alpha.-terpineol, 1-.alpha.-terpineol, thymol, acetaldehyde, anisaldehyde, cinnamaldehyde, benzaldehyde, citral, isovaleric aldehyde, piperonal, valeric aldehyde, vanillin, carvone, jasmone, menthone, piperitone, amyl acetate, bornyl acetate, benzyl benzoate, butyl cinnamate, cinnamyl anthranilate, geranyl acetate, linalyl acetate, menthyl acetate, menthyl isovalerate, eugenol, safrol, estragole, lemon oil, lime oil, neroli oil, orange oil, peppermint oil, spearmint oil, anise oil, cardamom oil, cinnamon oil, clove oil, coriander oil, eriodictyon fluidextract, eucalyptus oil, fennel oil, glycyrrhiza extract, lemongrass oil, nutmeg oil, and combinations thereof.

Still other flavors of the invention include tart, cinnamon oil, peppermint oil, clove oil, bay oil, thyme oil, cedar leaf oil, oil of nutmeg, oil of sage, and oil of bitter almonds. Also useful are artificial, natural or synthetic fruit flavors such as vanilla, chocolate, coffee, cocoa and citrus oil, including lemon, orange, grape, lime and grapefruit and fruit essences including apple, pear, peach, strawberry, raspberry, cherry, plum, pineapple, apricot and so forth. These flavorings can be used individually or in admixture. Commonly used flavors include mints such as peppermint, wintergreen, spearmint, birch, anise and such fruit flavors, as cherry, lemon-lime, orange, grape, artificial vanilla, cinnamon derivatives, and others, whether employed individually or in admixture. Flavorings such as aldehydes and esters including cinnamyl acetate, cinnamaldehyde, citral, diethylacetal, dihydrocarvyl acetate, eugenyl formate, p-methylanisole, and so forth may also be used. Generally, any flavoring or food additive, such as those described in Chemicals Used in Food Processing, publication 1274 by the National Academy of Sciences, pages 63-258, may be used. Further examples of aldehyde flavorings include, but are not limited to acetaldehyde (apple); benzaldehyde (cherry, almond); cinnamic aldehyde (cinnamon); citral, i.e., alpha citral (lemon, lime); neral, i.e. beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavors); butyraldehyde (butter, cheese); valeraldehyde (butter, cheese); citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 2,6-dimethyl-5-heptenal, i.e. melonal (melon); 2-6-dimethyloctanal (green fruit); and 2-dodecenal (citrus, mandarin); cherry; grape; mixtures thereof; and the like.

Preferred flavoring agents include menthol, menthone, vanillin, benzaldehyde, methyl butyrate, ethyl acetate, cinnamic aldehyde, or combinations thereof. In an aspect, the preferred flavor is minty and the preferred flavoring agents used to produce a minty taste are menthol, menthone, or combinations thereof. Menthol and menthone have not only a minty taste but also a taste associated with medication. Carvone, spearmint, and essential oil of spearmint may also be used to produce a minty taste. In a preferred aspect, the taste is tart and the preferred flavoring agents used to produce a tart taste are citric acid, tartaric acid, or combinations thereof.

If desired, high fructose corn syrup, and/or other sweeteners (such as sucrose, saccharin, aspartame, saccharin, sodium saccharin, calcium saccharin, sucralose, acesulfame-K, sorbitol, xylitol, steviosin, steviol, mannitol, erythritol, lactitol, xylose, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, and glycyrrhizin, cyclamate salts, the sodium, ammonium or calcium salt of 3,4-dihydro-6-methyl-1,2,3- -1-oxathiazine-4-one-2,2-dioxide, dipeptide based sweeteners, such as L-aspartic acid derived sweeteners, such as L-aspartyl-L-phenylalanine methyl ester (aspartame) and materials described in U.S. Pat. No. 3,492,131, L-alpha-aspartyl-N-(2,2,4,4-tetramethyl-3-thietanyl)-D-alaninamide hydrate, methyl esters of L-aspartyl-L-phenylglycerin and L-aspartyl-L-2,5,dihydrophenyl-glycine, L-aspartyl-2,5-dihydro-L-phenylal-1-anine, L-aspartyl-L-(1-cyclohexyen)-alanine, and mixtures thereof), may also be added.

The formulations of the invention optionally comprise a coloring agent that provides the GHB solution with a distinctive color that is identifiable, even after being added to a food and/or beverage. Examples of coloring agents include, for example, FD & C Blue No. 1, FD & C Blue No. 2, FD & C Green 1, FD & C Green No. 2, FD & C Green No. 3, FD & C Orange 1, FD & C Red No. 1, FD & C Red No. 2, FD & C Red No. 3, FD & C Red No. 4, FD & C Red No. 32, FD & C Red No. 40, FD & C Yellow No. 1, FD & C Yellow No. 3, FD & C Yellow No. 4, FD & C Yellow No. 5, FD & C Yellow No. 6. Colors that are readily observed by the un-aided eye are preferred.

The non-toxic pharmaceutically acceptable salts of the invention include, but are not limited to, salts of inorganic acids such as hydrochloric, sulfuric, phosphoric, diphosphoric, hydrobromic, and nitric or salts of organic acids such as formic, citric, malic, maleic, fumaric, tartaric, succinic; acetic, lactic, methanesulfonic, p-toluenesulfonic, 2-hydroxyethylsulfonic, salicylic and stearic. Similarly, pharmaceutically acceptable cations include, but are not limited to sodium, potassium, calcium, aluminum, lithium and ammonium. Preferred GHB and 4-HPA salts of the present invention include sodium, potassium, lithium, ammonium and calcium. The preferred salts are the sodium salts. Those of ordinary skill in the field of the invention will recognize a wide variety of non-toxic pharmaceutically acceptable addition salts.

Preservatives useful in the invention include sodium benzoate, methyl paraben, ethyl paraben, propyl paraben, butyl paraben, other parabens up to heptyl paraben, sodium methylparahydroxybenzoate, sodium ethylparahydroxybenzoate, sodium propylparahydroxybenzoate, sorbate, sorbic acid, benzoate, benzoic acid, sodium propionate, propionic acid, lactate, lactic acid, citric acid, malic acid, acetic acid, phosphoric acid, carbonic acid, hydrochloric acid, nisin, pimaricin (also known as natamycin), ethylenediamine tetra-acetic acid (EDTA), medium chain fatty-acids like octanoic acid, nonanoic acid, decoanoic acid, undecanoic acid, lauric acid, sodium metabisulfite, sodium sulfite, calcium bisulfite, sodium nitrite, trisodium phosphate, hydrogen peroxide, chlorine, chlorine dioxide, benzalkonium chloride, benzethonium chloride, benzyl alcohol, cetylpyridium chloride, ethylenediamine, phenylethyl alcohol, thimerosal, esters of gamma-hydroxybutric acid, methyl jasmonate, mint essential oils, trans-cinnamaldehyde, cinnamic acid, carvacrol, cymene, cyclamic acid, sodium cyclamate, etruscomycin, lactoperoxidase, lactoferrin, allyl isothiocyanate, diacetyl, reuterin (3-hydroxypropionaldehyde), pediocin, lactococcin G, lactacin F, plantaricin S. and combinations thereof. Preferred preservatives include sodium benzoate, methyl paraben, ethyl paraben, propyl paraben, and combinations thereof. Preferred preservatives above a pH of 5 include sodium benzoate, methyl paraben, ethyl paraben, propyl paraben, and combinations thereof. Preferred preservatives below a pH of about 5 include organic acids and salts thereof including sorbic acid, calcium sorbate, potassium sorbate, sodium sorbate, citric acid, formic acid, propionic acid, calcium propionate, potassium propionate, sodium propionate, benzoic acid, calcium benzoate, potassium benzoate, sodium benzoate, and lactic acid. Preferred preservatives below a pH of about 5 also include methyl paraben, ethyl paraben, propyl paraben, or combinations thereof.

Surfactants include non-ionic hydrophilic or ionic surfactants or lipophilic additives selected from among alkylglucosides, alkylmaltosides, alkylthioglucosides, lauryl macrogolglycerides, polyoxyethylene alkyl ethers, polyoxyethylene alkylphenols, polyethylene glycol fatty acids esters, polyethylene glycol glycerol fatty acid esters, polyoxyethylene sorbitan fatty acid esters, polyoxyethylene-polyoxypropylene block copolymers, polyglycerol fatty acid esters, polyoxyethylene glycerides, polyoxyethylene sterols, derivatives, and analogues thereof, polyoxyethylene vegetable oils, polyoxyethylene hydrogenated vegetable oils, reaction mixtures of polyols and at least one member of the group consisting of fatty acids, glycerides, vegetable oils, hydrogenated vegetable oils, and sterols, tocopherol polyethylene glycol succinates, sugar esters, sugar ethers; sucroglycerides, and mixtures thereof.

Ionic surfactants may be selected from among alkyl ammonium salts; bile acids and salts, analogues, and derivatives thereof; fatty acid derivatives of amino acids, carnitines, oligopeptides, and polypeptides; glyceride derivatives of amino acids, oligopeptides, and polypeptides; acyl lactylates; mono-diacetylated tartaric acid esters of mono-diglycerides; succinylated monoglycerides; citric acid esters of mono-diglycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; and mixtures thereof.

In a preferred embodiment, the invention provides a dilute GHB solution comprising the GHB in a concentration of about 20 mg/ml, a buffer or other additive to maintain the pH between about 6 and 9 (more preferably, between a pH of about 6.5 and 8.5), at least one preservative, at least one flavoring agent, and at least one coloring agent. If desired, surfactants may also be added to the formulation. In another aspect, the pH of the solution is 7-8.2.

The invention provides a GHB solution comprising the GHB in a concentration of greater than 100 mg/ml, a buffer or other additive to maintain the pH between about 6 and 11 (more preferably, between a pH of about 6.5 and 8.5), at least one preservative, at least one flavoring agent, and at least one coloring agent. If desired, surfactants may also be added to the formulation. In another aspect, the pH of the solution is 7-8.2.

In an aspect, the invention provides a dilute GHB solution comprising the GHB in a concentration of about 20 mg/ml, a buffer or other additive to maintain the pH between about 6 and 9 (more preferably, between a pH of about 6.5 and 8.5), at least one preservative, and at least one flavoring agent. If desired, surfactants may also be added to the formulation. In another aspect, the pH is about 7 to 8.2.

The invention provides a GHB solution comprising the GHB in a concentration of greater than 100 mg/ml, a buffer or other additive to maintain the pH between about 6 and 11 (more preferably, between a pH of about 6.5 and 8.5), at least one preservative, and at least one flavoring agent. If desired, surfactants may also be added to the formulation. In another aspect, the pH is about 7 to 8.2.

In an aspect, the invention provides a dilute GHB solution comprising the GHB in a concentration of about 20 mg/ml, a buffer or other additive to maintain the pH between about 6 and 9 (more preferably, between a pH of about 6.5 and 8.5), at least one preservative, and at least one coloring agent. If desired, surfactants may also be added to the formulation. In another aspect, the pH is about 7 to 8.2.

The invention provides a GHB solution comprising the GHB in a concentration of greater than 100 mg/ml, a buffer or other additive to maintain the pH between about 6 and 11 (more preferably, between a pH of about 6.5 and 8.5), at least one preservative, and at least one coloring agent. If desired, surfactants may also be added to the formulation. In another aspect, the pH is about 7 to 8.2.

In an aspect, the invention provides a dilute GHB solution comprising the GHB in a concentration of about 20 mg/ml, a buffer or other additive to maintain the pH between about 6 and 9 (more preferably, between a pH of about 6.5 and 8.5), and at least one preservative. If desired, surfactants may also be added to the formulation. In another aspect, the pH is about 7 to 8.2.

The invention provides a GHB solution comprising the GHB in a concentration of greater than 100 mg/ml, a buffer or other additive to maintain the pH between about 6 and 11 (more preferably, between a pH of about 6.5 and 8.5), and optionally at least one preservative. If desired, surfactants may also be added to the formulation. In another aspect, the pH is about 7 to 8.2.

In an aspect, the invention provides a dilute GHB solution comprising the GHB in a concentration of about 20 mg/ml, and at least one preservative. If desired, surfactants may also be added to the formulation. In another aspect, the pH is about 7 to 8.2.

In an aspect, the invention provides a GHB solution comprising the GHB in a concentration of greater than 100 mg/ml, and optionally at least one preservative. If desired, surfactants may also be added to the formulation. In another aspect, the pH is about 7 to 8.2.

In another aspect, the invention provides a GHB solution at a pH of about 2.2 to 9.0; furthermore the solution contains GHB in a concentration from about 5 to about 100 mg/ml. More preferably, the pH is about 6.0 to about 8.2. A buffer or pH adjusting agent may be added to obtain the desired pH. A flavoring agent may be optionally added. A coloring agent may optionally be added.

In another aspect, the invention provides a dilute GHB solution comprising the GHB in a concentration of about 20 mg/ml contained in a sterile bag or container which may be opened prior to consumption.

If needed or desired, when treating a condition that responds to the administration of GHB, multiple doses of the dilute solution may be administered.

In certain embodiments of the invention, the solution comprises a dilute solution of GHB, a pH adjusting agent, and a preservative. Phosphoric acid, hydrogen chloride, sodium hydroxide, and potassium hydroxide may be used as pH adjusting agents. The pH adjusting agent may also serve as a preservative, such as citric acid, sorbic acid, benzoic acid, propionic acid, lactic acid, citric acid, malic acid, acetic acid, phosphoric acid, carbonic acid, and cinnamic acid, or combinations thereof.

In certain embodiments of the invention, the solution comprises GHB, a pH adjusting agent, a preservative, and a buffer. The pH adjusting agent, preservative and buffering agent may be a combination of an acid and a salt thereof, or a combination of an acid and a salt of another acid. Such agents include citric acid, sorbic acid, benzoic acid, propionic acid, lactic acid, citric acid, malic acid, acetic acid, phosphoric acid, carbonic acid, cinnamic acid, and salts thereof. Pharmaceutically acceptable cations of salts include, but are not limited to sodium, potassium, calcium, aluminum, lithium and ammonium.

In certain embodiments of the invention, the solution comprises GHB and/or GBL and/or 1,4-butanediol, a pH adjusting agent, a preservative, and a buffer. In an aspect, the invention is a combination of GHB and GBL. The pH may be from about 2.2-9.0. The pH adjusting agent, preservative and buffering agent may be a combination of an acid and a salt thereof, or a combination of an acid and a salt of another acid. Such agents include citric acid, sorbic acid, benzoic acid, propionic acid, lactic acid, citric acid, malic acid, acetic acid, phosphoric acid, carbonic acid, cinnamic acid, and salts thereof. Pharmaceutically acceptable cations of salts include, but are not limited to sodium, potassium, calcium, aluminum, lithium and ammonium.

The invention also provides a method of preparing a dilute pharmaceutical composition for the treatment of a condition responsive to the drugs, comprising admixing GHB; GBL; and/or 1,4-butanediol and water or an organic medium. In a preferred aspect, the water or organic medium also contains a pH adjusting and/or buffering agent. In certain embodiments, the method of preparing the pharmaceutical composition further comprises admixing a preservative with the pharmaceutical composition. Other components, such as flavoring agents, salts, and the like, may be added to the composition.

In certain other embodiments, the method of preparing the pharmaceutical composition comprises admixing GHB, GB, and/or 1,4-butanediol, a pH adjusting or buffering agent, and an aqueous and/or organic medium soon before administration to a patient suspected of having a condition responsive to the medications.

The invention also provides a method of treating any therapeutic category of disorder responsive to GHB; GBL; and/or 1,4-butanediol, comprising administering to a patient suspected of having such a condition a therapeutic amount of a pharmaceutical composition comprising GHB and/or GBL and/or 1,4-butanediol in a dilute or concentrated aqueous medium. In an aspect, the pharmaceutical composition comprises about 4-48 mmol of GHB; GBL; or 1,4-butanediol.

In one aspect of the invention, the GBL or 1,4-butanediol is formulated in an aqueous solution that has a concentration of about 3 to 68 mg/ml of GBL per ml of solution, or correspondingly about 3 to 71 mg/ml 1,4-butanediol. More preferably, the formulation has a concentration of about 7 to 34 mg/ml of GBL, or correspondingly about 7 to 36 mg/ml 1,4-butanediol. Still more preferably, the formulation has a concentration of about 10 to 20 mg/ml GBL or correspondingly 10 to 21 mg/ml 1,4-butanediol. Yet more preferably, the formulation has a concentration of about 10 to 14 mg/ml GBL or correspondingly 11 to 14 mg/ml 1,4-butanediol.

In another aspect, more concentrated solutions containing up to 800 mg/ml GBL or up to 800 mg/ml 1,4-butanediol are preferred. However, the patient should be instructed to dilute the solution to avoid GI tract toxicity. Solutions of even higher concentration may be dispensed to the patient, but would require the patient to dilute the solution.

In one aspect of the invention, the pH of the solutions may be between 2.2 and 9.0.

In a preferred embodiment, the invention also provides a dilute GBL solution comprising the GBL in a concentration of about 14 mg/ml, a buffer or other additive to maintain the pH between about 5 and 9 (more preferably, between a pH of about 6 and 8.5), at least one preservative, at least one flavoring agent, and at least one coloring agent. If desired, surfactants may also be added to the formulation. In another aspect, the pH of the solution is 6-8.2.

In an aspect, the invention also provides a dilute GBL solution comprising the GBL in a concentration of about 14 mg/ml, a buffer or other additive to maintain the pH between about 5 and 9 (more preferably, between a pH of about 6 and 8.5), at least one preservative, and at least one flavoring agent. If desired, surfactants may also be added to the formulation. In another aspect, the pH of the solution is 6-8.2.

In an aspect, the invention also provides a dilute GBL solution comprising the GBL in a concentration of about 14 mg/ml, a buffer or other additive to maintain the pH between about 5 and 9 (more preferably, between a pH of about 6 and 8.5), at least one preservative, and at least one coloring agent. If desired, surfactants may also be added to the formulation. In another aspect, the pH of the solution is 6-8.2.

In an aspect, the invention also provides a dilute GBL solution comprising the GBL in a concentration of about 14 mg/ml, a buffer or other additive to maintain the pH between about 5 and 9 (more preferably, between a pH of about 6 and 8.5), and at least one preservative. If desired, surfactants may also be added to the formulation. In another aspect, the pH of the solution is 6-8.2.

In an aspect, the invention provides a concentrated GBL solution comprising the GBL in a concentration exceeding 68 mg/ml, a buffer or other additive to maintain the pH between about 5 and 11 (more preferably, between a pH of about 6 and 8.5), at least one preservative, at least one flavoring agent, and at least one coloring agent. If desired, surfactants may also be added to the formulation. In another aspect, the pH of the solution is 6-8.2.

In an aspect, the invention also provides a concentrated GBL solution comprising the GBL in a concentration exceeding 68 mg/ml, a buffer or other additive to maintain the pH between about 5 and 11 (more preferably, between a pH of about 6 and 8.5), at least one preservative, and at least one flavoring agent. If desired, surfactants may also be added to the formulation. In another aspect, the pH of the solution is 6-8.2.

In an aspect, the invention also provides a concentrated GBL solution comprising the GBL in a concentration exceeding 68 mg/ml, a buffer or other additive to maintain the pH between about 5 and 11 (more preferably, between a pH of about 6 and 8.5), at least one preservative, and at least one coloring agent. If desired, surfactants may also be added to the formulation. In another aspect, the pH of the solution is 6-8.2.

In an aspect, the invention also provides a concentrated GBL solution comprising the GBL in a concentration exceeding 68 mg/ml, a buffer or other additive to maintain the pH between about 5 and 11 (more preferably, between a pH of about 6 and 8.5), and at least one preservative. If desired, surfactants may also be added to the formulation. In another aspect, the pH of the solution is 6-8.2.

In a preferred aspect, the invention provides a dilute 1,4-butanediol solution comprising the 1,4-butanediol in a concentration of about 14 mg/ml, a buffer or other additive to maintain the pH between about 5 and 9 (more preferably, between a pH of about 6 and 8.5), at least one preservative, at least one flavoring agent, and at least one coloring agent. If desired, surfactants may also be added to the formulation. In another aspect, the pH is about 6 to 8.2.

In an aspect, the invention provides a dilute 1,4-butanediol solution comprising the 1,4-butanediol in a concentration of about 14 mg/ml, a buffer or other additive to maintain the pH between about 5 and 9 (more preferably, between a pH of about 6 and 8.5), at least one preservative, and at least one flavoring agent. If desired, surfactants may also be added to the formulation. In another aspect, the pH is about 6 to 8.2.

In an aspect, the invention provides a dilute 1,4-butanediol solution comprising the 1,4-butanediol in a concentration of about 14 mg/ml, a buffer or other additive to maintain the pH between about 5 and 9 (more preferably, between a pH of about 6 and 8.5), at least one preservative, and at least one coloring agent. If desired, surfactants may also be added to the formulation. In another aspect, the pH is about 6 to 8.2.

In an aspect, the invention provides a dilute 1,4-butanediol solution comprising the 1,4-butanediol in a concentration of about 14 mg/ml, a buffer or other additive to maintain the pH between about 5 and 9 (more preferably, between a pH of about 6 and 8.5), and at least one preservative. If desired, surfactants may also be added to the formulation. In another aspect, the pH is about 6 to 8.2.

The invention provides a concentrated 1,4-butanediol solution comprising the 1,4-butanediol in a concentration exceeding 71 mg/ml, a buffer or other additive to maintain the pH between about 5 and 11 (more preferably, between a pH of about 6 and 8.5), at least one preservative, at least one flavoring agent, and at least one coloring agent. If desired, surfactants may also be added to the formulation. In another aspect, the pH of the solution is 6-8.2.

In an aspect, the invention provides a concentrated 1,4-butanediol solution comprising the 1,4-butanediol in a concentration exceeding 71 mg/ml, a buffer or other additive to maintain the pH between about 5 and 11 (more preferably, between a pH of about 6 and 8.5), at least one preservative, and at least one flavoring agent. If desired, surfactants may also be added to the formulation. In another aspect, the pH of the solution is 6-8.2.

In an aspect, the invention provides a concentrated 1,4-butanediol solution comprising the 1,4-butanediol in a concentration exceeding 71 mg/ml, a buffer or other additive to maintain the pH between about 5 and 11 (more preferably, between a pH of about 6 and 8.5), at least one preservative, and at least one coloring agent. If desired, surfactants may also be added to the formulation. In another aspect, the pH of the solution is 6-8.2.

In an aspect, the invention provides a concentrated 1,4-butanediol solution comprising the 1,4-butanediol in a concentration exceeding 71 mg/ml, a buffer or other additive to maintain the pH between about 5 and 11 (more preferably, between a pH of about 6 and 8.5), and at least one preservative. If desired, surfactants may also be added to the formulation. In another aspect, the pH of the solution is 6-8.2.

If needed or desired, when treating a condition that responds to the administration of GBL or 1,4-butanediol, multiple doses of a dilute or concentrated solution may be administered.

In certain embodiments of the invention, the solution comprises GHB; GBL; and/or 1,4-butanediol, a pH adjusting and a preservative. Phosphoric acid, hydrogen chloride, sodium hydroxide, and potassium hydroxide may be used as pH adjusting agents. The pH adjusting agent may also serve as a preservative, such as citric acid, sorbic acid, benzoic acid, propionic acid, lactic acid, citric acid, malic acid, acetic acid, phosphoric acid, carbonic acid, and cinnamic acid, or combinations thereof.

In certain embodiments of the invention, the solution comprises GHB; GBL; and/or 1,4-butanediol, a pH adjusting agent, a preservative, and a buffer. The pH adjusting agent, preservative and buffering agent may be a combination of an acid and a salt thereof, or a combination of an acid and a salt of another acid. Such agents include citric acid, sorbic acid, benzoic acid, propionic acid, lactic acid, citric acid, malic acid, acetic acid, phosphoric acid, carbonic acid, cinnamic acid, and salts thereof.

In a preferred aspect, the invention provides a dilute GBL solution comprising the GBL in a concentration of about 14 mg/ml, a buffer or other additive to maintain the pH between about 2.2 and 7, at least one preservative, at least one flavoring agent, and at least one coloring agent. More preferably, the pH is between about 2.2 and 5. Yet more preferably, the pH is between about 3 and 3.5. If desired, surfactants may also be added to the formulation.

In an aspect, the invention provides a dilute GBL solution comprising the GBL in a concentration of about 14 mg/ml, a buffer or other additive to maintain the pH between about 2.2 and 7, at least one preservative, and at least one flavoring agent. More preferably, the pH is between about 2.2 and 5. Yet more preferably, the pH is between about 3 and 3.5. If desired, surfactants may also be added to the formulation.

In an aspect, the invention provides a dilute GBL solution comprising the GBL in a concentration of about 14 mg/ml, a buffer or other additive to maintain the pH between about 2.2 and 7, at least one preservative, and at least one coloring agent. More preferably, the pH is between about 2.2 and 5. Yet more preferably, the pH is between about 3 and 3.5. If desired, surfactants may also be added to the formulation.

In an aspect, the invention provides a dilute GBL solution comprising the GBL in a concentration of about 14 mg/ml, a buffer or other additive to maintain the pH between about 2.2 and 7, and at least one preservative. More preferably, the pH is between about 2.2 and 5. Yet more preferably, the pH is between about 3 and 3.5. If desired, surfactants may also be added to the formulation.

In an aspect, the invention provides a concentrated GBL solution comprising the GBL in a concentration exceeding 68 mg/ml, a buffer or other additive to maintain the pH between about 2.2 and 7, at least one preservative, at least one flavoring agent, and at least one coloring agent. More preferably, the pH is between about 2.2 and 5. Yet more preferably, the pH is between about 3 and 3.5. If desired, surfactants may also be added to the formulation.

In an aspect, the invention provides a concentrated GBL solution comprising the GBL in a concentration exceeding 68 mg/ml, a buffer or other additive to maintain the pH between about 2.2 and 7, at least one preservative, and at least one flavoring agent. More preferably, the pH is between about 2.2 and 5. Yet more preferably, the pH is between about 3 and 3.5. If desired, surfactants may also be added to the formulation.

In an aspect, the invention provides a concentrated GBL solution comprising the GBL in a concentration exceeding 68 mg/ml, a buffer or other additive to maintain the pH between about 2.2 and 7, at least one preservative, and at least one coloring agent. More preferably, the pH is between about 2.2 and 5. Yet more preferably, the pH is between about 3 and 3.5. If desired, surfactants may also be added to the formulation.

In an aspect, the invention provides a concentrated GBL solution comprising the GBL in a concentration exceeding 68 mg/ml, a buffer or other additive to maintain the pH between about 2.2 and 7, and at least one preservative. More preferably, the pH is between about 2.2 and 5. Yet more preferably, the pH is between about 3 and 3.5. If desired, surfactants may also be added to the formulation.

In another embodiment, the invention provides a dilute 1,4-butanediol solution comprising the 1,4-butanediol in a concentration of about 14 mg/ml, a buffer or other additive to maintain the pH between about 2.2 and 7, at least one preservative, at least one flavoring agent, and at least one coloring agent. More preferably, the pH is between about 2.2 and 5. Yet more preferably, the pH is between about 3 and 3.5. If desired, surfactants may also be added to the formulation.

In another embodiment, the invention provides a dilute 1,4-butanediol solution comprising the 1,4-butanediol in a concentration of about 14 mg/ml, a buffer or other additive to maintain the pH between about 2.2 and 7, at least one preservative, and at least one flavoring agent. More preferably, the pH is between about 2.2 and 5. Yet more preferably, the pH is between about 3 and 3.5. If desired, surfactants may also be added to the formulation.

In another embodiment, the invention provides a dilute 1,4-butanediol solution comprising the 1,4-butanediol in a concentration of about 14 mg/ml, a buffer or other additive to maintain the pH between about 2.2 and 7, at least one preservative, and at least one coloring agent. More preferably, the pH is between about 2.2 and 5. Yet more preferably, the pH is between about 3 and 3.5. If desired, surfactants may also be added to the formulation.

In another embodiment, the invention provides a dilute 1,4-butanediol solution comprising the 1,4-butanediol in a concentration of about 14 mg/ml, a buffer or other additive to maintain the pH between about 2.2 and 7, and at least one preservative. More preferably, the pH is between about 2.2 and 5. Yet more preferably, the pH is between about 3 and 3.5. If desired, surfactants may also be added to the formulation.

In an aspect, the invention provides a concentrated 1,4-butanediol solution comprising the 1,4-butanediol in a concentration exceeding 71 mg/ml, a buffer or other additive to maintain the pH between about 2.2 and 7, at least one preservative, at least one flavoring agent, and at least one coloring agent. More preferably, the pH is between about 2.2 and 5. Yet more preferably, the pH is between about 3 and 3.5. If desired, surfactants may also be added to the formulation.

In an aspect, the invention provides a concentrated 1,4-butanediol solution comprising the 1,4-butanediol in a concentration exceeding 71 mg/ml, a buffer or other additive to maintain the pH between about 2.2 and 7, at least one preservative, and at least one flavoring agent. More preferably, the pH is between about 2.2 and 5. Yet more preferably, the pH is between about 3 and 3.5. If desired, surfactants may also be added to the formulation.

In an aspect, the invention provides a concentrated 1,4-butanediol solution comprising the 1,4-butanediol in a concentration exceeding 71 mg/ml, a buffer or other additive to maintain the pH between about 2.2 and 7, at least one preservative, and at least one coloring agent. More preferably, the pH is between about 2.2 and 5. Yet more preferably, the pH is between about 3 and 3.5. If desired, surfactants may also be added to the formulation.

In an aspect, the invention provides a concentrated 1,4-butanediol solution comprising the 1,4-butanediol in a concentration exceeding 71 mg/ml, a buffer or other additive to maintain the pH between about 2.2 and 7, and at least one preservative. More preferably, the pH is between about 2.2 and 5. Yet more preferably, the pH is between about 3 and 3.5. If desired, surfactants may also be added to the formulation.

In another aspect, the invention provides a concentrated GBL solution at a pH of about 2.2 to 11. More preferably, the pH is about 3.0 to 8.0. The solution comprises GBL in a concentration exceeding 68 mg/ml. A buffer or pH adjusting agent may be added to obtain the desired pH. A flavoring agent may be optionally added. A coloring agent may optionally be added.

In another aspect, the invention provides a concentrated 1,4-butanediol solution at a pH of about 2.2 to 11. More preferably, the pH is about 3.0 to 8.0. The solution comprises 1,4-butanediol in a concentration exceeding 71 mg/ml. A buffer or pH adjusting agent may be added to obtain the desired pH. A flavoring agent may be optionally added. A coloring agent may optionally be added.

In another aspect, the invention provides a dilute GBL solution at a pH of about 2.2 to 9.0. More preferably, the pH is about 3.0 to 8.0. The solution comprises GBL in a concentration of 3 to 68 mg/ml. A buffer or pH adjusting agent may be added to obtain the desired pH. A flavoring agent may be optionally added. A coloring agent may optionally be added.

In another aspect, the invention provides a dilute 1,4-butanediol solution at a pH of about 2.2 to 9.0. More preferably, the pH is about 3.0 to 8.0. The solution comprises 1,4-butanediol in a concentration of 3 to 71 mg/ml. A buffer or pH adjusting agent may be added to obtain the desired pH. A flavoring agent may be optionally added. A coloring agent may optionally be added.

In certain other embodiments, the method of preparing the pharmaceutical composition comprises admixing the Class II GABAnergic agents C₅-C₁₀ alkanoic carboxylic acids; pharmaceutically acceptable salts thereof; the corresponding gamma lactones; 1,4 C₅-C₁₀ alkanoic diols; or combinations thereof, a pH adjusting or buffering agent, and an aqueous or organic medium soon before administration to a patient suspected of having a condition responsive to the medications.

In certain embodiments of the invention, the solution comprises one or more agents selected from C₅-C₁₀ alkanoic carboxylic acids; pharmaceutically acceptable salts thereof; the corresponding gamma lactones; and 1,4 C₅-C₁₀ alkanoic diols; and a pH adjusting agent, a preservative, and a buffer. The preferred agents are 4-hydroxyl pentanoic acid, salts thereof; 4-hydroxyl pentanoic acid lactone; 1,4-pentanediol, or combinations thereof. The pH adjusting agent, preservative and buffering agent may be a combination of an acid and a salt thereof, or a combination of an acid and a salt of another acid. Such agents include citric acid, sorbic acid, benzoic acid, propionic acid, lactic acid, citric acid, malic acid, acetic acid, phosphoric acid, carbonic acid, cinnamic acid, and salts thereof.

In an aspect, the invention comprises one or more C₅-C₁₀ alkanoic carboxylic acids, salts thereof; the corresponding gamma lactones; or 1,4 C₅-C₁₀ alkanoic diols in an aqueous or organic solution. The preferred agents are 4-hydroxyl pentanoic acid, pharmaceutically acceptable salts thereof, 4-hydroxyl pentanoic acid lactone, or combinations thereof. The preferred salts are the sodium salts. In an aspect, the pH of the solution is 2.2-11. In an aspect, the molarity of the agents is 0.02 to 1.2 molar. In another aspect, the molarity is greater than 1.2 molar.

If needed or desired, when treating a condition that responds to the administration of 4-hydroxyl pentanoic acid, pharmaceutically acceptable salts thereof, or 4-hydroxyl pentanoic acid lactone, multiple doses of a dilute or concentrated solution may be administered.

The invention also provides a method of treating any therapeutic category of disorder responsive to C₅-C₁₀ alkanoic carboxylic acids; pharmaceutically acceptable salts thereof; the corresponding gamma lactones; and 1,4 C₅-C₁₀ alkanoic diols comprising administering to a patient suspected of having such a condition a therapeutic amount of a pharmaceutical composition comprising C₅-C₁₀ alkanoic carboxylic acids; pharmaceutically acceptable salts thereof; the corresponding gamma lactones; or 1,4 C₅-C₁₀ alkanoic diols agents in a dilute or concentrated aqueous and/or organic medium. In an aspect, the pharmaceutical composition comprises about 3-110 mmol of 4-hydroxyl pentanoic acid, salts thereof, or 4-hydroxyl pentanoic acid lactone.

In one aspect of the invention, the 4-hydroxyl pentanoic acid or salts thereof is formulated in a dilute aqueous or organic solution that has a concentration of about 3 to 170 mg/ml of the sodium salt of 4-hydroxyl pentanoic acid per ml of solution, or correspondingly about 3 to 120 mg/ml 4-hydroxyl pentanoic acid lactone. More preferably, the formulation has a concentration of about 9 to 70 mg/ml of the sodium salt of 4-hydroxyl pentanoic acid, or correspondingly about 6 to 50 mg/ml 4-hydroxyl pentanoic acid lactone. Still more preferably, the formulation has a concentration of about 12 to 40 mg/ml of the sodium salt of 4-hydroxyl pentanoic acid or correspondingly 8 to 30 mg/ml 4-hydroxyl pentanoic acid lactone. Yet more preferably, the formulation has a concentration of about 14 to 27 mg/ml of the sodium salt of 4-hydroxyl pentanoic acid or correspondingly 10 to 19 mg/ml 4-hydroxyl pentanoic acid lactone.

In a preferred aspect, the invention comprises concentrated solutions containing 170 up to 800 mg/ml of the sodium salt of 4-hydroxyl pentanoic acid or 120 up to 800 mg/ml 4-hydroxyl pentanoic acid lactone. However, the patient should be instructed to dilute the solution to avoid GI tract toxicity. Solutions of even higher concentration may be dispensed to the patient, but would require the patient to dilute the solution. Highly concentrated formulations of 4-hydroxyl pentanoic acid, salts thereof, 4-hydroxyl pentanoic acid lactone, and 1,4 pentane diol are expected to be resistant to microbial growth and require no special storage procedures.

In one aspect of the invention, the pH of the solutions may be between about 2.2 and 11, with the preferred pH between about 3 and 8. No large scale medicinal studies at any pH have been done to date regarding Class II GABAnergic agents. 4-hydroxyl pentanoic and salts thereof are expected to become cyclic and transform into 4-hydroxyl pentanoic acid lactone at sufficiently acidic pH.

In a preferred embodiment, the invention provides a dilute solution of the sodium salt of 4-hydroxyl pentanoic acid comprising the sodium salt of 4-hydroxyl pentanoic acid in a concentration of about 20 mg/ml, a buffer or other additive to maintain the pH between about 5 and 9 (more preferably, between a pH of about 6 and 8.5), at least one preservative, at least one flavoring agent, and at least one coloring agent. The preferred flavoring agents include menthol, menthone, and carvone. If desired, surfactants may also be added to the formulation. In another aspect, the pH is about 6 to 8.2. 4-hydroxyl pentanoic acid and salts thereof are expected to become cyclic at sufficiently acidic pH.

In an aspect, the invention provides a dilute solution of the sodium salt of 4-hydroxyl pentanoic acid comprising the sodium salt of 4-hydroxyl pentanoic acid in a concentration of about 20 mg/ml, and optionally a buffer or other additive to maintain the pH between about 5 and 9 (more preferably, between a pH of about 6 and 8.5). One or more preservatives, flavoring agents, coloring agents, or combinations thereof may be added. If desired, surfactants may also be added to the formulation.

In a preferred embodiment, the invention provides a concentrated solution of the sodium salt of 4-hydroxyl pentanoic acid comprising the sodium salt of 4-hydroxyl pentanoic in a concentration exceeding 170 mg/ml, a buffer or other additive to maintain the pH between about 5 and 11 (more preferably, between a pH of about 6 and 8.5), at least one preservative, at least one flavoring agent, and at least one coloring agent. The preferred flavoring agents include menthol, menthone, and carvone. If desired, surfactants may also be added to the formulation. In another aspect, the pH is about 6 to 8.2.

In an aspect, the invention provides a concentrated solution of the sodium salt of 4-hydroxyl pentanoic acid comprising the sodium salt of 4-hydroxyl pentanoic in a concentration exceeding 170 mg/ml, and optionally a buffer or other additive to maintain the pH between about 5 and 11 (more preferably, between a pH of about 6 and 8.5). One or more preservatives, flavoring agents, coloring agents, or combinations thereof may be added. If desired, surfactants may also be added to the formulation. In another aspect, the pH of the solution is 6-8.2.

In a preferred embodiment, the invention also provides a dilute 4-hydroxyl pentanoic acid lactone solution comprising the 4-hydroxyl pentanoic acid lactone in a concentration of about 14 mg/ml, a buffer or other additive to maintain the pH between about 5 and 9 (more preferably, between a pH of about 6 and 8.5), at least one preservative, at least one flavoring agent, and at least one coloring agent. The preferred flavoring agents include menthol, menthone, and carvone. If desired, surfactants may also be added to the formulation. In another aspect, the pH of the solution is 6-8.2.

In an aspect, the invention also provides a dilute 4-hydroxyl pentanoic acid lactone solution comprising the 4-hydroxyl pentanoic acid lactone in a concentration of about 14 mg/ml, and optionally a buffer or other additive to maintain the pH between about 5 and 9 (more preferably, between a pH of about 6 and 8.5). One or more preservatives, flavoring agents, coloring agents, or combinations thereof may be added. If desired, surfactants may also be added to the formulation. In another aspect, the pH of the solution is 6-8.2.

In a preferred embodiment, the invention also provides a concentrated 4-hydroxyl pentanoic acid lactone solution comprising the 4-hydroxyl pentanoic acid lactone in a concentration exceeding 120 mg/ml, a buffer or other additive to maintain the pH between about 5 and 11 (more preferably, between a pH of about 6 and 8.5), at least one preservative, at least one flavoring agent, and at least one coloring agent. Preferred flavoring agents include menthol, menthone, and carvone. If desired, surfactants may also be added to the formulation. In another aspect, the pH of the solution is 6-8.2.

In an aspect, the invention also provides a concentrated 4-hydroxyl pentanoic acid lactone solution comprising the 4-hydroxyl pentanoic acid lactone in a concentration exceeding 120 mg/ml, and optionally a buffer or other additive to maintain the pH between about 5 and 11 (more preferably, between a pH of about 6 and 8.5). One or more preservatives, flavoring agents, coloring agents, or combinations thereof may be added. If desired, surfactants may also be added to the formulation. In another aspect, the pH of the solution is 6-8.2.

In a preferred aspect, the invention provides a dilute solution of the sodium salt of 4-hydroxyl pentanoic acid comprising the sodium salt of 4-hydroxyl pentanoic acid in a concentration of about 20 mg/ml, a buffer or other additive to maintain the pH between about 2.2 and 7, at least one preservative, at least one flavoring agent, and at least one coloring agent. Preferred flavoring agents include menthol, menthone, and carvone. More preferably, the pH is between about 2.2 and 5. Yet more preferably, the pH is between about 3 and 3.5. If desired, surfactants may also be added to the formulation.

In an aspect, the invention provides a dilute solution of the sodium salt of 4-hydroxyl pentanoic acid comprising the sodium salt of 4-hydroxyl pentanoic acid in a concentration of about 20 mg/ml, and optionally a buffer or other additive to maintain the pH between about 2.2 and 7. One or more preservatives, flavoring agents, coloring agents, or combinations thereof may be added. Yet more preferably, the pH is between about 3 and 3.5. If desired, surfactants may also be added to the formulation.

In a preferred aspect, the invention provides a concentrated solution of the sodium salt of 4-hydroxyl pentanoic acid comprising the sodium salt of 4-hydroxyl pentanoic acid in a concentration exceeding 170 mg/ml, a buffer or other additive to maintain the pH between about 2.2 and 7, at least one preservative, at least one flavoring agent, and at least one coloring agent. The preferred flavoring agents include menthol, menthone, and carvone. More preferably, the pH is between about 2.2 and 5. Yet more preferably, the pH is between about 3 and 3.5. If desired, surfactants may also be added to the formulation.

In an aspect, the invention provides a concentrated solution of the sodium salt of 4-hydroxyl pentanoic acid comprising the sodium salt of 4-hydroxyl pentanoic acid in a concentration exceeding 170 mg/ml, and optionally a buffer or other additive to maintain the pH between about 2.2 and 7. One or more preservatives, flavoring agents, coloring agents, or combinations thereof may be added. If desired, surfactants may also be added to the formulation.

In a preferred aspect, the invention provides a dilute 4-hydroxyl pentanoic acid lactone solution comprising the 4-hydroxyl pentanoic acid lactone in a concentration of about 14 mg/ml, a buffer or other additive to maintain the pH between about 2.2 and 7, at least one preservative, at least one flavoring agent, and at least one coloring agent. The preferred flavoring agents include menthol, menthone, and carvone. More preferably, the pH is between about 2.2 and 5. Yet more preferably, the pH is between about 3 and 3.5. If desired, surfactants may also be added to the formulation.

In an aspect, the invention provides a dilute 4-hydroxyl pentanoic acid lactone solution comprising the 4-hydroxyl pentanoic acid lactone in a concentration of about 14 mg/ml, and optionally a buffer or other additive to maintain the pH between about 2.2 and 7. One or more preservatives, flavoring agents, coloring agents, or combinations thereof may be added. If desired, surfactants may also be added to the formulation.

In a preferred aspect, the invention provides a concentrated 4-hydroxyl pentanoic acid lactone solution comprising the 4-hydroxyl pentanoic acid lactone in a concentration exceeding 120 mg/ml, a buffer or other additive to maintain the pH between about 2.2 and 7, at least one preservative, at least one flavoring agent, and at least one coloring agent. The preferred flavoring agents include menthol, menthone, and carvone. More preferably, the pH is between about 2.2 and 5. Yet more preferably, the pH is between about 3 and 3.5. If desired, surfactants may also be added to the formulation.

In an aspect, the invention provides a concentrated 4-hydroxyl pentanoic acid lactone solution comprising the 4-hydroxyl pentanoic acid lactone in a concentration exceeding 120 mg/ml, and optionally a buffer or other additive to maintain the pH between about 2.2 and 7. One or more preservatives, flavoring agents, coloring agents, or combinations thereof may be added. If desired, surfactants may also be added to the formulation.

In an aspect, the invention provides a concentrated solution of the sodium salt of 4-hydroxyl pentanoic acid at a pH of about 2.2 to 11. More preferably, the pH is about 3.0 to 8.0. The solution comprises the sodium salt of 4-hydroxyl pentanoic acid in a concentration exceeding 170 mg/ml. A buffer or pH adjusting agent may be added to obtain the desired pH. A flavoring agent may be added. A coloring agent may be added.

In another aspect, the invention provides a concentrated 4-hydroxyl pentanoic acid lactone solution at a pH of about 2.2 to 11. More preferably, the pH is about 3.0 to 8.0. The solution comprises 4-hydroxyl pentanoic acid in a concentration exceeding 120 mg/ml. A buffer or pH adjusting agent may be added to obtain the desired pH. A flavoring agent may be added. A coloring agent may be added.

In another aspect, the invention provides a dilute solution of the sodium salt of 4-hydroxyl pentanoic acid at a pH of about 2.2 to 9.0. More preferably, the pH is about 3.0 to 8.0. The solution comprises the sodium salt of 4-hydroxyl pentanoic acid in a concentration of 12 to 40 mg/ml. A buffer or pH adjusting agent may be added to obtain the desired pH. A flavoring agent may be added. A coloring agent may be added.

In another aspect, the invention provides a dilute 4-hydroxyl pentanoic acid lactone solution at a pH of about 2.2 to 9.0. More preferably, the pH is about 3.0 to 8.0. The solution comprises 4-hydroxyl pentanoic acid lactone in a concentration of 8 to 30 mg/ml. A buffer or pH adjusting agent may be added to obtain the desired pH. A flavoring agent may be added. A coloring agent may be added.

The term “effervescent” refers to agents capable of producing gas or bubbles including but not limited to carbon dioxide when inserted into liquid environments. For instance, when added to a liquid, such as an aqueous solution, a mixture of at least one acid and at least one salt results in a chemical reaction that liberates carbon dioxide. In one aspect, both the acid and the salt may be in anhydrous form. Examples of acids suitable for use include, but are not limited to, tartaric acid, citric acid, fumaric acid, adipic acid, malic acid, oxalic acid, or sulfamic acid, either alone or in combination. In an aspect, the effervescent is prepared from citric acid or a combination of citric acid and tartaric acid. Examples of salts suitable for effervescents include but are not limited to the alkali metal salts. Sodium carbonate, calcium carbonate, magnesium carbonate, ammonium carbonate, potassium carbonate, sodium bicarbonate, calcium bicarbonate, sodium sesequicarbonate, sodium glycine carbonate, L-lysine carbonate, and arginine carbonate may all be employed. Filming agents impart a film to the surface of a liquid. Froth is considered to be foam or foam of lasting duration. The advantage of adding an effervescent or filing agent to a formulation of GHB; GBL; 1,4-butanediol; 4-HPA; and 4-HPA lactone is that it notifies an unsuspecting person that an agent has been added to his food or beverage.

In an aspect, filming, foaming, or frothing agents may be added to dilute or concentrated aqueous solutions of GHB; GBL; 1,4-butanediol; 4-HPA; 4-HPA lactone, or combinations thereof. Filming agents refer to substances which impart a film to the surface of a liquid. The pharmaceutical compositions may optionally contain one or more coloring agents, pH adjusting agents, flavoring agents, buffers or preservatives. Filming, foaming, or frothing agents may be selected from but are not limited to esters of pectic or pectinic acids wherein the alcohol component is derived from aliphatic alcohols with up to 34 carbon atoms which may be saturated or unsaturated, said alcohols being unsubstituted or substituted by one or more, especially two functional units selected from the group consisting of amino, hydroxy, mercapto, aldehydro, keto, carboxyl, hydrocarbyl- and dihydrocarbylamino, ether, ester, thioether, thioester, acetal, ketal, carbalkoxy, carbamide and carbamide groups substituted by one or two hydrocarbyl groups, and in which such aliphatic alcohols may be interrupted in the carbon atom chain by heteroatoms chosen from the group formed by oxygen, sulfur and nitrogen, and salts thereof.

In an aspect, the alcohols which totally or partially esterify the carboxyl groups in pectic or pectinic acid are aliphatic alcohols with a maximum of 12 carbon atoms. Further filming, foaming, and frothing agents may be found in U.S. Pat. No. 5,384,400. The membrane of the bubbles of a foaming agent may be comprised of a surfactant such as carbohydrates, polysaccharides, derivatized carbohydrates, such as fatty acid esters of sugars such as sucrose (including sucrose stearate), and proteinasceous surfactants including albumin. Alternatively, the membrane of the bubbles may be a solid or semi-solid, such as hardened, thickened, or denatured proteinaceous material (e.g. albumin), carbohydrates, and the like.

Surfactants may be used as foaming agents. Surfactants suitable for use in the present invention include any compound or composition that aids in the formation or maintenance of the bubble membrane by forming a layer at the interface between the liquid and gas phases. The foaming agent or surfactant may comprise a single compound or a combination of compounds, such as the case of co-surfactants. Example of suitable surfactants or foaming agents include: block copolymers of polyoxypropylene polyoxyethylene, sugar esters, fatty alcohols, aliphatic amine oxides, hyaluronic acid aliphatic esters, hyaluronic acid aliphatic ester salts, dodecyl poly(ethyleneoxy)ethanol, nonylphenoxy poly(ethyleneoxy)ethanol, hydroxylethyl starch, hydroxylethyl starch fatty acid esters, dextrans, dextran fatty acid esters, sorbitol, sorbitol fatty acid esters, gelatin, serum albumins, or combinations thereof.

Foaming agents may be selected from phospholipids, nonionic surfactants, neutral or anionic surfactants, fluorinated surfactants, which can be neutral or anionic, or combinations of such emulsifying or foaming agents. Nonionic surfactants include polyoxyethylene-polyoxypropylene copolymers, polyoxyethylene fatty acid esters, such as polyoxyethylene stearates, polyoxyethylene fatty alcohol ethers, polyoxyethylated sorbitan fatty acid esters, glycerol polyethylene glycol oxystearate, glycerol polyethylene glycol ricinoleate, ethoxylated soybean sterols, ethoxylated castor oils, and hydrogenated derivatives thereof, and cholesterol. Anionic surfactants, particularly fatty acids (or their salts) having 12 to 24 carbon atoms, may also be used. One example of a suitable anionic surfactant is oleic acid, or its salt, sodium oleate. It will be appreciated that a wide variety of surfactants can be used. Indeed, virtually any non-toxic surfactant or foaming agent capable of facilitating formation of bubbles can be used in the present invention.

In certain other embodiments, the method of preparing the pharmaceutical composition comprises admixing GHB; GBL; 1,4-butanediol; 4-HPA; and/or 4-HPA lactone, one or more coloring agents, one or more flavoring agents, a pH adjusting or buffering agent, and an aqueous and/or organic medium soon before administration to a patient suspected of having a condition responsive to GHB; GBL; 1,4-butanediol; 4-HPA; and/or 4-HPA lactone.

The invention also provides a method of treating any disorder responsive to GHB, comprising administering to a patient suspected of having such a condition a therapeutic amount of a pharmaceutical composition comprising GHB (e.g. 0.5-6 grams) in a dilute aqueous solution and/or aqueous medium.

Conditions treatable with GHB; GBL; 1,4-butanediol; 4-HPA; and 4-HPA lactone include acquired resistance to GABAnergic agents, insomnia, anxiety disorders, obsessive compulsive symptoms, narcolepsy major, narcolepsy minor, restless legs syndrome, periodic limb movement disorder, subclinical restless legs syndrome or periodic limb movement disorder, mania, hypomania, bipolar disorder, cyclothymia, and depression.

In one aspect, the invention provides aqueous suspensions of GHB; GBL; 1,4-butanediol; 4-HPA; and/or 4-HPA lactone in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents, such as a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, one or more coloring agents, one or more flavoring agents, and one or more sweetening agents, such as sucrose or saccharin.

In another aspect, the invention provides oily suspensions comprising GHB; GBL; 1,4-butanediol; 4-HPA; and/or 4-HPA lactone suspended in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Pharmaceutical compositions of the invention may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain buffering agents, additives, sweetening and/or flavoring agents.

Syrups and elixirs may be formulated with sweetening agents, for example glycerol, propylene glycol, sorbitol, glucose or sucrose. Such formulations may also contain a demulcent, a preservative, flavoring and/or coloring agents.

An advantage of solid state formulations of GHB; GBL; 1,4-butanediol; 4-HPA; and 4-HPA lactone is that shipping and storage costs are greatly reduced compared to solutions. In an aspect, the invention comprises GHB; GBL; 1,4-butanediol; 4-HPA; 4-HPA lactone, or combinations thereof in a solid or semi-solid state such as a tablet, capsule, block, wafer, or pill combined with a distinctive flavoring agent and/or coloring agent and/or effervescent. Optionally a foaming, frothing, or filming agent may be added. Preservative may be added. Additional acidifying or alkanalizing agents may be added such that the pH of a solution produced by dissolving the solid or semisolid formulations fall into a target range. The preferred pharmaceutical agents for solid formulations are the sodium salt of gamma-hydroxyl butyric acid and the sodium salt of 4-hydroxyl pentanoic acid. Flavoring agents, coloring agents, preservatives, and filming, foaming, and frothing agents are discussed above.

In an aspect, the solid or semi-solid state formulation of GHB; GBL; 1,4-butanediol; 4-HPA; or 4-HPA lactone may also contain one or more agents independently selected from Class III-VI GABAnergic agents (described later) for the treatment of diseases or conditions which are responsive to GABAnergic agents. In an aspect, the dose of the sodium salt of gamma-hydroxyl butyric acid is 0.25 to 7 grams. In a preferred aspect, the dose of the sodium salt of gamma-hydroxyl butyric acid is about 1 to 5 grams. A patient should be instructed to dissolve the pill in an aqueous medium such that the concentration of NaGHB is sufficiently dilute to avoid alimentary tract toxicity.

In a preferred aspect, the concentration of the solution resulting from dissolving a solid or semisolid formulation of NaGHB is about 50 to 110 mg/ml NaGHB. In a preferred aspect, the resultant pH of the solution derived from dissolving the salt of gamma-hydroxyl butyric acid in aqueous solution is about 2.2-9.0 (more preferably about 3-8). If the desired pharmaceutical agent to be delivered is GBL, the target pH is acidic and less than 6. If the desired pharmaceutical agent to be delivered is GHB, the target pH is above 6. However, mixtures which are combinations of GHB and GBL may be used, in which case the target pH is about 6. In a preferred aspect, the pH is about 3 to 8. Solid state and semi-solid state compositions and solutions which are mixtures of GHB; GBL; and/or 1,4-butanediol are encompassed in the invention.

In the most preferred aspect, the pH of the solution resulting from dissolving the solid or semi-solid state formulations is about 6.5 to 7.5. In an aspect, the invention is a pharmaceutical composition comprising one or more pharmaceutically acceptable salts of gamma-hydroxyl butyric acid in a solid or semi-solid state such as a tablet, capsule, block, or pill which also contains one or more substances which become emulsified or are insoluble when placed into aqueous medium, such that when placed into aqueous medium, emulsified or particulate matter is visibly detectable.

In an aspect, the invention comprises one or more pharmaceutically acceptable salts of gamma-hydroxyl butyric acid in a solid or semi-solid state such as a tablet, capsule, block, or pill combined with an effervescent and a foaming or frothing agent such that the foam or froth is increased by the bubble forming activity of the effervescent; optionally a distinctive flavoring agent, a coloring agent including but not limited to red, blue, or green coloring agents, and/or a preservative may be included.

In an aspect, the invention comprises one or more 4-hydroxyl C₅-C₁₀ alkanoic acids, salts thereof; the corresponding gamma lactones; and/or 1,4 C₅-C₁₀ alkanoic diols in a solid or semi-solid state such as a tablet, capsule, block, or pill. The preferred salts are the sodium salts.

In a preferred aspect, the invention comprises one or more pharmaceutically acceptable salts of 4-hydroxyl pentanoic acid in a tablet, capsule, block, or pill combined with a distinctive flavoring agent, a coloring agent including but not limited to red, blue, or green coloring agents, optionally a preservative, optionally an effervescent, and optionally a foaming, frothing, or filming agent. In an aspect, the pill may also contain one or more further GABAnergic agents for the treatment of diseases or conditions which are responsive to GABAnergic agents, preferred agents being Class III, IV, or V agents. In an aspect, the dose of the sodium salt of 4-hydroxyl pentanoic acid is 0.25 to 8 grams. In a preferred aspect, the dose of the sodium salt of 4-hydroxyl pentanoic acid is about 1 to 5 grams. A patient should be instructed to dissolve the pill in an aqueous medium such that the concentration of the salt of pentanoic acid is sufficiently dilute to avoid alimentary tract toxicity.

In a preferred aspect, the resultant pH of the solution derived from dissolving the salt of 4-hydroxyl pentanoic acid in aqueous solution is about 2.2-9.0. In a preferred aspect, the pH is about 3 to 8. Solutions which are mixtures of salts of 4-hydroxyl pentanoic acid and 4-hydroxyl pentanoic acid lactone are encompassed in the invention. In the most preferred aspect, the pH of the solution is about 6.5 to 7.5.

In an aspect, the invention is a pharmaceutical composition comprising one or more pharmaceutically acceptable salts of 4-hydroxyl pentanoic acid in a solid or semi-solid state such as a tablet, capsule, block, or pill which also contains one or more substances which become emulsified or are insoluble when placed into aqueous medium, such that when placed into aqueous medium, emulsified or particulate matter is visibly detectable.

In an aspect, the invention comprises one or more Class II GABAnergic agents in a solid or semi-solid state such as a tablet, capsule, block, or pill combined with an effervescent and a foaming or frothing agent such that the foam or froth is increased by the bubble forming activity of the effervescent; optionally a distinctive flavoring agent, a coloring agent including but not limited to red, blue, or green coloring agents, and/or a preservative may be included. The preferred Class II GABAnergic agents for solid or semisolid formulations are salts of 4-hydroxyl pentanoic acid, sodium salt preferred.

The formulations of the inventions of solid or semi-solid gamma-hydroxyl butyric acid or salts thereof and 4-hydroxyl pentanoic acid or salts thereof may contain at least one flavoring agent. While any flavoring agent may be used, the flavoring agent should be strong, i.e., not easily masked by the taste of soda, beer, or other alcoholic beverages. Preferred flavoring agents include menthol, menthone, vanillin, benzaldehyde, methyl butyrate, ethyl acetate, cinnamic aldehyde, or combinations thereof. In an aspect, the preferred flavor is minty and the preferred flavoring agents are menthol, menthone, or combinations thereof. Carvone, spearmint, and essential oil of spearmint may also be used to produce a minty taste. Tart is also a preferred taste. Preferred flavoring agents to produce a tart taste include citric acid, tartaric acid, and salts thereof.

If desired, one or more sweenteners, coloring agents, buffering agents, or preservatives may be added. Preferred preservatives for solid or semi-solid formulations include sorbic acid and salts thereof.

Compositions intended for oral use may be prepared according to any method known to the art for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

In a preferred aspect, the solubility of solid or semi-solid formulations of gamma-hydroxyl butyric acid, salts thereof; GBL; 1,4-butanediol; 4-hydroxyl pentanoic acid or salts thereof; or 4-hydroxyl pentanoic acid lactone are sufficiently low that dissolving the formulation results in a concentration of components that is non-toxic. In a preferred aspect, the concentration of the sodium salt of gamma-hydroxyl butyric acid; GBL; 1,4-butanediol; the sodium salt of 4-hydroxyl pentanoic acid; or 4-hydroxyl pentanoic acid lactone is about 200 mg/ml or less. In a yet more preferred aspect, the concentration of the sodium salt of gamma-hydroxyl butyric acid; GBL; 1,4-butanediol; the sodium salt of 4-hydroxyl pentanoic acid; or 4-hydroxyl pentanoic acid lactone is about 100 mg/ml or less.

It will be understood that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

### DETAILED DESCRIPTION OF TREATMENT OF ARG, ARG TYPE I AND ARG TYPE II

The inventor has surprisingly found that compositions comprising GABAnergic agents from Classes I-VI as described below are useful in the treatment of ARG. Similarly, methods of administering combinations of GABAnergic agents from Classes I-VI are useful in treating ARG. The method of treating ARG type I and ARG type II is the same.

The preferred route of administration of each Class of compound is oral.

In an aspect, a patient with acquired resistance to GABAnergic agents (ARG) may be treated by administering therapeutically effective amounts of two or more GABAnergic agents prior to sleep, where the agents are independently selected from Class I agents, which are alcoholic beverages, Class II GABAnergic agents, in which Class II agents are gamma-hydroxyl butyric acid or pharmaceutically acceptable salts thereof; gamma-butyryl lactone; 1,4-butanediol; 4-hydroxyl pentanoic acid or pharmaceutically salts thereof; 4-hydroxyl pentanoic acid lactone; or derivatives of gamma-aminobutyric acid which bind the GABA receptor or are transformed into agents which bind the GABA receptor; Class III GABAnergic agents, which are benzodiazepines, Class IV GABAnergic agents, which bind subunits or configurations of the GABA receptor more specifically than benzodiazepines, Class V GABAnergic agents, which are structural analogs of gamma-amino butyric acid which do not bind the GABA receptor, and Class VI GABAnergic agents, which are barbiturates.

Class I GABAnergic agents are alcoholic beverages including but not limited to wine, red wine, dark beer, white whine, beer, ale, malt liquor, liquor, vodka, gin, rum, whiskey, gin, bourbon, scotch, tequila, champagne, liqueurs, triple sec, martinis, vermouth, brandy, armagnac, cocktails, absinthe, amaretto, cordials, gimlet, schnapps, and spirits. All alcoholic beverages are Class I GABAnergic agents. All mixtures of alcoholic beverages are Class I GABAnergic agents. All alcoholic beverages are useful in the treatment of ARG regardless of the concentration of ethanol. Alcoholic beverages with higher concentrations of ethanol are consumed in lesser volumes than alcoholic beverages with lower concentrations for the treatment of ARG. When alcoholic beverages are used for medicinal purposes, red wine is the preferred agent, as it contains salutary substances other than ethanol. On occasion doses as high as 2000 ml of red wine may be required. If the Class I agent is red wine, the volume is about 40-2000 ml per day, and if the Class I agent is beer is about 300 to five thousand milliliters. The Class I agent(s) may be administered at any point during the day although in a preferred aspect are administered predominantly prior to sleep. Should the patient be unable to drink such large volumes, about 50 to about 750 ml of red wine may be mixed with about 10 to 400 ml of liquor. If carbonated Class I GABAnergic agents such as beer are administered, and if the carbonation produces gastrointestinal symptoms, the agent may be decarbonated by stirring rapidly and decanting the foam into another vessel. Alcohol (ethanol) is a Class I GABAnergic agent. The preferred route of administration is oral, although ethanol solutions may be delivered intravenously.

Ethanol (alcohol) binds the GABA receptor. Alcohol is well-known as a respiratory depressant and can aggravate OSA. However, many patients with undiagnosed OSA tolerate alcoholic beverages on a nightly basis prior to sleep. Patients with OSA who have conditions that predispose them to cardiac arrhythmias should avoid excessive alcohol consumption prior to sleep. Such patients include OSA patients with known cardiac arrhythmias such as atrial fibrillation, supraventricular tachycardia, ventricular tachycardia, and patients with cardiomyopathy.

Alcohol is typically disruptive to sleep in patients who have no sleep disorders. A twenty year old person, after consuming 3 to 4 beers may have disrupted sleep, early morning awakening, and hangover. However, after developing ARG later in life alcoholic beverages, especially red wine, may be beneficial to her sleep.

Class II GABAnergic agents include gamma-amino butyric acid, or pharmaceutically acceptable salts thereof; gamma-hydroxyl butyric acid, or pharmaceutically acceptable salts thereof, preferably the sodium salt; 1,4-butanediol; and gamma-butyryl lactone (GBL). The sodium salt of gamma-hydroxyl butyric acid (GHB) is the preferred agent. GBL and 1,4-butanediol are believed to convert into GHB in vivo. Typically, a starting dose of NaGHB is about 2 grams prior to sleep, slowly titrated upwards over weeks to months as needed. A high dose for the treatment of narcolepsy would be 5.25 grams prior to sleep. Further Class II GABAnergic agents include 4-hydroxybutanal, succinic semialdehyde, succinic acid, chemical modifications of GHB including trans-4-hydroxycrotonic acid, 4-chlorobutyric acid, 4-methoxybutyric acid, N-benzylamide of alpha-(benzylamine)-gamma-hydroxyl butyric acid, N-(o-chlorobenzyl)-amide of alpha-(benzylamine)-gamma-hydroxyl butyric acid, alpha-(4-Phenylpiperazine)-gamma-hydroxybutyric acid, methyl 4-acetoxybutanoate, and ethyl 4-acetoxybutanoate. Wine acids such as tartaric acid, malic acid, fumaric acid, and salts and isomers thereof are Class II GABAnergic agents. Wine contains significant quantities of acids. Further Class II GABAnergic agents include gamma-hydroxyl acid-ethanolamide and cyclic derivatives of GHB such as gamma-thiobutyrolactone and NSC-382 . . . (6,7,8,9 tetrahydro-5-[H]benzocyclohepten-5-ol-4-ylideneacetic acid.) Further Class II GABAnergic agents may be found in “Gamma-hydroxybutyric acid (GHB) and its Chemical Modifications: A review of the GHBergic system”; Anna Waszkielewicz, Jacek Bojarski, Polish Journal of Pharmacology; 2004, 56, 43-49, ISSN 1230-6002.

Class II GABAnergic agents include C₅-C₁₀ alkanoic carboxylic acids; salts thereof; the corresponding gamma lactones; 1,4 C₅-C₁₀ alkanoic diols; or combinations thereof, including R-isomers, S-isomers, and combinations thereof. In addition to GHB, GBL, and 1,4-butanediol, the preferred Class II agents include 4-hydroxyl pentanoic acid, salts thereof, and 4-hydroxyl pentanoic acid lactone. The preferred salts are the sodium salts.

Class II GABAnergic agents also include mono- or di-esters of GHB. GHB may be esterified at the carboxyl group, the C4 hydroxyl group, or both. The GHB esters of the invention have the following structure:

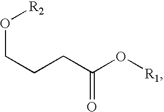

wherein
R₁ is H or C₁-C₆ alkyl; and
R₂ is H or C₁-C₆ alkanoyl; provided that at least one of R₁ and R₂ is not hydrogen.

The esters of GHB are formed by the reaction of an organic acid with GHB. Preparing the GHB esters may require the use of one or more protecting groups, which is known in the art.

Class II GABAnergic agents also include mono- or di-esters of 1,4-butanediol, which have the following structure:

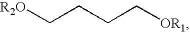

where R₁ and R₂ are independently H or C₁-C₆ alkanoyl; provided that at least one of R₁ and R₂ is not H.

The esters are formed by the reaction of organic acids with 1,4-butanediol. Preparing the 1,4-butanediol esters of the invention may require the use of one or more protecting groups, which is known in the art.

Class II GABAnergic agents further include mono- or di-esters of C₅-C₁₀ carboxylic acids, which have the following structure:

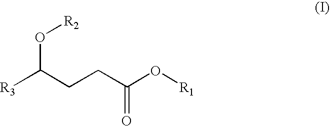

wherein
R₁ is H or C₁-C₆ alkyl;
R₂ is H or C₁-C₆ alkanoyl, and
R₃ is C₁-C₆ alkyl; provided that at least one of R₁ and R₂ is not hydrogen.

The esters are formed by the reaction of organic acids with the C₅-C₁₀ carboxylic acid. Preparing the mono- or di-esters of C₅-C₁₀ carboxylic acids of the invention may require the use of one or more protecting groups, which is known in the art.

Class II GABAnergic agents also include mono- or di-esters of 1,4 C₅-C₁₀ alkanoic diols, which have the following structure:

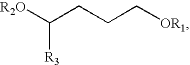

where
at least one of R₁ and R₂ is C₁-C₆ alkanoyl, while the other is H or C₁-C₆ alkanoyl; and
R₃ is C₁-C₆ alkyl.

Esters of 1,4 C₅-C₁₀ alkanoic diols are formed by the reaction of organic acids with 1,4 C₅-C₁₀ alkanoic diols. Preparing the mono- or di-esters of C₅-C₁₀ carboxylic acids of the invention may require the use of one or more protecting groups, which is known in the art.

Still more Class II GABAnergic agents may be found in United States patent applications 20020165224; 20050113366, and 20050250848, U.S. Pat. Nos. 4,599,355; 4,738,985; 5,840,331; 5,990,162; 5,380,937; 5,594,030; 6,623,730; 6,770,784; and 5,753,708, which, like every reference disclosed herein, are incorporated by reference in their entirety for all purposes.

Class III GABAnergic agents are considered to be benzodiazepines, such as alprazolam, clonazepam, diazepam, lorazepam, clorazepate, oxazepam, flurazepam, estalozam, triazolam, chlordiazepoxide, oxazepam, prazepam, quazepam, temazepam, and nitrazepam. Preferred Class III agents have short to moderate duration of action such that they stabilize sleep until the end of the sleep period but do not produce next day sedation. Alprazolam and clonazepam are among the preferred Class III agents. Class III agents have been reported to decrease stages 3 and 4 of sleep. However, the significance of this finding has been vastly exaggerated and Class III agents remain very useful medications.

Benzodiazepines are known to bind the GABA receptor. The amount of benzodiazepines required depends on metabolism, age, sex, height, weight, and individual variation.

In one aspect doses of Class III GABAnergic agents are alprazolam 0.125 mg to 4 mg and/or clonazepam 0.125 to 4 mg and/or diazepam about 5 to 20 mg. In another aspect, doses of Class III agents when combined with Class II agents are alprazolam about 3 mg or clonazepam about 3 mg.

In an aspect, patients with ARG may be treated with higher doses of Class III agents than are typically used for other conditions. The upper limit typically advised for alprazolam is 2 mg, for clonazepam is 2 mg, and for lorazepam is 4 mg. However, patients with ARG may be treated with higher doses such as 4 mg alprazolam or 4 mg clonazepam or lorazepam 6 mg.

Class IV GABAnergic agents are compounds that bind one or more subunits or configurations of the GABA receptor more specifically than benzodiazepines, or which bind subunits of configurations of the GABA receptor which are localized to particular areas of the brain. In a preferred aspect, when the class IV GABAnergic agents binds to the GABA_{A} receptor, it does so more specifically than benzodiazepines. Class IV agents include but are not limited zolpidem, the zaleplon, zopiclone, and eszopiclone, or baclofen or similar agents. The preferred GABAnergic agents are zolpidem, zaleplon, zopiclone, and eszopiclone. These medications are similar to benzodiazepines and show selectivity for the GABA_{A} receptor and for one or more subunits or configurations of the GABA receptor.

Baclofen may bind the GABA-B receptor, but in therapeutic doses its sleep-inducing effects are far less than those of GHB. Baclofen is used as a muscle relaxant, although it may be of some effect in controlling restless legs syndrome or periodic limb movement disorder.

Class V GABAnergic agents do not bind the GABA receptor but have inhibitory neurologic effects or GABAnergic effects based on structural similarity to GABA. Class V agents include but are not limited to gabapentin, pregabalin, and tiagabine. Preferred Class V agents are gabapentin and/or pregabalin.

Gabapentin is a structural analog of GABA with unknown mechanism. It does not bind the GABA receptor. Gabapentin may bind the alpha.sub.2-delta site, an auxiliary subunit of voltage-gated calcium channels. It is highly useful as therapy for a variety of conditions including neuropathic pain, restless legs syndrome (RLS), periodic limb movement disorder (PLMD), and as adjunctive therapy for seizure disorders. Typical doses uses are 100 to 800 mg prior to sleep for RLS/PLMD. Doses as high as 1600 mg three times per day have been used for neuropathic pain, although many patients experience sedation and slurred speech at such high doses. In an aspect of the invention, doses of gabapentin for the treatment of ARG when combined with one or more GABAnergic agents are about 100 to 1600 mg. In a preferred aspect, the dose is 100 to 500 mg.

Pregabalin is a structural derivative of GABA which is not believed to bind the GABA receptor. Pregabalin, based on its similarity with gabapentin, may bind the alpha.sub.2-delta site. In cultured neurons pregabalin increases the density of GABA transporter protein and increases the rate of functional GABA transport.

Tiagabine is believed to bind recognition sites associated with the GABA uptake carrier, blocking GABA uptake into presynaptic neurons, permitting more GABA to be available for receptor binding on the surfaces of past-synaptic cells. The use of the term tiagabine is understood to encompass tiagabine hydrochloride or other pharmaceutically acceptable salts.

The currently marketed Class V agents are the weakest of the Class I-V agents in treating ARG. Nevertheless, in combination with other GABAnergic agents they are useful.

Class VI GABAnergic agents are barbiturates, including but not limited to barbiturate nucleus, pentobarbital, secobarbital, phenobarbital, amobarbital, aprobarbital, butabarbital, mephobarbital, and primidone. Although useful in the treatment of ARG when combined with other agents described herein, the preferred agents are II, III, IV, and V agents. Preferred Class VI agents are phenobarbital and primidone.

The methods of the invention contemplate the use of either standard or slow release formulations of the agents from the Classes II, III, IV, V, or VI. In a preferred aspect, the Class II agents are in solution for oral consumption and are absorbed quickly into the bloodstream to decrease fatigue, alleviate the symptoms of ARG, or induce sleep quickly. In a preferred aspect, the Class II agents are in aqueous solution for oral consumption.

In an aspect, the methods of the invention further compromise administering a combination of an opiate or opioid, such as propoxyphene, hydrocodone, oxycodone, morphine, hydromorphone, oxymorphone, fentanyl, levorphanol, meperidine, methadone, codeine, dihydrocodeine, butorphanol, pentazocine, or buprenorphine, and various combinations of Class I, II, III, IV, V, and/or VI GABAnergic agents for the treatment of ARG. Preferred opiates include, but are not limited to oxycodone, and propoxyphene (including the propoxyphene naphthylic acid salt). Patients with sleep apnea may require dosage reductions. GABAnergic agents when combined with opiates or opioids may increase respiratory depression.

To obtain the final regimen which is most therapeutic for an individual patient with ARG, one or more GABAnergic agents may be initiated and subsequent GABAnergic agents may be added or removed in a variety of orders. The order of addition of medication is highly variable.

In an aspect, a patient with ARG may be treated with Class II GABAnergic agents such as gamma-hydroxyl butyric acid or pharmaceutically acceptable salts thereof; gamma-butyryl lactone; 1,4-butanediol; 4-hydroxyl pentanoic acid or pharmaceutically acceptable salts thereof; 4-hydroxyl pentanoic acid lactone, or combinations thereof prior to sleep. A patient with ARG may be treated with the sodium salt of gamma-hydroxyl butyric acid.

In another aspect, a patient may be treated prior to sleep with about 0.004 to 0.05 moles of medication in which the medication is a combination of GHB; GBL; and/or 1,4-butanediol. In another aspect, a patient may be treated prior to sleep with about 0.004 to 0.06 moles of medication in which the medication is a combination of GHB; GBL; 1,4-butanediol; 4-HPA; 4-HPA lactone; or combinations thereof. In another aspect, a patient may be treated prior to sleep with about 0.004 to 0.08 moles of medication in which the medication is a combination of 4-HPA; 4-HPA lactone; and/or 1,4-pentane diol. In an aspect, the invention comprises a method of treating patients with ARG type I with one or more GABAnergic agents.

In a preferred aspect, the Class I or II GABAnergic agent(s) are consumed with or soon after other GABAnergic agents soon prior to sleep or to induce sleep. A patient with ARG may be treated with Class I or II agents during waking hours to induce sleep or in lesser doses to decrease fatigue. Preferred Class II agents for waking hours include pharmaceutically acceptable salts of gamma-hydroxyl butyrate such as the sodium salt; gamma-butyryl lactone; 1,4-butanediol; 4-HPA; 4-HPA lactone; or combinations thereof.

In another aspect, a patient is treated with about 0.25 to 5.25 grams of the sodium salt of gamma-hydroxyl butyric acid during waking hours. The dose required during waking hours is highly variable between patients and must be empirically determined for each patient. Doses should be started low and increased slowly. In another aspect, a patient is treated with about 0.17 to 3.75 grams of GBL or about 0.17 to 3.75 grams of 1,4-butanediol. Likewise, the dose required during waking hours is highly variable between patients and must be empirically determined for each patient. Doses should be started low and increased slowly.

In another aspect, a patient may be treated during waking hours with about 0.004 to 0.04 moles of medication in which the medication is a combination of GHB; GBL; and/or 1,4-butanediol. In another aspect, a patient may be treated during waking hours with about 0.004 to 0.04 moles of medication in which the medication is a combination of GHB; GBL; and/or 1,4-butanediol. In another aspect, a patient may be treated during waking hours with about 0.004 to 0.08 moles of medication in which the medication is a combination of 4-HPA; 4-HPA lactone; and/or 1,4-pentane diol.

In an aspect, the invention is a method of treating ARG, ARG type I or ARG type II comprising administering one or more Class II GABAnergic agents 1 to 5 times per sleep period, the first dose prior to sleep and the subsequent doses administered periodically during the sleep period, whether the patients awakens spontaneously or by alarm. The preferred Class II agents are pharmaceutically acceptable salts of gamma-hydroxyl butyrate such as the sodium salt; gamma-butyryl lactone; 1,4-butanediol; 4-HPA; 4-HPA lactone; or combinations thereof. In a preferred aspect, the Class II agent(s) are administered 2 to three times per sleep period, the first dose prior to sleep and the subsequent doses periodically during the sleep period. The most preferred time interval between the first and second doses is about 2 to 6 hours.

A patient with ARG may be started on GHB; GBL; 1,4-butanediol; 4-HPA; 4-HPA lactone; or 1,4-pentane diol prior to sleep. In an aspect, the starting dose of NaGHB is about 1.5 to 2 grams, the starting dose of GBL is about 1 to 2 grams, the starting dose of 1,4-butanediol is about 1 to 2 grams, the starting dose of 4-HPA is about 1 to 4 grams; and the starting dose of 4-HPA lactone is about 1 to 4 grams. In an aspect, the patient is started on any of these agents and the dose is slowly increased in increments of about 0.5 to 1 gram over weeks to months depending on side effects. If the patient does not have significant improvement in symptoms, at any time during the upward titration of the Class II GABAnergic agent, one or more additional GABAnergic agents are added. In a preferred aspect, a Class I, III, IV, or V agent is added. In an aspect, the preferred agents are Class III, W, and V. In a yet more preferred aspect, the Class III agent is alprazolam or clonazepam, the Class IV agent is zolpidem or zaleplon, and the Class V agent is gabapentin.

In another aspect, patients with ARG may be treated as follows: Initially, she is started on a GABAnergic agent at low to moderate dose. Zolipidem or another GABAnergic agent by itself is rarely effective in controlling symptoms. After a roughly two week trial of a Class I and/or a Class II and/or a Class III and/or a Class IV and/or a Class V agent, and if there is no reason to suspect more than mild obstructive sleep apnea, the patient is started on a Class II agent. In another aspect, a patient may be started on a Class I agent and/or Class II agent and/or a Class III agent and/or a Class IV agent and/or a Class V agent and/or a Class IV agent. In another aspect, a patient may be started on more than one agents from the same class optionally with agents from other classes of GABAnergic agents.

A typical starting dose of the sodium salt of gamma-hydroxyl butyrate would be 1.5 to 2 grams. Over a period of weeks to months the doses of medications may slowly be increased depending on side effects such as next day drowsiness, nausea or dizziness, headache, enuresis, or dysphoria. Patients should not operate motor vehicles or machinery when drowsy, sleepy, or intoxicated. Strong hypnotics may be inappropriate for patients with small children at home who require attention. If a patient is treated with a concentrated form of a GHB; GBL; or 1,4-butanediol, the sleep-inducing effects at appropriate dosages are very strong and indicate that the patient must take Class III, IV, or V agents prior to, concurrently, or soon after consuming the Class II agents.

Concentrated formulations GHB; GBL; and 1,4-butanediol are typically consumed rapidly over seconds to minutes and have strong hypnotic properties when used in therapeutic dosages. Patients may be treated with a Class I agent and/or a Class II agent and/or a Class III agent and/or a Class IV agent and/or a Class V agent and/or a Class VI agent. The preferred agents are Classes I, II, III, IV, and V agents. Patients may be treated with a Class I and/or a Class II agent and/or a Class III agent and/or a Class IV agent and/or a Class V agent and/or a Class VI agent.

Ingestion of Class III, IV, and V agents subsequent to GHB; GBL; and 1,4-butanediol is effective but less preferred. The preferred agents are Classes I, II, III, IV, and V agents. Class VI agents are effective but less preferred because of next day sedation.

In another aspect, patients may be treated with a combination one or more Class II agents and/or one or more Class III agents and/or one or more Class IV agents and/or one or more Class V agents and/or one or more Class VI agents.

Which GABAminergic agent(s) administered prior to sleep at the initiation of treatment is highly variable. A Class I, II, III, IV, V, or VI agent may be used for initiation of therapy. Medications may be co-administered at the initiation of therapy.

Although ingestion of multiple GABAnergic agents, especially in large doses, is effective in overriding delayed sleep phase syndrome, the preferred method of treating patients with co-existing ARG and delayed sleep phase syndrome or advanced sleep phase syndrome is to give the GABAnergic agents at the patient's natural bedtime hour and allow the patient to sleep until her natural waking hour. Attempts to override the patient's natural circadian sleep/wake cycle produces less restful sleep. Often the patient should alter work schedules to allow her to sleep at a later hour then others if it her her natural tendency. Four hours of sleep at a patient's natural sleep time is often more restful than eight hours at another time of day. Some evidence suggests that obesity in The United States is due to sleep deficiency and sleeping at abnormal hours. Much of childhood obesity is due to lack of sleep. However, because cognitive behavioral therapy is generally ineffective in treating delayed sleep phase syndrome, treatment with multiple GABAnergic agents is occasionally warranted in patients who cannot alter their work schedules.

In an aspect, a patient with ARG is treated with a Class II agent combined with a Class III agent prior to sleep. In a preferred aspect, the Class II agent is gamma-hydroxyl butyric acid or a pharmaceutically acceptable salt thereof; GBL; 1,4-butanediol; 4-hydroxyl pentanoic acid or pharmaceutically slats thereof; 4-hydroxyl pentanoic acid lactone; and the class III agent is alprazolam, clonazepam, triazolam, lorazepam, or combinations thereof. A Class I agent may be used in place of or in addition to a Class II agent; the preferred Class I agent is red wine. If the Class I agent is red wine, the volume is about 40-2000 ml per day, and if the Class I agent is beer is about 300 to five thousand milliliters. The Class I agent may be administered throughout the day although in a preferred apsect is administered predominantly prior to sleep.

In an aspect, a patient with ARG is treated with a Class II agent combined with a class III agent prior to sleep. In another aspect, a patient consumes about 1.0 to 6.0 grams of the Class II agent the sodium salt of gamma-hydroxyl butyric acid and about 0.25 to 4 mg of the Class III agent alprazolam prior to sleep. In another aspect, a patient consumes about 1.0 to 6.0 grams of the sodium salt of gamma-hydroxyl butyric acid and about 0.25 to 4 mg clonazepam prior to sleep. Alternatively, about 1 to 6 grams of the Class II agent GBL or about 1 to 8 grams of the Class II agent the sodium salt of 4-hydroxyl pentanoic acid or 4-hydroxyl pentanoic acid lactone may be combined with Class III agents such as alprazolam, clonazepam, triazolam, or lorazepam. A Class I agent may be used in place of or in addition to a Class II agent; the preferred Class I agent is red wine. If the Class I agent is red wine, the volume is about 40-2000 ml per day, and if the Class I agent is beer is about 300 to five thousand milliliters. The Class I agent may be administered throughout the day although in a preferred apsect is administered predominantly prior to sleep.

In an aspect, a patient is treated with a Class II agent combined with a Class IV agent prior to sleep; furthermore the Class II agent is gamma-hydroxyl butyric acid or a pharmaceutically acceptable salt thereof; GBL; 1,4-butanediol; 4-hydroxyl pentanoic acid or salts thereof; 4-hydroxyl pentanoic acid lactone; or combinations thereof; and the class IV agent is zolpidem. A Class I agent may be used in place of or in addition to a Class II agent; the preferred Class I agent is red wine. If the Class I agent is red wine, the volume is about 40-2000 ml per day, and if the Class I agent is beer is about 300 to five thousand milliliters. The Class I agent may be administered throughout the day although in a preferred apsect is administered predominantly prior to sleep.

In an aspect, a patient with ARG is treated with a combination of a Class II agent, a Class III agent, and a Class IV agent prior to sleep; furthermore the Class II agent is gamma-hydroxyl butyric acid or a pharmaceutically acceptable salt thereof; GBL; 1,4-butanediol; 4-hydroxyl pentanoic acid or pharmaceutical salts thereof; 4-hydroxyl pentanoic acid lactone; or combinations thereof; the class III agent is alprazolam, clonazepam, triazolam, lorazepam or combinations thereof; and furthermore the Class IV agent is zolpidem, zaleplon, zopliclone, eszoplicone, or combinations thereof. In a preferred aspect the Class IV agent is zolpidem. A Class I agent may be used in place of or in addition to a Class II agent; the preferred Class I agent is red wine.

In an aspect, a patient with ARG may be treated with a Class II agent combined with a Class III agent and a Class IV agent and a Class V agent prior to sleep; the Class II agent is gamma-hydroxyl butyric acid or a pharmaceutically acceptable salt thereof; GBL; 1,4-butanediol; 4-hydroxyl pentanoic acid or pharmaceutically salts thereof; 4-hydroxyl pentanoic acid lactone; or combinations thereof; the Class III agent is alprazolam, clonazepam, triazolam, lorazepam, or combinations thereof; the Class IV agent is zolpidem, zaleplon, zopliclone, eszoplicone, or combinations thereof; the Class V agent is gabapentin. In a preferred aspect the Class IV agent is zolpidem. A Class I agent may be used in place of or in addition to a Class II agent; the preferred Class I agent is red wine.

In an aspect, a patient with ARG is treated with multiple GABAnergic agents prior to sleep in addition to GABAminergic agent(s) selected from Classes I, II, III, IV, V, and VI agents during waking hours. The preferred agents for use during waking hours are Class I and II agents. The preferred Class I agent is red wine. In a preferred aspect the Class II agent is GHB; GBL; 1,4-butanediol; 4-HPA; 4-HPA lactone, or combinations thereof. In a preferred aspect the salts of GHB and 4-HPA are the sodium salts.

In an aspect, a patient with acquired resistance to GABAnergic agents (ARG) is treated with a combination of one or more Class II agents and one or more class III agents and one or more Class IV agents and one or more Class V agents prior to sleep. A Class I agent may be used in place of or in addition to a Class II agent; the preferred Class I agent is red wine.

In another aspect, the invention is a method of treating a patient with acquired resistance to GABAnergic agents, said method comprising administering a therapeutically effective combination of Class I-V agents to a patient in need of such treatment. The preferred Class I agent is red wine. In a preferred aspect the Class II agents are gamma-hydroxyl butyric acid or pharmaceutically acceptable salts thereof; gamma-butyryl lactone; 1,4-butanediol; 4-hydroxyl pentanoic acid or pharmaceutically acceptable salts thereof; 4-hydroxyl pentanoic acid lactone, or combinations thereof; the Class III GABAnergic agents are alprazolam, clonazepam, triazolam, lorazepam, or combinations thereof; the Class IV GABAnergic agent is zolpidem; and the Class V GABAnergic agent is gabapentin, to a patient in need of such treatment. In the most preferred aspect the Class II agents are gamma-hydroxyl butyric acid or pharmaceutically acceptable salts thereof; gamma-butyryl lactone; 1,4-butanediol; 4-hydroxyl pentanoic acid or pharmaceutical salts thereof; 4-hydroxyl pentanoic acid lactone; or combinations thereof; the Class III agent is alprazolam, the Class IV agent is zolpidem, and the Class V agent is gabapentin.

Most patients with ARG require GABAnergic agents prior to sleep and do not require further GABAnergic agents prior to about twelve hours after sleep onset. However, in another aspect, a patient with ARG is treated with two or more GABAnergic agents prior to sleep which is regarded as the first dose and one or more GABAnergic agents about two to eight hours after sleep onset which is regarded as the second dose. The first dose may be a Class I, II, III, IV, V, or VI agent or combinations thereof. The second dose may be a Class I, II, III, IV, V, or VI agent or combinations thereof. Preferred agents for the first dose include Class II agents such as GHB; GBL; 1,4-butanediol; 4-HPA; 4-HPA lactone; Class III agents such as alprazolam, clonazepam, triazolam, and lorazepam; Class IV agents such as zolpidem and zaleplon; and Class V agents such as gabapentin. Preferred agents for the second dose include Class II agents such as GHB; GBL; 1,4-butanediol; 4-HPA; and 4-HPA lactone; Class III agents such as alprazolam and clonazepam; Class IV agents such as zolpidem and zaleplon; and Class V agents such as gabapentin. In another aspect, the second dose is given about six hours after sleep onset. In another aspect, opiates are consumed as adjunctive therapy with the first and/or second dose. In another aspect, the opiate is oxycodone or propoxyphene.

In an aspect, the invention comprises combining low to high doses of one or more Class III, one or more Class IV, one or more Class V, and/or one or more Class VI agents into the same pill for the treatment of ARG, insomnia, anxiety, narcolepsy, restless legs syndrome, periodic limb movement disorder, subclinical restless legs syndrome, subclinical periodic limb movement disorder, or other conditions responsive to GABAnergic agents. The preferred agents are Class III, IV, and V agents. In another aspect, the invention comprises combining low or high doses of one or more Class III, one or more Class IV, one or more Class V, one or more Class VI agents, and/or one or more opiates into the same pill for the treatment of ARG, fatigue, unrestful sleep, difficulty dealing with stress, arthralgias, myalgias, tense muscles, difficulty concentrating, impaired memory, insomnia, anxiety, depression, mania, hypomania, bipolar disorder, cylcothymia, sleepiness, narcolepsy major, narcolepsy minor, restless legs syndrome, periodic limb movement disorder, subclinical restless legs syndrome, subclinical periodic limb movement disorder, or other conditions responsive to GABAnergic agents or opiates. An opiate and/or opioid may also be included in the pill. GHB; GBL; 1,4-butanediol; 4-HPA; and 4-HPA lactone are useful for treating ARG associated with restless legs syndrome, periodic limb movement disorder of sleep, interstitial cystitis, irritable bowel syndrome, rheumatoid arthritis, lupus erythematosus, and multiple sclerosis.

In an aspect, low doses of Class III agents are considered to be alprazolam about 0.25 mg or clonazepam about 0.25 mg, low doses of Class IV agents are considered to be zolpidem about 2.5 mg or zaleplon about 2.5 mg, and low doses of gabapentin are considered to be about 25 to 100 mg, whereas high doses of Class III agents are considered to be alprazolam about 5 to 10 mg or clonazepam about 5 to 10 mg, high doses of Class IV agents are considered to be zolpidem about 20 to 70 mg or zaleplon about 20 to 100 mg, and high doses of Class V agents are considered to be gabapentin about 800 to 1600 mg. In a preferred aspect, the opiate is oxycodone and/or propoxyphene. In an aspect, low doses of opiates are considered to be propoxyphene about 10 mg or oxycodone about 2.5 mg, whereas high doses are considered to be propoxyphene about 200 mg or oxycodone about 120 mg.

Sleepiness during waking hours in the medical literature is often referred to as excessive daytime sleepiness or excessive daytime somnolence (EDS). EDS is frequently associated with fatigue. The multiple sleep latency test is not a test of daytime sleepiness; it is a test of sleep latency, which the time required to fall asleep. Sleepiness during waking hours is the desire to sleep, tendency to nap, or tendency to fall asleep unintentionally during waking hours. Numerous conditions may result in EDS including decreased sleep time for any reason such as decreased sleep time due to long work hours, stress, financial distress, pain, or insomnia. Numerous other conditions may result in EDS such as narcolepsy, narcolepsy major, narcolepsy minor, idiopathic hypersomnia, restless legs syndrome, subclinical restless legs syndrome, periodic limb movement disorder, subclinical periodic limb movement disorder, obstructive sleep apnea, central sleep apnea, and REM sleep behavior disorder.

GABAnergic agents are useful for various conditions associated with EDS or daytime fatigue. In an aspect, the invention comprises administering one or more GABAnergic agents independently selected from Class I-VI agents prior to sleep to a patient in need of such treatment. In an aspect, the preferred Class II agents are GHB; GBL; 1,4-butanediol; 4-HPA; and 4-HPA lactone. Stages 3 and 4 sleep have been documented to occur predominantly in the first cycles of sleep during the sleep period and to decrease in later cycles of sleep. Typically a patient goes through about 2 to 6 sleep cycles per sleep period. Some Class II agents administered prior to sleep are advantageous for inducing stages 3 and 4 sleep near the beginning of the sleep period other hypnotics such as Class III, IV, V, and VI agents administered prior to sleep will prolong the sleep period such that a second dose of Class II agent(s) is not required. In an aspect, the invention is a method of treating EDS or fatigue due to decreased sleep, insomnia, narcolepsy major, narcolepsy minor, idiopathic hypersomnia, restless legs syndrome, subclinical restless legs syndrome, periodic limb movement disorder, subclinical periodic limb movement disorder, obstructive sleep apnea, central sleep apnea, and REM sleep behavior disorder with one or more GABAnergic agents prior to sleep. In an aspect, the preferred Class II agents are GHB; GBL; 1,4-butanediol; 4-HPA; and/or 4-HPA lactone. In a more preferred aspect, one or more Class II agents are combined with one or more Class III, IV, or V agents prior to sleep.

Although at least some of the preferred Class II agents GHB; GBL; 1,4-butanediol; 4-HPA; and 4-HPA lactone increase stages 3/4 sleep, which tend to predominate near the beginning of the sleep period, the inventor has surprisingly found that administration of one or more GABAnergic agents prior to sleep which is regarded as the first dose and then administration one or more Class II agents about 2 to 8 hours after sleep onset is effective in the treatment of ARG. In an aspect, the first dose is a Class I agent and/or a Class III agent and/or a Class IV agent and/or a Class V agent. In an aspect, the Class I agent is red wine; the Class III agent is alprazolam or clonazepam; the Class IV agent is zolpidem or zaleplon; and the Class V agent is GABApentin. In an aspect, the second dose is GHB; GBL; 1,4-butanediol; 4-HPA; 4-HPA lactone, or combination thereof.

Preferred dual agent combination therapies for ARG, pain, insomnia, anxiety or anxiety disorders, bipolar disorder, mania, hypomania, cyclothymia, depression, alcoholism, restless legs syndrome, subclinical restless legs syndrome, periodic limb movement disorder, subclinical periodic limb movement disorder, attention deficit, hyperactivity, attention deficit hyperactivity disorder, narcolepsy major, and narcolepsy minor include a one or more Class III and one or more Class IV agents. Preferred Class III agents include alprazolam, clonazepam, triazolam, and lorazepam. In an aspect, the doses of are alprazolam are about 0.25-7 mg and clonazepam about 0.25-5 mg. The preferred Class IV agents are zolpidem about 2.5-60 mg and zaleplon about 2.5-60 mg. A Class III agent such as alprazolam, clonazepam, triazolam, or lorazepam may be combined with a Class V agent such as gabapentin or pregabalin. A Class IV agent such as zolipidem or zaleplon may be combined with may be combined with a Class V agent such as gabapentin or pregabalin. In a more preferred aspect, a dual agent combination is the Class III agent alprazolam about 0.25 to 7 mg and the Class IV agent zolpidem about 2.5 to 50 mg. In a yet more preferred aspect, the dual agent combination is the Class III agent alprazolam about 2 to 5 mg and the Class IV agent zolpidem about 10 to 15 mg. These dual agent combinations may be combined with one or more Class I or II agents.

Preferred triple agent combination therapies for ARG, pain, insomnia, anxiety or anxiety disorders, bipolar disorder, mania, hypomania, cyclothymia, depression, alcoholism, restless legs syndrome, subclinical restless legs syndrome, periodic limb movement disorder, subclinical periodic limb movement disorder, attention deficit, hyperactivity, attention deficit hyperactivity disorder, narcolepsy major, and narcolepsy minor include a one or more Class III and one or more Class IV agents and or one or more Class V agents. Preferred Class III agents include alprazolam, clonazepam, triazolam, and lorazepam. In an aspect, the doses of are alprazolam are about 0.25-7 mg and clonazepam about 0.25-5 mg. The preferred Class IV agents are zolpidem about 2.5-60 mg and zaleplon about 2.5-60 mg. The preferred Class V agents are gabapentin about 25 to 1000 mg and pregabalin about 25-1000 mg. In a more preferred aspect, the triple agent combination is the Class III agent alprazolam about 2 to 5 mg, the Class IV agent zolpidem about 10 to 15 mg, and gabapentin about 300 to 500 mg. These triple agent combinations may be combined with one or more Class I or II agents.

Many patients do not tolerate CPAP or Bi-level positive airway pressure (BiPAP) masks for the entire sleep period although there is a mild reduction in obstructive sleep apneic events for sleep periods when the mask is not applied if the patient has recently tolerated the mask such as at other times during the sleep period or during the prior sleep period. Patients who do not tolerate treatment for OSA or who have central sleep apnea may require lower doses of Class I or Class II GABAnergic agents. Excessive pressures of CPAP have been known to aggravate central sleep apnea. Some patients with conditions responsive to treatment with Class II GABAnergic agents who have co-existing obstructive sleep apnea should have the sleep apnea treated in addition to consuming GABAnergic agents. The preferred Class II agents are GHB; GBL; 1,4-butanediol; 4-HPA; 4-HPA lactone, or combinations thereof. In an aspect, the preferred Class II agents are 4-HPA and 4-HPA lactone to reduce respiratory depression and toxicity including nausea and vomiting.

Insomnia is sometimes associated with fatigue or sleepiness and sometimes not. In an aspect, the invention comprises a method of treating insomnia with one or more GABAnergic agents prior to sleep. The preferred Class I agent is red wine. The preferred Class II agents are GHB; GBL; 1,4-butanediol; 4-HPA; and 4-HPA lactone. In a preferred aspect, a preferred Class II GABAnergic agent is combined with one or more Class I, Class III, IV, and/or V agents. In a yet more preferred aspect, GHB; GBL; 1,4-butanediol; 4-HPA; and/or 4-HPA lactone are combined with alprazolam about 0.25-4 mg, zolpidem about 2.5-30 mg, and gabapentin about 100-500 mg prior to sleep for insomnia.

In an aspect, the GHB; GBL; 1,4-butanediol; 4-HPA; and/or 4-HPA lactone solution of the invention can also be used in conjunction with sleeping pill (such as zolpidem or zaleplon), a benzodiazepine (such as alprazolam, triazolam, clonazepam, temazepam, diazepam, or lorazepam) and gabapentin. In an aspect, he patient takes the sleeping pill, a benzodiazepine and the gabapentin and then approximately 20 minutes later, the GHB; GBL; 1,4-butanediol; 4-HPA; and/or 4-HPA lactone. As the effects of 4-HPA and 4-HPA lactone are less pronounced, in an aspect, the patient may take the 4-HPA or 4-HPA lactone prior to, concurrently, or subsequently to other GABAnergic agents. However, the order in which patients may take GABAnergic agents is highly variable.

In an aspect, the invention comprises a method of treating anxiety, anxiety disorders, or mania or hypomania with one or more GABAnergic agents prior to sleep. The preferred Class I agent is red wine. The preferred Class II agents are GHB; GBL; 1,4-butanediol; 4-HPA; and 4-HPA lactone. In a preferred aspect, a preferred Class II GABAnergic agent is combined with one or more Class I, Class III, IV, and/or V agents. In a yet more preferred aspect, GHB; GBL; 1,4-butanediol; 4-HPA; and/or 4-HPA lactone are combined with alprazolam about 0.25-4 mg, zolpidem about 2.5-30 mg, and gabapentin about 100-500 mg prior to sleep for insomnia. In a preferred aspect, the patient is a human. However, animals may also be treated.

In an aspect, the invention comprises a method of treating fatigue or excessive daytime sleepiness of undetermined etiology with one or more GABAnergic agents prior to sleep. The preferred Class I agent is red wine. The preferred Class II agents are GHB; GBL; 1,4-butanediol; 4-HPA; and 4-HPA lactone. In a preferred aspect, a preferred Class II GABAnergic agent is combined with one or more Class I, III, IV, and/or V agents. In a yet more preferred aspect, GHB; GBL; 1,4-butanediol; 4-HPA; and/or 4-HPA lactone are combined with alprazolam about 0.25-4 mg, zolpidem about 2.5-30 mg, and gabapentin about 100-500 mg prior to sleep for insomnia.

Whether Class II agents aggravate sleep apnea is unknown. However, Class II agents are expected to aggravate sleep apnea in some patients. The diagnosis of narcolepsy is controversial. The pathophysiology of how obstructive sleep apnea unmasks or aggravates narcolepsy in some patients is disclosed in this document. Currently some patients with both obstructive sleep apnea and narcolepsy are treated with GHB prior to sleep if they are under treatment for obstructive sleep apnea such as CPAP, BiPAP, or oral device. Patients with obstructive sleep apnea who do not have narcolepsy are not currently treated with GHB. However, physicians do not appreciate that the pressure of CPAP is significant, even at pressures as low as 5 to 6 cm water, and that the pressure itself can disrupt sleep, even if elimination or reduction of apneas improves sleep. Thus, the same treatment (CPAP) can both be helpful and deleterious to sleep. The inventor has identified a patient whose sleep was made to be less restful by chronic use of CPAP and whose sleep became more restful upon discontinuation of CPAP (CPAP-induced fatigue or sleepiness).

In an aspect, the invention comprises administering to a patient with obstructive sleep apnea who is treated with CPAP, BiPAP, and/or oral device one or more GABAnergic agents prior to sleep for the treatment of fatigue, EDS, hyperactivity, anxiety, restlessness, decreased concentration, depressions, bipolar disorder, mania, hypomania, other symptoms due to obstructive sleep apnea, or CPAP-induced fatigue or sleepiness. The preferred Class II GABAnergic agents are GHB; GBL; 1,4-butanediol; 4-HPA; 4-HPA, or combinations thereof.

GHB is known to increase stages 3 and 4 sleep, which naturally are more prevalent towards the beginning of the sleep period and decrease later in the sleep period. Administration of GHB soon after sleep such as 2.5 to 4 hours after sleep onset may be deleterious for sleep in some patients. Administration of GHB more than once per night is necessary in some patients but is inconvenient and may often be avoided by co-administering another GABAnergic agent prior to sleep. In an aspect, the invention is a method of treating conditions responsive to Class II GABAnergic agents which have already been listed including ARG, decreased sleep, insomnia, narcolepsy major, narcolepsy minor, idiopathic hypersomnia, restless legs syndrome, periodic limb movement disorder, obstructive sleep apnea, central sleep apnea, REM sleep behavior disorder, decreased stages 3 or 4 of sleep, anxiety or anxiety disorders, fatigue, and/or EDS comprising administering therapeutically effective amounts of two or more GABAnergic agents prior to sleep such that overall sleep quality is improved, fatigue is decreased, or such that fewer dosing occasions are necessary than if one GABAnergic agents were used alone.

In an aspect, said method is a method of treating patients suffering from conditions responsive to Class II GABAnergic including ARG, decreased sleep, insomnia, narcolepsy major, narcolepsy minor, idiopathic hypersomnia, restless legs syndrome, periodic limb movement disorder, obstructive sleep apnea, central sleep apnea, REM sleep behavior disorder, decreased stages 3 or 4 of sleep, anxiety or anxiety disorders, fatigue, and/or EDS comprising administering therapeutically effective amounts of two or more GABAnergic agents prior to sleep where each agent is independently selected from Class I GABAnergic agents, which are alcoholic beverages, Class II GABAnergic agents, in which Class II agents are GHB; GBL; 1,4-butanediol; 4-HPA; 4-HPA lactone; or combinations thereof; Class III GABAnergic agents, which are benzodiazepines, Class IV GABAnergic agents, which bind subunits or configurations of the GABA receptor more specifically than benzodiazepines, Class V GABAnergic agents, which are structural analogs of gamma-amino butyric acid which do not bind the GABA receptor, and Class VI GABAnergic agents, which are barbiturates, to a patient in need of such treatment. In a preferred aspect, the Class II GABAnergic agent is GHB; GBL; 1,4-butanediol; 4-HPA, and/or 4-HPA lactone. In a preferred aspect, one or more Class III-V agents are combined with a Class II agent. In a still more preferred aspect the Class II agent is GHB; GBL; 1,4-butanediol; 4-HPA; or 4-HPA lactone; the Class III agent is alprazolam, the Class IV agent is zolpidem, and the Class V agent is gabapentin.

ARG is often associated with other disorders the symptoms of which may improve upon treatment of co-existing ARG. ARG may co-exist with disorders such as restless legs syndrome, subclinical restless legs syndrome, periodic limb movement disorder of sleep, subclinical periodic limb movement disorder, obstructive sleep apnea, narcolepsy, interstitial cystitis, irritable bowel syndrome, rheumatoid arthritis, lupus erythematosus, and multiple sclerosis. The pathophysiology of ARG may be variable between patients including but not limited to accelerated metabolism of GABAnergic agents, defective binding of GABAnergic agents to GABAnergic receptors, defective function of G-proteins triggered by the binding of GABAnergic agents, defective triggering of messenger RNA production in response to GABAminergic binding, and defective protein translational activity in response to GABAnergic binding of receptors.

Without being bound by any particular theory, it is nonetheless apparent to the inventor that narcolepsy major and minor are due to defective monitoring of sleep debt in one or more centers located in or around the hypothalamus or defective sensitization of neurons to sleep debt. Therefore, although ARG and narcolepsy are very different diseases, it is apparent to the author that treatment of both ARG and narcolepsy is the same, namely treatment with one or more GABAnergic agents. As with narcolepsy, ARG may be treated with daytime stimulants or alerting agents such as caffeine, amphetamine, phentermine, methylphenidate, and modafanil.

For administration to non-human animals, the compositions may also be added to the animal feed or drinking water. It may be convenient to formulate the animal feed and drinking water compositions so that the animal takes in a therapeutically appropriate quantity of the composition along with its diet. It may also be convenient to present the composition as a premix for addition to the feed or drinking water.

### Means of Preventing Overdose

Treatment with multiple GABAnergic agents may result in overdose. Patients at risk for cardiac arrhythmia may have aggravation of arrhythmia based on the respiratory depressant effects of GABAminergic agents. In general, GHB; GBL; and 1,4-butanediol have greater respiratory depressant effects than Classes III-VI GABAminergic agents.

Furthermore, GABAnergic agents, especially GHB; GBL; and 1,4-butanediol may produce nausea and emesis in some patients, especially at high dosages. Thus, the patient could potentially vomit, and then in a state of stupor, intoxication, or sleepiness choke on vomitus, which can be fatal.

Class II GABAnergic agents available in dilute liquid solutions are the most easily titratable because the patient experiences progressive sleepiness as more beverage is consumed, and because relatively large volumes are required, she may stop consumption prior to overdose. The amount of GHB or alcoholic beverages required for individual patients is highly variable and even for the same patient the amount varies on a nightly basis, depending on the time of consumption, the rate of consumption, and whether food is co-ingested. If the solution or beverage is consumed over a long time period, greater dosages are required. Similarly, if food is ingested, greater dosages are required.

The doses of GABAnergic agents, especially GHB; GBL; and 1,4-butanediol, should be started low and titrated upwards slowly over weeks to months as tolerated by the patient and according to side effects such as headache, stupor, nausea, vomiting, enuresis, next day sedation, dysphoria, and coma. If the patient has nausea, she should stop taking the GABAnergic agents, such to avoid falling asleep and aspirating vomitus, which may be fatal.

If the patient is treated with a Class II agent such as NaGHB, the dose is typically started low at about 1.5 to 2 grams and slowly titrated upwards. Other GABAnergic agents are also added. When used as a lone agent, the sleep-inducing effects of GHB are typically only 2 to 3 hours, meaning that the patient inconveniently must take two to three doses per night. However, when used with other GABAnergic agents, GHB can often be given once prior to sleep.

The order in which GABAnergic agents are added to produce the most therapeutic regimen is variable; starting treatment with any GABAnergic agent prior to sleep or any combination of GABAnergic agents is encompassed in the invention. Sequentially adding any GABAnergic agent(s) to the regimen prior to sleep or about 2 to 8 hours after sleep onset is also encompassed in the invention.

In an aspect, the invention is a method of slowly increasing the dosages of GABAnergic agents to avoid toxicity. In an aspect, the invention is a method of treating patients with ARG, ARG type I and ARG type II comprising administering therapeutically effective amounts of two or more GABAnergic agents where each agent is independently selected from Class I GABAnergic agents, which are alcoholic beverages, Class II GABAnergic agents, in which Class II agents are gamma-hydroxyl butyric acid or pharmaceutically acceptable salts thereof; gamma-butyryl lactone; 1,4-butanediol; 4-HPA; 4-HPA lactone; Class III GABAnergic agents, which are benzodiazepines, Class IV GABAnergic agents, which bind subunits or configurations of the GABA receptor more specifically than benzodiazepines, Class V GABAnergic agents, which are structural analogs of gamma-amino butyric acid which do not bind the GABA receptor, and Class VI GABAnergic agents, which are barbiturates, to a patient in need of such treatment, such that the patient requires less frequency of dosing than the patient would otherwise require if GABAnergic agents were not combined. In a preferred aspect, the combination of GABAnergic agents is administered prior to sleep.

Failing to use adequate doses or variety of Class III-VI GABAnergic agents leads to the requirement of increased daily dosages of Class I or II agents for the treatment of ARG. Liver transaminase values and other liver function tests may be monitored periodically in patients who are treated with Class I agents.

The common pain and headache medication acetaminophen is known to be hepatotoxic. The hepatotoxicity is aggravated by alcoholic beverages. A single four gram dose of acetaminophen has been known to be fatal in chronic, heavy users of alcoholic beverages. The largest dose of acetaminophen commonly recommended to non-users of alcoholic beverages is one gram every four to six hours, not to exceed four grams in one day. Chronic users of alcoholic beverages for the treatment of ARG should be advised to limit the intake of acetaminophen. Chronic alcohol use is associated with hepatic cirrhosis in patients who do not have ARG. The risk of hepatic cirrhosis in patients who have ARG and consume large quantities of alcoholic beverages on a regular basis is unknown. Physicians who are concerned about hepatic cirrhosis due to Class I GABAnergic agents may prescribe other GABAnergic agents.

In an aspect, the invention comprises administering ribose in addition to one or more Class I-VI GABAnergic agents for the treatment of ARG. In a preferred aspect, ribose is administered at about 2 to 60 grams per day. In a preferred aspect ribose is administered as about 5 grams four times per day or 10 grams twice per day and one or more GABAnergic agents is given prior to sleep.

In an aspect, a patient is given a Class I GABAnergic agent about 2 to 12 hours prior to sleep onset to control the symptoms of ARG during waking hours. In a preferred aspect, the Class I GABAnergic agent is given about 6 hours prior to sleep onset. Alternatively, the patient is instructed to take the Class I GABAnergic agent allowing sufficient time for its effects to diminish or resolve prior to sleep. Prior to sleep, the patient is given one or more agents independently selected from Class II-VI GABAnergic agents in higher doses than would be required if the Class I agent were co-ingested with the Class II-VI agent, the higher doses of Class II-VI agents nonetheless decreasing the daily dose of Class I agents required. The preferred agents prior to sleep are Class III-V agents. In yet a more preferred aspect, the GABAnergic agent given prior to sleep in very high dose is a Class III agent. Preferred Class III agents when given in high doses have short to moderate duration of action to avoid prolonged sedation upon awakening. Alprazolam is a preferred Class III agent.

About 40 to 1500 ml red wine may be consumed about 6 hours prior to sleep. In an aspect, doses of other GABAnergic agents may be increased to about 4 to 10 mg alprazolam, 10 mg zolpidem, and 300 mg gabapentin may prior to sleep. Alternatively, about 4 to 8 mg clonazepam, 10 mg zolpidem, and 300 mg gabapentin may be taken prior to sleep.

In another aspect, a patient with ARG is given a Class I or II GABAnergic agent with one or more Class III-VI GABAnergic agents prior to sleep in which the Class I or II agent is used in a dose which is smaller than required if the Class III-VI agent(s) were not used, this dose being empirically determined for each patient and variable. The Class III-VI agent(s) are optimally chosen to have sufficient GABAnergic power to be therapeutic but to minimize side effects. The preferred agents are Class III-V agents.

A preferred Class III agent when used in high doses to decrease the amount of Class I or II agent required is alprazolam 2 to 10 mg prior to sleep. For the treatment of ARG, high doses of Class III agents such as alprazolam may be combined with Class IV and V agents, such as a regimen comprising about 7 mg alprazolam, 10 mg zolpidem, and 300 mg gabapentin. In an aspect, about 40 to 660 ml of red wine about 13.5% ethanol are consumed about 6 hours prior to sleep; furthermore about 7 mg alprazolam, 10 mg zolpidem, and 300 mg gabapentin are consumed just before sleep.

For the treatment of ARG, preferred dual agent combination therapies include a one or more Class III and one or more Class IV agents. The preferred Class III agents are alprazolam about 0.25-7 mg and clonazepam about 0.25-5 mg. The preferred Class IV agents are zolpidem about 2.5-60 mg and zaleplon about 2.5-60 mg. A Class III agent such as alprazolam or clonazepam may be combined with a Class V agent such as gabapentin or pregabalin. A Class IV agent such as zolpidem or zaleplon may be combined with may be combined with a Class V agent such as gabapentin or pregabalin. In a more preferred aspect, a dual agent combination is alprazolam about 2 to 7 mg and zolpidem about 2.5 to 50 mg. In a yet more preferred aspect, the dual agent combination is the Class III agent alprazolam about 2 to 5 mg and the Class IV agent zolpidem about 10 to 15 mg. These dual agent combinations may be combined with one or more Class I or II agents.

Preferred triple agent combination therapies include a one or more Class III and one or more Class IV agents and or one or more Class V agents. The preferred Class III agents are alprazolam about 0.25-7 mg and clonazepam about 0.25-5 mg. The preferred Class IV agents are zolpidem about 2.5-60 mg and zaleplon about 2.5-60 mg. The preferred Class V agents are gabapentin about 25 to 1000 mg and pregabalin about 25-1000 mg. In a more preferred aspect, the triple agent combination is alprazolam about 2 to 5 mg, zolpidem about 10 to 15 mg, and gabapentin about 300 to 500 mg. These triple agent combinations may be combined with one or more Class I or II agents.

### Means of Reducing Dose of Class I and II GABAnergic Agents

Although GHB; GBL; and 1,4-butanediol are generally safe medications, at high doses the medications can have side effects including nausea, vomiting, dysphoria, enuresis, headache, and respiratory depression. Chronic, heavy consumption of Class I GABAnergic agents is associated with hepatic cirrhosis and alcoholic pancreatitis in non-ARG patients and may be associated with hepatic cirrhosis and pancreatitis in ARG patients. Therefore, there is a need to treat ARG, insomnia, anxiety and other conditions listed herein with a method which decreases the consumption of Class I or Class II GABAnergic agents in patients who have unwanted side effects.

The dosage of Class I and II GABAnergic agents consumed prior to sleep, later in the sleep period, or during waking hours may be decreased by co-administering doses Class III, Class IV, Class V, and/or Class VI GABAnergic agents. The preferred agents are Class III, IV, and V agents. In an aspect, alprazolam is a preferred Class III agent. In an aspect, zolpidem is a preferred Class IV agent. In an aspect, gabapentin is a preferred Class V agent. In an aspect, the Class III, IV, V, and/or VI agents are co-administered with the Class II agent prior to sleep.

### Means of Reducing Dose of Class III GABAnergic Agents

Class III agents are associated with next day sedation. The dosage of Class III GABAnergic agents consumed prior to sleep, later in the sleep period, or during waking hours may be decreased by co-administering doses of Class I, Class II, Class IV, Class V, and/or Class VI GABAnergic agents. The preferred agents are Class I, II, IV, and V agents. In an aspect, GHB; GBL; 1,4-butanediol; 4-HPA; and 4-HPA lactone are preferred Class II agents. In an aspect, zolpidem is a preferred Class IV agent. In an aspect, gabapentin is a preferred Class V agent. In an aspect, the Class I, II, IV, V, and/or VI agents are co-administered with the Class III agent prior to sleep.

### Means of Vitamin Supplementation

Vitamins, such as the B-vitamins, are also useful in treating ARG or the side effects associated with taking one or more of the above identified GABAnergic agents. When treating ARG as indicated above, it is possible for the patient to develop a vitamin deficiency. For example, B12, folate, and thiamine deficiencies are associated with chronic, heavy use of Class I GABAnergic agents. If a cyancobalamin (vitamin B12) deficiency exists in the patient, it is typically treated by administering 1000 mcg of vitamin B12 by intramuscular (IM) injection once per week for four weeks, followed by a 1000 mcg injection (preferably IM) once per month.

Surprisingly, this is an inappropriately low dose for many patients regardless for the reason of the B12 deficiency. Low normal levels or even normal blood or serum levels of B12 can be associated with fatigue or neuropsychiatric symptoms. To counter these effects, B12 may be administered. When it is administered, dosages higher than those typically given may be used. For example, B12 may be administered as follows: about 1000 mcg IM every day to every other day for about 5 doses followed by about 500 to 2000 mcg IM injection about every 3 to 7 days.

In an aspect, a patient with ARG is treated with multiple GABAnergic agents in addition to B12. Subcutaneous or oral supplementation is also effective for some patients but less preferred. In an aspect, folate is replaced by about 5 to 10 mg orally on a daily basis. In another aspect, thiamine is replaced orally. A multivitamin may also be consumed on a daily basis.

As used herein, “GHB” refers to gamma hydroxyl butyric acid and non-toxic pharmaceutical salts thereof, “4-HPA” refers to 4-hydroxyl pentanoic acid and non-toxic pharmaceutical salts thereof, “4-HPA lactone” refers to 4-hydroxyl pentanoic acid lactone, and GBL refers to gamma-butyryl lactone.

Given the pathophysiologies of ARG and narcolepsy as described above, all of the following examples are expected to be therapeutic and useful for treating ARG, narcolepsy major, and narcolepsy minor.

The invention is further illustrated further by the following examples, which are not to be construed as limiting the invention in scope or spirit to the specific procedures described in them. The following examples, even where a specific disease or condition is described, can be used to treat a variety of diseases or conditions, as disclosed herein.

### EXAMPLE 1

The composition of the invention can be prepared by dissolving sodium gamma-hydroxybutyrate 4.235 grams into 150 ml water. 100 mg methyl paraben, 30 mg propyl paraben, a flavoring agent, and a coloring agent, and enough water to generate a total volume of 235 ml are then added. This affords a concentration of 18 mg of sodium gamma-hydroxybutyrate/ml of solution.

### EXAMPLE 2

The composition of the invention can be prepared by dissolving sodium gamma-hydroxybutyrate 4.235 grams into 150 ml water. 40 mg methyl paraben, 12 mg propyl paraben, a flavoring agent, and a coloring agent, and enough water to generate a total volume of 235 ml are then added. This affords a concentration of 18 mg of sodium gamma-hydroxybutyrate/ml of solution.

### EXAMPLE 3

The composition of the invention can be prepared using sodium gamma-hydroxybutyrate 4.235 grams, 168 mg methyl paraben, 50 mg propyl paraben, a flavoring agent, a coloring agent, and enough water to generate a total volume of 235 ml.

### EXAMPLE 4

The composition of the invention can be prepared using sodium gamma-hydroxybutyrate 4.235 grams, 100 mg methyl paraben, 30 mg propyl paraben, 0.2 grams sodium benzoate, a flavoring agent, and a coloring agent brought up to 235 ml with water.

### EXAMPLE 5

The composition of the invention can be prepared using sodium gamma-hydroxybutyrate 4.235 grams, 40 mg methyl paraben, 12 mg propyl paraben, 0.2 grams sodium benzoate, a flavoring agent, and a coloring agent brought up to 235 ml with water.

### EXAMPLE 6

The composition of the invention can be prepared by dissolving 3.025 grams of 1,4-butanediol into 150 ml water, 100 mg methyl paraben, 30 mg propyl paraben, a flavoring agent, and a coloring agent, and enough water to generate a total volume of 235 ml are then added. This affords a concentration of 12.9 mg of 1,4-butanediol/ml of solution.

### EXAMPLE 7

The composition of the invention can be prepared by dissolving 2.89 grams of GBL into 150 ml water. 100 mg methyl paraben, 30 mg propyl paraben, a flavoring agent, and a coloring agent, and enough water to generate a total volume of 235 ml are then added. This affords a concentration of 12.3 mg of GBL/ml of solution.

### EXAMPLE 8

The composition of the invention can be prepared using 3.025 grams 1,4-butanediol; 168 mg methyl paraben; 50 mg propyl paraben; a flavoring agent; a coloring agent; and enough water to generate a total volume of 235 ml.

### EXAMPLE 9

The composition of the invention can be prepared using 2.89 grams GBL, 168 mg methyl paraben, 50 mg propyl paraben, a flavoring agent, a coloring agent, and enough water to generate a total volume of 235 ml.

### EXAMPLE 10

The composition of the invention can be prepared using 3.025 grams 1,4-butanediol; 100 mg methyl paraben; 30 mg propyl paraben; 0.2 grams sodium benzoate; a flavoring agent; and a coloring agent brought up to 235 ml with water.

### EXAMPLE 11

The composition of the invention can be prepared using 2.89 grams GBL, 100 mg methyl paraben, 30 mg propyl paraben, 0.2 grams sodium benzoate, a flavoring agent, and a coloring agent brought up to 235 ml with water.

### EXAMPLE 12

The composition of the invention can be prepared adding in aqueous solution 3.025 grams 1,4-butanediol; 0.2 grams sodium benzoate; 7.05 grams citric acid; a flavoring agent; and a coloring agent brought up to 235 ml with water and adjusted to a pH of 3.2.

### EXAMPLE 13

The composition of the invention can be prepared adding in aqueous solution 3.025 grams 1,4-butanediol; 0.2 grams sodium benzoate; 1.9 grams citric acid; a flavoring agent; and a coloring agent brought up to 235 ml with water and adjusted to a pH of 3.2.

### EXAMPLE 14

The composition of the invention can be prepared adding in aqueous solution 3.025 grams 1,4-butanediol; 0.2 grams sodium benzoate; 1.9 grams citric acid; 0.4 grams trisodium citrate; a flavoring agent; and a coloring agent brought up to 235 ml with water and adjusted to a pH of 3.2.

### EXAMPLE 15

The composition is prepared adding in aqueous solution 2.89 grams GBL, 0.2 grams sodium benzoate, 7.05 grams citric acid, a flavoring agent, and a coloring agent brought up to 235 ml with water and adjusted to a pH of 3.2.

### EXAMPLE 16

The composition is prepared adding in aqueous solution 2.89 grams GBL, 0.2 grams sodium benzoate, 1.9 grams citric acid, a flavoring agent, and a coloring agent brought up to 235 ml with water and adjusted to a pH of 3.2.

### EXAMPLE 17

The composition is prepared adding in aqueous solution 2.89 grams GBL, 0.2 grams sodium benzoate, 1.9 grams citric acid, 0.4 grams trisodium citrate, a flavoring agent, and a coloring agent brought up to 235 ml with water and adjusted to a pH of 3.2.

### EXAMPLE 18

A concentrated solution of 1,4-butanediol is produced by adding in aqueous solution 200 grams 1,4-butanediol; 425.5 mg methyl paraben; 127.7 mg propyl paraben; a flavoring agent; and a coloring agent brought up to 1 liter with water.

### EXAMPLE 19

A concentrated solution of 1,4-butanediol is produced by adding in aqueous solution 200 grams 1,4-butanediol; 714.8 mg methyl paraben; 212.8 mg propyl paraben; a flavoring agent; and a coloring agent brought up to 1 liter with water.

### EXAMPLE 20

A concentrated solution of GBL is produced by adding in aqueous solution 200 grams GBL, 425.5 mg methyl paraben, 127.7 mg propyl paraben, a flavoring agent, and a coloring agent brought up to 1 liter with water.

### EXAMPLE 21

A concentrated solution of GBL is produced by adding in aqueous solution 200 grams GBL, 714.8 mg methyl paraben, 212.8 mg propyl paraben, a flavoring agent, and a coloring agent brought up to 1 liter with water.

### EXAMPLE 22

A concentrated solution of 1,4-butanediol is produced by mixing in aqueous solution 200 grams 1,4-butanediol; 1 gram sodium benzoate; 30 grams citric acid; a flavoring agent; and a coloring agent brought up to 1 liter with water and adjusted to a pH of 3.2.

### EXAMPLE 23

A concentrated solution of 1,4-butanediol is produced by mixing in aqueous solution 200 grams 1,4-butanediol; 1 gram sodium benzoate; 8 grams citric acid; a flavoring agent; and a coloring agent brought up to 1 liter with water and adjusted to a pH of 3.2.

### EXAMPLE 24

A concentrated solution of 1,4-butanediol is produced by mixing in aqueous solution 200 grams 1,4-butanediol; 1 gram sodium benzoate; 8 grams citric acid; 1.7 grams trisodium citrate; a flavoring agent; and a coloring agent brought up to 1 liter with water and adjusted to a pH of 3.2.

### EXAMPLE 25

A concentrated solution of GBL is produced adding in aqueous solution 200 grams GBL, 1 gram sodium benzoate, 30 grams citric acid, a flavoring agent, and a coloring agent brought up to 1 liter with water and adjusted to a pH of 3.2.

### EXAMPLE 26

A concentrated solution of GBL is produced adding in aqueous solution 200 grams GBL, 1 gram sodium benzoate, 8 grams citric acid, a flavoring agent, and a coloring agent brought up to 1 liter with water and adjusted to a pH of 3.2.

### EXAMPLE 27

A concentrated solution of GBL is produced adding in aqueous solution 200 grams GBL, 1 gram sodium benzoate, 8 grams citric acid, 1.7 grams trisodium citrate, a flavoring agent, and a coloring agent brought up to 1 liter with water and adjusted to a pH of 3.2.

### EXAMPLE 28

A pill or block may be produced by combining 2.5 grams of the sodium salt of gamma-hydroxyl butyric acid, 3 grams sodium carbonate, 3.6 grams citric acid, a flavoring agent, and a coloring agent. The pill or cake should be diluted in aqueous solution to maintain physiologically tolerable concentration of gamma-hydroxyl butyrate acid and pH. In an aspect, the pill or cake is diluted into about 45 to 500 ml aqueous solution.

### EXAMPLE 29

The composition of the invention can be prepared by dissolving 3.8 grams of the sodium salt of 4-hydroxyl pentanoic acid into 150 ml water, 100 mg methyl paraben, 30 mg propyl paraben, a flavoring agent, and a coloring agent, and enough water to generate a total volume of 235 ml are then added. This affords a concentration of 16.2 mg of the sodium salt of 4-hydroxyl pentanoic acid/ml of solution.

### EXAMPLE 30

The composition of the invention can be prepared by dissolving 2.7 grams of 4-hydroxyl pentanoic acid lactone into 150 ml water. 100 mg methyl paraben, 30 mg propyl paraben, a flavoring agent, and a coloring agent, and enough water to generate a total volume of 235 ml are then added. This affords a concentration of 11.5 mg of 4-hydroxyl pentanoic acid lactone/ml of solution.

### EXAMPLE 31

The composition of the invention can be prepared using 4.7 grams of the sodium salt of 4-hydroxyl pentanoic acid; 168 mg methyl paraben; 50 mg propyl paraben; a flavoring agent; a coloring agent; and enough water to generate a total volume of 235 ml.

### EXAMPLE 32

The composition of the invention can be prepared using 3.4 grams 4-hydroxyl pentanoic acid lactone, 168 mg methyl paraben, 50 mg propyl paraben, a flavoring agent, a coloring agent, and enough water to generate a total volume of 235 ml.

### EXAMPLE 33

The composition of the invention can be prepared using 4.7 grams of the sodium salt of 4-hydroxyl pentanoic acid; 100 mg methyl paraben; 30 mg propyl paraben; 0.2 grams sodium benzoate; a flavoring agent; and a coloring agent brought up to 235 ml with water.

### EXAMPLE 34

The composition of the invention can be prepared using 3.4 grams 4-hydroxyl pentanoic acid lactone, 100 mg methyl paraben, 30 mg propyl paraben, 0.2 grams sodium benzoate, a flavoring agent, and a coloring agent brought up to 235 ml with water.

### EXAMPLE 35

The composition of the invention can be prepared adding in aqueous solution 4.7 grams of the sodium salt of 4-hydroxyl pentanoic acid; 0.2 grams sodium benzoate; 7.05 grams citric acid; a flavoring agent; and a coloring agent brought up to 235 ml with water and adjusted to a pH of 3.2.

### EXAMPLE 36

The composition of the invention can be prepared adding in aqueous solution 4.7 grams of the sodium salt of 4-hydroxyl pentanoic acid; 0.2 grams sodium benzoate; 1.9 grams citric acid; a flavoring agent; and a coloring agent brought up to 235 ml with water and adjusted to a pH of 3.2.

### EXAMPLE 37

The composition of the invention can be prepared adding in aqueous solution 4.7 grams of the sodium salt of 4-hydroxyl pentanoic acid; 0.2 grams sodium benzoate; 1.9 grams citric acid; 0.4 grams trisodium citrate; a flavoring agent; and a coloring agent brought up to 235 ml with water and adjusted to a pH of 3.2.

### EXAMPLE 38

The composition is prepared adding in aqueous solution 3.4 grams 4-hydroxyl pentanoic acid lactone, 0.2 grams sodium benzoate, 7.05 grams citric acid, a flavoring agent, and a coloring agent brought up to 235 ml with water and adjusted to a pH of 3.2.

### EXAMPLE 39

The composition is prepared adding in aqueous solution 3.4 grams 4-hydroxyl pentanoic acid lactone, 0.2 grams sodium benzoate, 1.9 grams citric acid, a flavoring agent, and a coloring agent brought up to 235 ml with water and adjusted to a pH of 3.2.

### EXAMPLE 40

The composition is prepared adding in aqueous solution 3.4 grams 4-hydroxyl pentanoic acid lactone, 0.2 grams sodium benzoate, 1.9 grams citric acid, 0.4 grams trisodium citrate, a flavoring agent, and a coloring agent brought up to 235 ml with water and adjusted to a pH of 3.2.

### EXAMPLE 41

A concentrated solution of the sodium salt of 4-hydroxyl pentanoic acid is produced by adding in aqueous solution 311 grams of the sodium salt of 4-hydroxyl pentanoic acid; 425.5 mg methyl paraben; 127.7 mg propyl paraben; a flavoring agent; and a coloring agent brought up to 1 liter with water.

### EXAMPLE 42

A concentrated solution of the sodium salt of 4-hydroxyl pentanoic acid is produced by adding in aqueous solution 311 grams of the sodium salt of 4-hydroxy pentanoic acid; 714.8 mg methyl paraben; 212.8 mg propyl paraben; a flavoring agent; and a coloring agent brought up to 1 liter with water.

### EXAMPLE 43

A concentrated solution of 4-hydroxyl pentanoic acid lactone is produced by adding in aqueous solution 233 grams 4-hydroxyl pentanoic acid lactone, 425.5 mg methyl paraben, 127.7 mg propyl paraben, a flavoring agent, and a coloring agent brought up to 1 liter with water.

### EXAMPLE 44

A concentrated solution of 4-hydroxyl pentanoic acid lactone is produced by adding in aqueous solution 233 grams 4-hydroxyl pentanoic acid lactone, 714.8 mg methyl paraben, 212.8 mg propyl paraben, a flavoring agent, and a coloring agent brought up to 1 liter with water.

### EXAMPLE 45

A concentrated solution of the sodium salt of 4-hydroxyl pentanoic acid is produced by mixing in aqueous solution 311 grams of the sodium salt of 4-hydroxyl pentanoic acid; 1 gram sodium benzoate; 30 grams citric acid; a flavoring agent; and a coloring agent brought up to 1 liter with water and adjusted to a pH of 3.2.

### EXAMPLE 46

A concentrated solution of the sodium salt of 4-hydroxyl pentanoic acid lactone is produced by mixing in aqueous solution 311 grams of the sodium salt of 4-hydroxyl pentanoic acid; 1 gram sodium benzoate; 8 grams citric acid; a flavoring agent; and a coloring agent brought up to 1 liter with water and adjusted to a pH of 3.2.

### EXAMPLE 47

A concentrated solution of the sodium salt of 4-hydroxyl pentanoic acid is produced by mixing in aqueous solution 311 grams of the sodium salt of 4-hydroxyl pentanoic acid; 1 gram sodium benzoate; 8 grams citric acid; 1.7 grams trisodium citrate; a flavoring agent; and a coloring agent brought up to 1 liter with water and adjusted to a pH of 3.2.

### EXAMPLE 48

A concentrated solution of 4-hydroxyl pentanoic acid lactone is produced adding in aqueous solution 233 grams 4-hydroxyl pentanoic acid lactone, 1 gram sodium benzoate, 30 grams citric acid, a flavoring agent, and a coloring agent brought up to 1 liter with water and adjusted to a pH of 3.2.

### EXAMPLE 49

A concentrated solution of 4-hydroxyl pentanoic acid lactone is produced adding in aqueous solution 233 grams 4-hydroxyl pentanoic acid lactone, 1 gram sodium benzoate, 8 grams citric acid, a flavoring agent, and a coloring agent brought up to 1 liter with water and adjusted to a pH of 3.2.

### EXAMPLE 50

A concentrated solution of 4-hydroxyl pentanoic acid lactone is produced adding in aqueous solution 233 grams 4-hydroxyl pentanoic acid lactone, 1 gram sodium benzoate, 8 grams citric acid, 1.7 grams trisodium citrate, a flavoring agent, and a coloring agent brought up to 1 liter with water and adjusted to a pH of 3.2.

### EXAMPLE 51

A pill or block may be produced by combining 2.5 grams of the sodium salt of gamma-hydroxyl butyric acid, 3 grams sodium carbonate, 3.6 grams citric acid, a flavoring agent, and a coloring agent. The pill or cake should be diluted in aqueous solution to maintain physiologically tolerable concentration of gamma-hydroxyl butyrate acid and pH. In an aspect, the pill or cake is diluted into about 45 to 500 ml aqueous solution.

### EXAMPLE 52

A pill or block may be produced by combining 3.5 grams of the sodium salt of 4-hydroxyl pentanoic acid, 3 grams sodium carbonate, 3.6 grams citric acid, a flavoring agent, and a coloring agent. The pill or cake should be diluted in aqueous solution to maintain physiologically tolerable concentration of 4-hydroxyl pentanoate and pH. In an aspect, the pill or cake is diluted into about 45 to 500 ml aqueous solution.

### EXAMPLE 53

4-HPA was administered to two human subjects and 4-HPA lactone was administered to five human subjects. At equivalent doses of GHB, the patients would have experienced toxicity or sleep. However, the patients experienced for the most part relaxing effects. This shows that both 4-HPA and 4-HPA lactone are less toxic than GHB.

### EXAMPLE 54

A patient with ARG was chronically treated with the Class III GABAnergic agent alprazolam 3 mg, the Class IV agent zolpidem 10 mg, the Class V agent gabapentin 300 mg, and either the Class II agent NaGHB 5.25 grams or the Class I agent red wine about 1000 ml prior to sleep on a nightly basis and had marked reduction in fatigue and markedly increased motivation on these regimens. The patient was then switched to monotherapy using only NaGHB 4.5 grams prior to the intended sleep time and a second dose of 4.5 grams four hours after the first. The patient complained that the first dose did not induce sleep at all but did induce a state of near stupor until the second dose, and the second dose induced sleep for only two hours. There was no improvement on monotherapy with NaGHB. The patient was switched back to treatment with multiple GABAnergic agents and again had dramatic reduction in fatigue. On a scale from 0 to 10 where 10 is the most tired, the patient rated the fatigue at 10 without treatment, 3 to 4 with treatment on multiple GABAnergic agents, and 10 on monotherapy with NaGHB.

### EXAMPLE 56

A patient with ARG was treated with the Class IV agent zolpidem 10 mg, The Class III agent alprazolam 2 mg, the Class V agent gabapentin 200 mg, and the Class II agent sodium salt of gamma-hydroxyl butyrate 5.25 grams prior to sleep. The patient was treated chronically on a nightly basis. Sometimes, according to the patient's judgement, 3.75 grams of the sodium salt of gamma-hydroxyl butyric acid was consumed upon awakening, which usually was about six hours after the first dose of medications. The second dose of GHB usually did not induce sleep. The patient experienced markedly more restful sleep, markedly decreased fatigue, and increased motivation on this regimen.

### EXAMPLE 57

In one example a patient takes the Class IV agent zolpidem about 5 to 30 mg, the Class III agent alprazolam 0.25 to 4 mg or clonazepam 0.25 to 4 mg, the Class V agent gabapentin 100 to 800 mg, and the Class II agent the sodium salt of gamma-hydroxyl butyrate 2 to 5.25 grams prior to sleep. This regimen is useful for treating people with narcolepsy major or minor, acquired resistance to GABAnergic agents, and some patients with insomnia. Some patients require the maximum dosages. Patients with untreated sleep apnea may need dosage reductions. The dosages of medications should be started low and slowly increased over weeks to months. The patient should be maintained on the regimen for years or decades on a nightly or daily basis prior to sleep. The maximum typically recommended single dose of alprazolam and clonazepam is 2 mg. However, patients may be treated with doses higher than the typically recommended doses of the Class III agents such as alprazolam and clonazepam.

### EXAMPLE 58

In one example a patient takes the Class IV agent zaleplon about 5 to 60 mg, the Class III agent alprazolam 0.25 to 4 mg, the Class V agent gabapentin 100 to 800 mg, and the Class II agent the sodium salt of gamma-hydroxyl butyrate 2 to 5.25 grams prior to sleep. This regimen is useful for treating people with narcolepsy major or minor, acquired resistance to GABAnergic agents, and patients with insomnia. Some patients require the maximum dosages. Patients with untreated sleep apnea may need dosage reductions. The dosages of medications should be started low and slowly increased over weeks to months. The patient should be maintained on the regimen for years or decades on a nightly or daily basis prior to sleep.

### EXAMPLE 59a

In one example a patient takes the Class IV agent zolpidem about 5 to 30 mg, the Class III agent alprazolam 0.5 to 4 mg, the Class V agent gabapentin 100 to 800 mg, and the Class II agent gamma-butyryl lactone at a dose of about 1.4 to 3.6 grams This regimen is useful for treating people with narcolepsy major or minor, acquired resistance to GABAnergic agents, and some patients with insomnia. Patients with untreated sleep apnea may need dosage reductions. The dosages of medications should be started low and slowly increased over weeks to months.

### EXAMPLE 59b

In one example a patient takes the Class IV agent zaleplon about 5 to 60 mg, the Class III agent alprazolam about 0.5 to 4 mg, the Class V agent gabapentin 100 to 800 mg, and the Class II agent gamma-butyryl lactone about 1.4 to 4 grams. This regimen is useful for treating people with narcolepsy major or minor, acquired resistance to GABAnergic agents, and some patients with insomnia. Patients with untreated sleep apnea may need dosage reductions. The dosages of medications should be started low and slowly increased over weeks to months.

### EXAMPLE 59c

In one example a patient takes the Class IV agent zolpidem about 5 to 30 mg, the Class III agent alprazolam 0.5 to 4 mg, the Class V agent gabapentin 100 to 800 mg, and the Class II agent 1,4-butanediol about 1.4 to 3.7 grams. This regimen is useful for treating people with narcolepsy major or minor, acquired resistance to GABAnergic agents, and patients with insomnia. Patients with untreated sleep apnea may need dosage reductions. The dosages of medications should be started low and slowly increased over weeks to months.

### EXAMPLE 59d

In one example a patient takes the Class IV agent zaleplon about 5 to 60 mg, the Class III agent alprazolam 0.5 to 4 mg, the Class V agent gabapentin 100 to 800 mg, and the Class II agent 1,4-butanediol about 1.4 to 3.7 grams. This regimen is useful for treating people with narcolepsy major or minor, acquired resistance to GABAnergic agents, and patients with insomnia. Patients with untreated sleep apnea may need dosage reductions. The dosages of medications should be started low and slowly increased over weeks to months.

### EXAMPLE 59e

In one example a patient takes the Class IV agent zolpidem about 5 to 30 mg, the Class III agent alprazolam 0.25 to 4 mg, the Class V agent gabapentin 100 to 800 mg, the Class II agent 1,4-butanediol 0.4 to 1.2 grams, and the Class II agent gamma-butyryl lactone 0.4 to 1.2 grams. This regimen is useful for treating people with narcolepsy major or minor, acquired resistance to GABAminergic agents, and some patients with insomnia. Patients with untreated sleep apnea may need dosage reductions. The dosages of medications should be started low and slowly increased over weeks to months.

### EXAMPLE 59f

In one example a patient takes the Class IV agent zaleplon about 5 to 60 mg, the Class III agent alprazolam 0.25 to 4 mg, the Class V agent gabapentin 100 to 800 mg, the Class II agent 1,4-butanediol 0.4 to 1.2 grams, and the Class II agent gamma-butyryl lactone 0.4 to 1.2 grams. This regimen is useful for treating people with narcolepsy major or minor, acquired resistance to GABAminergic agents, and some patients with insomnia. Patients with untreated sleep apnea may need dosage reductions. The dosages of medications should be started low and slowly increased over weeks to months.

### EXAMPLE 59g

In one example a patient takes the Class IV agent zolpidem about 5 to 30 mg; the Class III agent alprazolam 0.25 to 4 mg; the Class V agent gabapentin 100 to 800 mg; the Class II agent 1,4-butanediol 0.7 to 3.6 grams; and the Class II agent the sodium salt of GHB 1 to 2.6 grams. This regimen is useful for treating people with narcolepsy major or minor, acquired resistance to GABAnergic agents, and some patients with insomnia. Patients with untreated sleep apnea may need dosage reductions. The dosages of medications should be started low and slowly increased over weeks to months. The patient should be maintained on the regimen for years or decades on a nightly basis.

### EXAMPLE 60

In one example a patient takes the Class IV agent zaleplon about 5 to 60 mg; the Class III agent alprazolam 0.25 to 4 mg; the Class V agent gabapentin 100 to 800 mg; the Class II agent 1,4-butanediol 0.7 to 3.6 grams; and the Class II agent the sodium salt of GHB 1 to 2.6 grams. This regimen is useful for treating people with narcolepsy major or minor, acquired resistance to GABAnergic agents, and some patients with insomnia. Patients with untreated sleep apnea may need dosage reductions. The dosages of medications should be started low and slowly increased over weeks to months. The patient should be maintained on the regimen for years or decades on a nightly basis.

### EXAMPLE 61

In one example a patient takes the Class II agent zolpidem about 5 to 30 mg, the Class III agent alprazolam 0.25 to 4 mg, the Class V agent gabapentin 100 to 800 mg, the Class II agent gamma-butyryl lactone 0.7 to 2.5 grams, and the Class II agent the sodium salt of GHB about 1 to 2.6 grams. This regimen is useful for treating people with narcolepsy major or minor, acquired resistance to GABAnergic agents, and some patients with insomnia. Patients with untreated sleep apnea may need dosage reductions. The dosages of medications should be started low and slowly increased over weeks to months.

### EXAMPLE 62

In one example a patient takes the Class IV agent zolpidem about 5 to 30 mg, the Class III agent alprazolam 0.25 to 4 mg, the Class V agent gabapentin 100 to 800 mg, the Class II agent gamma-butyryl lactone 0.7 to 2.5 grams, and the Class II agent the sodium salt of GHB about 1 to 2.6 grams. This regimen is useful for treating people with narcolepsy major or minor, acquired resistance to GABAnergic agents, and some patients with insomnia. Patients with untreated sleep apnea may need dosage reductions. The dosages of medications should be started low and slowly increased over weeks to months.

### EXAMPLE 63

In another example a patient with ARG was given the Class I agent 660 ml red wine 13.5% alcohol by volume, the Class III agent alprazolam 3 mg, the Class V agent gabapentin 300 mg, and the Class IV agent 10 mg zolpidem. Five hours later, the patient was awoken and given the Class II agent the sodium salt of gamma-hydroxyl butyric acid 3 grams. The patient slept an additional three hours and had dramatic decrease in fatigue.

### EXAMPLE 64

In another example a patient with ARG was treated with the Class I agent 660 ml red wine 13.5% alcohol by volume, the Class I agent liqeur 30% alcohol by volume 200 ml, the Class III agent alprazolam 2 mg, the Class IV agent gabapentin 200 mg, and the Class II agent the sodium salt of gamma-hydroxyl butyric acid 4 grams prior to sleep. The patient experienced markedly more restful sleep, markedly decreased fatigue, increased motivation, and had little to no side effects.

It should be noted that a proportion of the population would experience toxicity, aspiration, vomiting, or death on such large doses of GABAnergic agents, especially the combination of red wine and Class II agents given to this patient. However, the treating physician had slowly titrated the doses of GABAnergic agents in this patient and was comfortable treating the patient with large doses.

### EXAMPLE 65

In another example a patient with ARG is treated with the Class II agent the sodium salt of gamma-hydroxyl butyric acid 2 to 5.25 grams combined with the Class IV agent zolpidem 5 to 20 mg prior to sleep. Some patients may require the maximum dosages of both medications.

### EXAMPLE 66

In another example a patient with ARG is treated with the Class II agent the sodium salt of gamma-hydroxyl butyric acid 2 to 5.25 grams combined with the Class IV agent 5 to 60 mg of zaleplon prior to sleep.

### EXAMPLE 67

In another example, a patient with ARG is treated with the Class II agent the sodium salt of gamma-hydroxyl butyric acid 2 to 5.25 grams combined with the Class III agent about 0.25 to 3 mg of alprazolam prior to sleep.

### EXAMPLE 68

In another example, a patient with ARG is treated with the Class I agent red wine about 400 ml and about 13% alcohol by volume, the Class II agent the sodium salt of gamma-hydroxyl butyric acid 2 grams, the Class IV agent zolpidem 10 mg, The Class III agent alprazolam 3 mg, and the Class V agent gabapentin 500 mg prior to sleep. Doses should be started low and slowly increased as tolerated.

### EXAMPLE 69

In another example, a patient with ARG is treated with the Class I agent red wine about 400 ml and about 13% alcohol by volume, the Class II agent the sodium salt of gamma-hydroxyl butyric acid 2 grams, the Class IV agent zaleplon 20 mg, the Class III agent alprazolam 3 mg, and the Class V agent gabapentin 200 mg prior to sleep. Doses should be started low and slowly increased as tolerated.

### EXAMPLE 70

In another example, a patient with ARG is treated with the Class I agent wine 700 ml consumed over about 30 minutes to six hours prior to sleep, then the patient takes the Class II agent NaGHB 3 grams, the Class III agent alprazolam 3 mg, and the Class V agent gabapentin 300 mg. Upon awakening or 2 to 8 hours later awoken by alarm clock or spontaneously, the patient is given the Class I agent wine about 200 ml with a Class IV agent zolipidem 10 mg or zaleplon 20 mg to obtain further sleep. Such a regimen is useful for patients with ARG, RLS and/or PLMD, insomnia, and patients with ARG and narcolepsy major or narcolepsy minor. Some patients require lesser or greater dosages or lesser or greater varieties of GABAnergic agents in their regimens. Such regimens should be started at low doses and increased over time to avoid overdosage, coma, respiratory depression, and aspiration of vomitus, which may be fatal.

### EXAMPLE 71

In another example, a patient with ARG may be treated with the Class II agent NaGHB about 2 to 4 grams in addition to the Class VI agent phenobarbital 50 mg prior to sleep.

### EXAMPLE 72

In another example, a patient with ARG is treated with the Class I agent red wine about 200 to 1500 ml about 13% alcohol by volume in addition to the Class II agent NaGHB about 3 grams prior to sleep. Roughly 2 to 8 hours after sleep onset, whether the patient awakens spontaneously or by alarm, the patient is given a second dose of the Class II agent NaGHB or the Class I agent red wine. Many other combinations for the second dose are possible such as the Class IV agent zolpidem 10 mg with the Class III agent alprazolam 2 mg. Such regimens should be started at low doses and increased over time to avoid overdosage, coma, respiratory depression, and aspiration of vomitus, which may be fatal.

### EXAMPLE 73

A patient with acquired resistance to GABAnergic agents, insomnia, refractory restless legs syndrome, or narcolepsy is treated with the Class II agent 1,4-butanediol about 1.4 to 3.75 grams in addition to the Class III agent alprazolam about 0.25 to 4 mg prior to sleep. Doses should be started and increased slowly over weeks to months.

### EXAMPLE 74

A patient with acquired resistance to GABAnergic agents, insomnia, refractory restless legs syndrome, or narcolepsy is treated with the Class II agent 1,4-butanediol about 1.4 to 3.75 grams in addition to the Class IV agent zolpidem about 5 to 30 mg prior to sleep. Doses should be started and increased slowly over weeks to months.

### EXAMPLE 75

A patient with acquired resistance to GABAnergic agents, insomnia, refractory restless legs syndrome, or narcolepsy is treated with the Class II agent 1,4-butanediol about 1.4 to 3.75 grams in addition to the Class IV agent zaleplon about 5 to 60 mg prior to sleep. Doses should be started and increased slowly over weeks to months.

### EXAMPLE 76

A patient with acquired resistance to GABAnergic agents, insomnia, refractory restless legs syndrome, or narcolepsy major or minor is treated with the Class II agent GBL about 1.4 to 3.6 grams in addition to the Class IV agent alprazolam about 0.5 to 4 mg prior to sleep.

### EXAMPLE 76a

A patient with acquired resistance to GABAnergic agents, insomnia, refractory restless legs syndrome, or narcolepsy major or minor is treated with the Class II agent GBL about 1.4 to 3.6 grams in addition to the Class IV agent zolpidem about 5 to 30 mg prior to sleep.

### EXAMPLE 76b

A patient with acquired resistance to GABAnergic agents, insomnia, refractory restless legs syndrome, or narcolepsy major or minor is treated with the Class II agent GBL about 1.4 to 3.6 grams in addition to the Class IV agent zaleplon about 5 to 60 mg prior to sleep.

### EXAMPLE 76c

A patient with acquired resistance to GABAnergic agents, insomnia, refractory restless legs syndrome, or narcolepsy major or minor is treated with the Class II agent 1,4-butanediol about 0.7 to 1.9 grams in addition to the Class II agent GBL about 0.7 to 1.8 grams in addition to the Class III agent alprazolam about 0.25 to 4 mg prior to sleep.

### EXAMPLE 76d

A patient with acquired resistance to GABAnergic agents, insomnia, refractory restless legs syndrome, or narcolepsy major or minor is treated with the Class II agent 1,4-butanediol 0.7 to 1.9 grams in addition to the Class II agent GBL about 0.7 to 1.8 grams in addition to the Class IV agent zolpidem about 5 to 30 mg prior to sleep.

### EXAMPLE 76e

A patient with acquired resistance to GABAnergic agents, insomnia, refractory restless legs syndrome, or narcolepsy major or minor is treated with the Class II agent 1,4-butanediol about 0.7 to 1.9 grams in addition to the Class II agent GBL about 0.7 to 1.8 grams in addition to the Class IV agent zaleplon about 5 to 60 mg prior to sleep.

### EXAMPLE 76f

A patient with acquired resistance to GABAnergic agents, insomnia, refractory restless legs syndrome, or narcolepsy major or minor is treated with the Class II agent the sodium salt of gamma-hydroxyl butyric acid about 1 to 2.7 grams in addition to the Class II agent GBL about 0.7 to 1.8 grams in addition to the Class III agent alprazolam about 0.25 to 4 mg prior to sleep.

### EXAMPLE 76g

A patient with acquired resistance to GABAnergic agents, insomnia, refractory restless legs syndrome, or narcolepsy major or minor is treated with the Class II agent the sodium salt of gamma-hydroxyl butyric acid about 1 to 2.7 grams in addition to the Class II agent GBL about 0.7 to 1.8 grains in addition to the Class IV agent zolpidem about 5 to 30 mg prior to sleep.

### EXAMPLE 76h

A patient with acquired resistance to GABAnergic agents, insomnia, refractory restless legs syndrome, or narcolepsy major or minor is treated with the Class II agent the sodium salt of gamma-hydroxyl butyric acid about 1 to 2.7 grams in addition to the Class II agent GBL about 0.7 to 1.8 grams in addition to the Class IV zaleplon prior about 5 to 60 mg prior to sleep.

### EXAMPLE 76i

A patient with acquired resistance to GABAnergic agents, insomnia, refractory restless legs syndrome, or narcolepsy major or minor is treated with the Class II agent the sodium salt of gamma-hydroxyl butyric acid about 1 to 2.7 grams in addiction to the Class II agent GBL about 0.7 to 1.9 grams in addition to the Class III agent alprazolam about 0.25 to 4 mg prior to sleep.

### EXAMPLE 77j

A patient with acquired resistance to GABAnergic agents, insomnia, refractory restless legs syndrome, or narcolepsy major or minor is treated with the Class II agent the sodium salt of gamma-hydroxyl butyric acid about 1 to 2.7 grams in addition to the Class II agent GBL about 0.7 to 1.9 grams in addition to the Class IV agent zolpidem about 5 to 30 mg prior to sleep.

### EXAMPLE 77k

A patient with acquired resistance to GABAnergic agents, insomnia, refractory restless legs syndrome, or narcolepsy major or minor is treated with the Class II agent the sodium salt of gamma-hydroxyl butyric acid about 1 to 2.7 grams in addition to the Class II agent GBL about 0.7 to 1.9 grams in addition to the Class IV agent zaleplon about 5 to 60 mg prior to sleep prior to sleep.

### EXAMPLE 78

A patient with ARG is given the Class II agent GBL about 1 to 5 grams prior to sleep and a repeat dose about two to six hours after sleep onset. The second dose may be given about 3 hours after sleep onset or upon spontaneously awakening.

### EXAMPLE 79

A patient with ARG is given the Class II agent 1,4-butanediol about 1 to 5 grams prior to sleep and a repeat dose about two to six hours after sleep onset. The second dose may be given about 3 hours after sleep onset or upon spontaneously awakening.

### EXAMPLE 80

Most patients with ARG require moderate to high doses of one or more GABAnergic agents. However, in another example a patient is treated with low doses of multiple GABAnergic agents, such as the Class II agent NaGHB 0.1 to 0.5 grams, the Class IV agent zolpidem 1 to 5 mg, the Class III agent alprazolam 0.1 to 0.5 mg, and/or the Class V agent gabapentin 25 to 100 mg.

### EXAMPLE 81

A patient who has side effects on a given dose of Class II GABAnergic agent is treated with a lower dose of the Class II agent and increased dosages of other GABAnergic agent(s). The dosage of GABAnergic agents tolerated by patients for Class II-VI agents is variable. A patient who has side effects due to the Class II agent GHB on 4 grams of NaGHB and the Class III agent alprazolam 2 mg may instead be treated with a lesser dose of NaGHB such as 2 grams and the alprazolam may be increased in dose and/or other GABAnergic agents such as the Class IV agent zolpidem or the Class V agent gabapentin may be added to the regimen. Given the wide variety of GABAnergic agents available, numerous other regimens are possible to decrease the dose of Class II agents.

### EXAMPLE 82

A patient with acquired resistance to GABAnergic agents was treated with the Class III agent alprazolam 2 mg, the Class IV agent zolpidem 10 mg, the Class V agent gabapentin 200 mg, and the Class II agent NaGHB 7 grams. The patient had markedly decreased fatigue and little to no toxicity. 7 grams NaGHB as a single dose is a large dose, which may be too toxic for some patients.

### EXAMPLE 83

A patient with acquired resistance to GABAnergic agents was treated with the Class I agent red wine 660 ml 13.5% alcohol by volume, then the Class III agent alprazolam 3 mg, the Class V agent gabapentin 300 mg, the Class IV agent zolpidem 10 mg, and the Class II agent NaGHB 9 grams. 4 hours after sleep onset the patient awoke in a neighboring room in a pool of vomitus of partially digested wine. The patient was lucid and had mild nausea. The patient went back to sleep in bed for an additional 2 hours and was able to work the next day. The patient had markedly decreased fatigue despite the interruption of sleep by vomiting. The combination of red wine and such large doses of GHB are not typically recommended by the inventor and can be fatal.

### EXAMPLE 84

A patient with acquired resistance to GABAminergic agents, insomnia, refractory restless legs syndrome, or narcolepsy is treated with the Class II agent 1,4-butanediol about 1.4 to 3.75 grams in addition to the Class III agent alprazolam 0.5 to 3 mg prior to sleep. Doses should be started and increased slowly over weeks to months.

### EXAMPLE 85

A patient with ARG or insomnia is treated with the Class I agent red wine 40 to 1500 ml about 13% alcohol by volume, the Class III agent alprazolam about 2 mg, the Class IV agent zolpidem 10 mg, the Class V agent gabapentin 300 mg, and the Class II agent the sodium salt of gamma-hydroxyl butyric acid about 2 to 5 grams. Doses should be started low and increased over weeks to months.

### EXAMPLE 86

A patient had a sleeping disorder. Overnight polysomnography had shown minimal obstructive sleep apnea with 2 apneas per hour of sleep. The patient was placed on a regimen of the Class III agent alprazolam, the Class IV agent zolpidem, the Class V agent gabapentin, and the Class II agent the sodium salt of gamma-hydroxyl butyric acid (GHB), which were administered prior to sleep for the treatment of sleeping disorders. The initial dose of GHB was prescribed as 2.25 grams prior to sleep and repeat dose of 2.25 grams to be taken 2.5 to 4 hours after sleep onset. Doses were slowly increased to a regimen of 3 mg alprazolam, 10 mg zolpidem, 200 mg gabapentin, and 5.25 grams NaGHB prior to sleep. However, the patient found that he slept roughly six hours when given the alprazolam, zolpidem, gabapentin, and 3 grams of NaGHB, and he instead took the second GHB dose upon awakening, not 2.5 to 4 hours after sleep onset as prescribed. The patient eventually increased the first dose of NaGHB to 5.25 grams prior to sleep, and sometimes took a second dose of 3.75 grams upon awakening. The second dose usually did not induce sleep.

Within one week of beginning to take the GHB, the patient had marked decrease in appetite. At a dose of 3 grams prior to sleep and a second dose upon awakening, the patient was eating only one to two small meals per day. His maximum previous weight had been 169 lbs and his baseline weight was 159 pounds. He lost weight down to 145 lbs and looked gaunt. He believed that the GHB had suppressed his appetite and consulted an endocrinologist, who did a thorough medical evaluation including an am cortisol, a comprehensive chemistry profile, a CBC, and thyroid studies, all of which were normal. The patient discontinued the GHB and his appetite returned within two days. Over roughly five months, he then gained weight up to 195 lbs and his weight remained stable.

Although GHB; GBL; 1,4-butanediol; 4-HPA; and 4-EPA lactone are not associated with anorexia (loss of appetite), they are useful in the treatment of obesity. The substances or combinations of the substances may be administered as a single dose prior to sleep onset or as multiple doses through the night. The substances may be administered prior to sleep in addition to other hypnotics or agents useful in the treatment of sleep disorders. Care should be taken not to aggravate obstructive or central sleep apnea by the administration of the medication. The substances may be administered during the day.

GHB; GBL; 1,4-butanediol; 4-HPA; and/or 4-HPA lactone may be administered for sleep while co-therapies for obesity may be administered during waking hours. Co-therapies include agents such as phentermine, phendimetrazine, benzphetamine, sibutramine, diethylpropion, orlistat, pseudoephedrine, fenfluramine, dexfenfluramine, amphetamine, amphetamine salts, dextroamphetamine, methylphenidate, ephedrine, ephedra, and pseudoephedrine, caffeine, and thyroid hormone.

### EXAMPLE 87

A patient with ARG was given the Class III agent alprazolam 7 mg, the Class IV agent zolpidem 10 mg, and the Class V agent gabapentin 300 mg prior to sleep. The patient had next day sedation due to the high dose of alprazolam. However, after the effects of the alprazolam had dissipated, the patient had markedly decreased fatigue. Such high doses of alprazolam are acceptable but often lead to next day sedation. The dose of alprazolam may be reduced by adding a Class I or Class II agent to the regimen.

### EXAMPLE 88

Most patients with ARG require a strong GABAnergic agent such as a Class I agent or a Class II agent in combination with other GABAnergic agents. However, in another example a patient with ARG is given the Class IV agent zolpidem 5 to 30 mg or zaleplon 5 to 60, the Class III agent alprazolam 0.25 to 4 mg, and the Class V agent gabapentin 100 to 800 mg prior to sleep. The preferred doses are about 10 mg zolpidem, 2 to 3 mg alprazolam, and 100 to 500 mg gabapentin. She may then be given additional GABAnergic agents such as zolpidem, zaleplon, or alprazolam, or gabapentin upon spontaneously awaking, either spontaneously or by alarm, to obtain further stabilized sleep.

### EXAMPLE 89

A patient with ARG who had minimal obstructive sleep apnea was chronically treated with the Class I agent red wine about 200 to 1500 ml 13.5% alcohol by volume, the Class IV agent zolpidem 10 mg, the Class III agent alprazolam 3 mg, and the Class V agent gabapentin 300 mg prior to sleep. Sufficient wine was consumed to produce moderate to strong sleepiness and then the other GABAminergic medications were consumed. When the volume of wine on a particular night required to induce strong sleepiness was inconvenient, the patient quickly drank liqeur 30% alcohol by volume, typically about 160 ml, in order to induce strong sleepiness. The patient experienced markedly more restful sleep, markedly decreased fatigue, and increased motivation on this regimen.

### EXAMPLE 90

A patient with ARG who had minimal obstructive sleep apnea was chronically treated with the Class I agent red wine about 660 to 1500 ml about 13% alcohol by volume, the Class IV agent zolpidem 10 mg, the Class III agent alprazolam 3 mg, and the Class V agent gabapentin 300 mg prior to sleep. Sufficient wine was consumed to produce moderate to strong sleepiness and then the other GABAnergic medications were consumed. The patient averaged about 1000 ml red wine per evening and night. The patient experienced markedly more restful sleep, markedly decreased fatigue, and increased motivation on this regimen.

### EXAMPLE 90a

A patient with ARG was chronically treated with the Class I GABAnergic agent wine about 1000 ml 13.5% alcohol by volume per evening in addition to the Class III agent aplrazolam 3 mg, the Class IV agent zolpidem 10 mg, and the Class V agent gabapentin 300 mg. The patient developed mouth sores. The patient was initially treated with the following regimen: one injection of cyanocobalamin 500 mcg IM, then rotate 5 mg orally every day to every day, and a mutivitamin containing thiamine 1.5 mg orally on a daily basis. The mouth sores resolved. Subsequently, the patient's cyanocobalamin dose was increased to 2000 mcg IM about every three days. The patient had a mild decrease in fatigue on the increased dose of cyanocobalamin in addition to the dramatic decrease in fatigue obtained by treating the patient with multiple GABAnergic agents.

### EXAMPLE 91

In another example a patient with ARG who has minimal obstructive sleep apnea is treated chronically with the Class I agent red wine 50 to 1500 ml about 13% alcohol by volume, the Class IV agent zolpidem 10 mg or zaleplon 20 mg, the Class III agent alprazolam 3 mg, and the Class V agent gabapentin 300 mg. Generally, sufficient wine is consumed to produce mild to strong sleepiness or desire to sleep and then the other GABAminergic medications are consumed. In order to titrate the wine to the correct volume, which may be variable from night to night, however, consumption of wine to a degree of sleepiness is useful. Some patients may require a second dose GABAnergic agent later in the night, such as the Class I agent red wine 50 to 700 ml sometimes with 40 ml vodka, if the red wine is about 13% alcohol by volume, and if the vodka is about 40% alcohol by volume, about 2 to 8 hours after sleep onset, or the Class IV agent 5 to 30 mg zolpidem about 2 to 8 hours after sleep onset.

### EXAMPLE 92

In another example a patient with ARG was treated with the Class I agent red wine 660 ml 13.5% alcohol by volume, the Class III agent alprazolam 3 mg, and the Class IV agent zolpidem 10 mg prior to sleep. The patient experienced markedly more restful sleep, markedly decreased fatigue, increased motivation, and had little to no side effects.

### EXAMPLE 93

In another example, a patient with ARG is treated with the Class I agent red wine about 210 ml if the wine is about 13% alcohol by volume in addition to sufficient liquor to produce mild to strong sleepiness or desire to sleep, such as 40 to 200 ml rum or liqueur, if the rum or liqueur is about 40% alcohol by volume, and then the patient takes the Class III agent alprazolam 0.5 to 3 mg in addition to the Class V agent 100 to 800 mg gabapentin prior to sleep. The class IV agent zolpidem may be substituted for the Class III agent alprazolam.

### EXAMPLE 94

In another example, a patient with ARG is treated with the Class I agents red wine about 50 to 1000 ml if the wine is about 13% alcohol by volume in addition to sufficient liquor to produce mild to strong sleepiness or desire to sleep, such as 40 to 200 ml rum, if the rum is about 40% alcohol by volume, and then the patient takes the Class IV agent zaleplon 5 to 60 mg prior to sleep.

### EXAMPLE 95

In another example, a patient with ARG is treated with the Class I agents red wine about 50 to 1500 ml if the wine is about 13% alcohol by volume prior to sleep in addition to sufficient liquor to produce mild to strong sleepiness or desire to sleep, such as 20 to 100 ml rum, if the rum is about 75% alcohol by volume, and then the patient takes a Class IV agent zolpidem 5 to 30 mg or zaleplon 5 to 60 mg.

### EXAMPLE 96

In another example, a patient with ARG was treated with the Class I agent about red wine about 750 ml 13.5% alcohol by volume, sometimes with additional liquor to induce sleepiness, the Class III agent alprazolam 3 mg, the Class V agent gabapentin 300 mg, and the Class IV agent zolpidem 10 mg prior to sleep on a nightly basis. The patient experienced markedly more restful sleep, markedly decreased fatigue, and increased motivation on this regimen.

### EXAMPLE 97

In another example, a patient with ARG was treated with the Class I agent red wine about 750 ml 13.5% alcohol by volume, sometimes with additional liquor to induce sleepiness, the Class III agent alprazolam 3 mg, and the Class V agent gabapentin 300 mg prior to sleep. The patient experienced markedly more restful sleep, markedly decreased fatigue, and increased motivation on this regimen.

### EXAMPLE 98

In another example, a patient with ARG is treated with the Class I agent red wine 750 ml, the Class III agent alprazolam 2 mg, the Class IV agent zolpidem 10 mg, and the Class V agent gabapentin 200 mg prior to sleep. About two to eight hours after sleep onset, if the patient has awoken spontaneously or by alarm clock, she then consumes the Class I agent red wine about 120 ml combined with a Class IV agent zolpidem 10 mg or zaleplon 20 mg to obtain further stabilized sleep.

### EXAMPLE 99

In another example, a patient with ARG was treated with the Class I agent red wine about 750 ml 13.5% alcohol by volume, the Class III agent alprazolam 2 mg, the Class IV agent zolpidem 10 mg, and the Class V agent gabapentin 200 mg prior to sleep. After awakening spontaneously, typically about six hours later, the patient then consumed about 120 ml red wine and 40 ml liqeur 30% alcohol by volume to obtain further stabilized sleep. The patient experienced markedly more restful sleep, markedly decreased fatigue, and increased motivation on this regimen.

### EXAMPLE 100

In another example, a patient with ARG was treated with the Class I agent red wine about 750 ml about six hours prior to the natural time of sleep onset. Upon subjective diminishment of the effects of the wine, the patient was treated with the Class III agent alprazolam 3 mg, the Class IV agent zolpidem 10 mg, and the Class V agent gabapentin 300 mg prior to sleep. The patient experienced markedly decreased fatigue and increased motivation on this regimen.

### EXAMPLE 101

In another example, a patient with ARG is treated about two to ten hours prior to the patient's natural time of sleep with the Class I agent red wine about 50 to 1500 ml. Liquor may be added to the wine to decrease the volume of Class I GABAnergic agents. Upon subjective diminishment of the effects of the wine, the patient is treated with the Class III agent alprazolam about 0.25 to 4 mg, the Class IV agent zolpidem 5 to 30 mg or zaleplon 5 to 60 mg, and the Class V agent gabapentin 100 to 800 mg prior to sleep.

### EXAMPLE 102

In another example, a patient with ARG is treated with the Class I agent liquor about 40 to 350 ml about 30% alcohol by volume about two to ten hours prior to her natural time of sleep onset. Upon subjective diminishment of the effects of the liquor, the patient is treated with the Class III agent alprazolam about 0.5 to 4 mg, a Class IV agent zolpidem about 5 to 30 mg or zaleplon 5 to 60 mg, and the Class V agent gabapentin about 100 to 800 mg prior to sleep. The Class IV agents zolpidem and zaleplon may be combined.

### EXAMPLE 103

In another example, a patient with ARG is treated with the Class I agent liquor about 40 to 350 ml about 30% alcohol by volume and/or about 250 to 750 ml red wine about two to ten hours prior to her natural time of sleep onset. Upon subjective diminishment of the effects of the Class I agents, the patient may is treated with about 0.5 to 4 mg alprazolam.

### EXAMPLE 104

In another example, a patient with ARG is treated with the Class I agent liquor about 30 to 350 ml about 30% alcohol by volume or about 250 to 750 ml red wine about two to fourteen hours prior to her natural time of sleep onset. Upon subjective diminishment of the effects of the Class I agent, the patient is treated with a Class IV agent zolpidem about 5 to 30 mg and/or zaleplon 5 to 60 mg.

### EXAMPLE 105

In another example, the patient with ARG consumes the Class I GABAnergic red wine 50 to 1000 ml combined with a Class IV agent zolpidem 5 to 30 mg or zaleplon 5 to 60 mg prior to sleep.

### EXAMPLE 106

In another example, a patient with ARG consumes Class I agents red wine 250 to 1000 ml, if the wine is about 13% alcohol by volume, in addition to rum about 40 to 80 ml, if the rum is about 35% alcohol by volume, combined with a Class IV agent zolpidem 5 to 30 mg or zaleplon 5 to 60 mg prior to sleep. Zolpidem may be combined with zaleplon.

### EXAMPLE 107

In another example, a patient with ARG consumes the Class I GABAnergic agent red wine 250 to 750 ml combined with the Class III agent about 0.25 to 4 mg alprazolam prior to sleep.

### EXAMPLE 108

In another example, a patient with ARG consumes the Class I agent red wine 250 to 1000 ml over about ten minutes to three hours, and at a point of mild to strong desire to sleep, then consumes the Class III agent alprazolam 3 mg, the Class V agent gabapentin 200 mg, a Class IV agent zolpidem 10 mg or zaleplon 20 mg, and then two to eight hours later is woken by alarm or spontaneously and consumes an additional 210 ml red wine and 40 ml rum, if the rum is about 40% alcohol by volume. The additional dosage of GABAnergic agents at the end of the sleep period is necessary to control ARG at the end of the sleep period. Suboptimal dosages of GABAnergic agents prior to sleep would require the use of increased dosages of GABAnergic agents, especially Class I agents, during waking hours. GABAnergic agents during waking hours provide partial relief of fatigue regardless of whether they induce sleep. The preferred GABAnegic agents during waking hours are Class I agents.

### EXAMPLE 109

In another example, a patient with ARG was treated with the Class I agent red wine 660 ml, the Class VI agent phenobarbital 100 mg, and the Class V agent gabapentin 300 mg prior to sleep. Sleep was found to be moderately more restful and the patient had decreased fatigue.

### EXAMPLE 110

In order to reduce the amount of Class I GABAnergic agent ingested, a patient with ARG was treated with the Class I agent red wine 420 ml 13.5% alcohol eight hours prior to sleep to control symptoms during waking hours and was treated with a larger dose of Class III GABAnergic agents prior to sleep, which was 7 mg alprazolam. The Class IV agent zolpidem 10 mg and the Class V agent gabapentin 300 mg were consumed with the alprazolam. The patient had markedly decreased fatigue and increased motivation on this regimen.

### EXAMPLE 111

Most patients with ARG require moderate to high doses of one or more GABAnergic agents. However, on occasion a patient may be treated with low doses of multiple GABAnergic agents, such as the Class IV agent zolpidem 1 to 5 mg, the Class III agent alprazolam 0.1 to 0.5 mg, and/or the Class V agent gabapentin 25 to 100 mg.

### EXAMPLE 112

A patient with ARG is treated with the Class IV agents about 1 gram GHB; 1 gram GBL; 1 gram 1,4-butanediol; 1 gram 4-HPA; and 1 gram 4-HPA lactone prior to sleep. About 2 to 8 hours after sleep onset, the patient is given a second dose of a Class II GABAnergic agent such as GHB; GBL; 1,4-butanediol; 4-HPA; 4-HPA lactone, or combinations thereof.

### EXAMPLE 113

A patient with ARG takes the Class II agents the sodium salt of gamma-hydroxyl butyric acid about 2 to 4 grams, 3 grams of the sodium salt of 4-hydroxyl pentanoic acid, the Class III agent alprazolam 3 mg, the Class IV agent zolpidem 10 mg, and the Class V agent gabapentin 300 mg prior to sleep.

### EXAMPLE 114

A patient with ARG takes the Class II agents the sodium salt of gamma-hydroxyl butyric acid about 2 to 4 grams and 4-hydroxyl pentanoic acid lactone 3 grams, the Class III agent alprazolam 3 mg, the Class IV agent zolpidem 10 mg, and the Class V agent gabapentin 300 mg prior to sleep.

### EXAMPLE 115

A patient with ARG takes the Class I agent red wine about 250-500 ml, the Class III agent about 2 mg alprazolam, the Class IV agent zolpidem about 10 mg, and the Class V agent gabapentin prior to sleep. About 6 hours later the patient takes the Class II agent the sodium salt of 4-hydroxyl pentanoic acid about 4 grams or 4-hydroxyl pentanoic acid lactone about 4 grams to obtain further stabilized sleep.

### EXAMPLE 116

A patient with ARG is treated with the Class IV agent zolpidem about 5 to 20 mg and the Class II agent 4-hydroxyl pentanoic acid lactone about 0.8 to 10 grams prior to sleep. Doses should be started low and increased slowly.

### EXAMPLE 117

A patient with ARG is treated with the Class IV agent zaleplon about 5 to 60 mg and the Class II agent 4-hydroxyl pentanoic acid lactone about 0.8 to 10 grams prior to sleep.

### EXAMPLE 118

A patient with ARG is treated with the Class III agent alprazolam about 0.25 to 4 mg and the Class II agent 4-hydroxyl pentanoic acid lactone about 0.8 to 10 grams prior to sleep.

### EXAMPLE 119

A patient with ARG is treated with the Class V agent gabapentin about 100 to 800 mg, the Class III agent alprazolam about 0.25 to 4 mg, the Class IV agent zolpidem about 5 to 30 mg, and the Class IV agent 4-hydroxyl pentanoic acid lactone about 0.8 to 10 grams prior to sleep.

### EXAMPLE 120

A patient with ARG is treated with the Class V agent gabapentin about 100 to 800 mg, the Class III agent alprazolam 0.25 to 4 mg, the Class IV agent zaleplon 5 to 60 mg, and the Class II agent 4-hydroxyl pentanoic acid lactone about 0.8 to 10 grams prior to sleep.

### EXAMPLE 121

A patient with ARG is treated with the Class IV agent zolpidem about 5 to 30 mg and the Class II agent the sodium salt of 4-hydroxyl pentanoic acid about 0.9 to 10 grams prior to sleep.

### EXAMPLE 122

A patient with ARG is treated with the Class IV agent zaleplon about 5 to 60 mg and the Class II agent the sodium salt of 4-hydroxyl pentanoic acid about 0.9 to 10 grams prior to sleep.

### EXAMPLE 123

A patient with ARG is treated with the Class III agent alprazolam about 0.25 to 4 mg and the Class II agent the sodium salt of 4-hydroxyl pentanoic acid about 0.9 to 10 grams prior to sleep.

### EXAMPLE 124

A patient with ARG is treated with the Class V agent gabapentin about 100 to 800 mg, the Class III agent alprazolam 0.25 to 4 mg, the Class IV agent zolpidem 5 to 30 mg, and the Class II agent the sodium salt of 4-hydroxyl pentanoic acid about 0.9 to 10 grams prior to sleep. Doses should be started low and increased as tolerated over weeks to months.

### EXAMPLE 125

A patient with ARG is treated with the Class V agent gabapentin about 100 to 800 mg, the Class III agent alprazolam 0.25 to 4 mg, the Class IV agent zaleplon 5 to 60 mg, and the Class II agent the sodium salt of 4-hydroxyl pentanoic acid about 0.9 to 10 grams prior to sleep.

### EXAMPLE 126

A patient with ARG is given the sodium salt of 4-hydroxyl pentanoic acid about 1 to 10 grams prior to sleep and a repeat dose about two to seven hours after sleep onset. In a preferred aspect, the first and second doses are about 3 to 7 grams. The second dose may be given about 4 hours after sleep onset or upon spontaneously awakening. Doses should be started low and increased slowly over weeks to months.

### EXAMPLE 127

A patient with ARG is given about 1 to 10 grams of 4-hydroxyl pentanoic acid lactone prior to sleep and a repeat dose about two to seven hours after sleep onset. In a preferred aspect, the first and second doses are about 3 to 7 grams. The second dose may be given about 4 hours after sleep onset or upon spontaneously awakening. Doses should be started low and slowly increased over weeks to months.

### EXAMPLE 128

A patient with ARG is given about 1 to 4 grams of the sodium salt of 4-hydroxyl pentanoic acid and about 1 to 4 grams of the sodium salt of 4-hydroxyl pentanoic acid prior to sleep. Doses should be started low and slowly increased as tolerated. One or more doses of the sodium salt of 4-hydroxyl pentanoic acid or 4-hydroxyl pentanoic acid lactone may be given later in the night.

### EXAMPLE 129

Most patients with ARG require moderate to high doses of one or more GABAnergic agents. However, on occasion a patient may be treated with low doses of multiple GABAnergic agents, such as the Class II agent the sodium salt of 4-hydroxyl pentanoic acid or 4-hydroxyl pentanoic acid lactone 0.1 to 1.5 grams, the Class IV agent zolpidem 1 to 5 mg, the Class III agent alprazolam 0.1 to 0.25 mg, and/or the Class V agent gabapentin 25 to 100 mg.

### EXAMPLE 130

A patient with ARG or insomnia is treated with the Class I agent red wine about 13% alcohol by volume 40 to 2000 ml, the Class III agent alprazolam about 2 mg, the Class IV agent zolpidem about 10 mg, the Class V agent gabapentin 300 mg, and the Class II agent the sodium salt of 4-hydroxyl pentanoic acid or 4-hydroxyl pentanoic acid lactone about 1 to 10 grams. Doses should be started low and increased over weeks to months.

### EXAMPLE 131

A patient who has side effects on a given dose of Class II GABAnergic agent may be treated with a lower dose of the Class II agent and increased dosages of other GABAnergic agents. A patient who has side effects due to 4-hydroxyl pentanoic acid, salts thereof, or 4-hydroxyl pentanoic acid lactone on 4 grams of the sodium salt of 4-hydroxyl pentanoic acid and 2 mg alprazolam may instead be treated with a lesser dose of the sodium salt of 4-hydroxyl pentanoic acid such as 2 grams and the alprazolam may be increased in dose and/or other GABAnergic agents such as zolpidem or gabapentin may be added to the regimen. Given the wide variety of GABAnergic agents available, numerous other regimens are possible to decrease the dose of Class II agents.

What is claimed is:

1. A method of treating patients with acquired resistance to GABAnergic agents (ARG) comprising administering therapeutically effective amounts of two or more GABAnergic agents prior to sleep to a patient in need of such treatment where each agent is independently selected from Class II GABAnergic agents, in which Class II GABAnergic agents are gamma-hydroxyl butyric acid or salts thereof; gamma-butyrolactone; 1,4-butanediol; 4-hydroxyl pentanoic acid or salts thereof; 4-hydroxyl pentanoic acid lactone; or combinations thereof; Class III GABAnergic agents, which are benzodiazepines, Class IV GABAnergic agents, which bind subunits or configurations of the GABA receptor more specifically than benzodiazepines, Class V GABAnergic agents, which are structural analogs of gamma-amino butyric acid which do not bind the GABA receptor, and Class VI GABAnergic agents, which are barbiturates.

2. A method of treating patients according to claim 1 in which the Class II agent is the sodium salt of gamma-hydroxyl butyric acid.

3. A method of treating patients according to claim 1 in which the Class III GABAnergic agent comprises alprazolam, clonazepam, diazepam, lorazepam, clorazepate, oxazepam, flurazepam, estalozam, triazolam, chlordiazepoxide, oxazepam, prazepam, quazepam, temazepam, nitrazepam, or combinations thereof.

4. A method according to claim 3 in which the Class III agent is alprazolam, clonazepam, or combinations thereof.

5. A method according to claim 1 in which the Class IV GABAnergic agent is zolpidem, zaleplon, zopiclone, eszopiclone, or combinations thereof.

6. A method according to claim 5 in which the Class IV GABAnergic agent is zolpidem.

7. A method according to claim 1 in which the Class V GABAnergic agent is gabapentin.

8. A method according to claim 1 in which a Class II agent is combined with a class III agent.

9. A method according to claim 9 in which the Class III agent is alprazolam or clonazepam.

10. A method according to claim 1 in which a Class II agent is combined with a Class IV agent and administered prior to sleep; where the Class II agent is gamma-hydroxyl butyric acid or a pharmaceutically acceptable salt thereof; GBL; 1,4-butanediol; 4-hydroxyl pentanoic acid or a pharmaceutically acceptable salt thereof; 4-hydroxyl pentanoic acid lactone; or combinations thereof; and the class IV agent is zolpidem.

11. A method according to claim 1 in which a Class II agent and a Class III agent are administered prior to sleep; furthermore the Class II agent is gamma-hydroxyl butyric acid or a pharmaceutically acceptable salt thereof; GBL; 1,4-butanediol; 4-hydroxyl pentanoic acid or a pharmaceutically acceptable salt thereof; 4-hydroxyl pentanoic acid lactone; or combinations thereof; and the class III agent is alprazolam or clonazepam or combinations thereof.

12. A method according to claim 1 in which a Class II agent, a Class III agent, and a Class IV agent are administered prior to sleep; furthermore the Class II agent is gamma-hydroxyl butyric acid or a pharmaceutically acceptable salt thereof; GBL; 1,4-butanediol; 4-hydroxyl pentanoic acid or a pharmaceutically acceptable salt thereof; 4-hydroxyl pentanoic acid lactone; or combinations thereof; the class III agent is alprazolam or clonazepam or combinations thereof; and furthermore the Class IV agent is zolpidem, zaleplon, zopliclone, eszoplicone, or combinations thereof.

13. A method of treating patients with acquired resistance to GABAnergic agents (ARG) according to claim 1 in which a Class II agent and a Class III agent and a Class IV agent and a Class V agent are administered prior to sleep; the Class II agent is gamma-hydroxyl butyric acid or a pharmaceutically acceptable salt thereof; GBL; 1,4-butanediol; 4-hydroxyl pentanoic acid or a pharmaceutically acceptable salt thereof; 4-hydroxyl pentanoic acid lactone; or combinations thereof; the Class III agent is alprazolam or clonazepam, or combinations thereof; the Class IV agent is zolpidem, zaleplon, zopliclone, eszoplicone, or combinations thereof; the Class V agent is gabapentin.

14. A method according to claim 13 where the Class IV agent is zolpidem.

15. A method of treating patients with ARG according to claim 1 in which the Class III agent alprazolam, the Class IV agent zolpidem, the Class V agent gabapentin, and the Class II agent the sodium salt of gamma-hydroxyl butyric acid are administered prior to sleep.

16. An aqueous, pharmaceutical composition of the sodium salt of gamma-hydroxyl butyric acid, wherein the composition contains about 10 to about 750 mg/ml of NaGHB, a buffering agent to maintain the pH of the solution at about 6 to 9, a preservative, a coloring agent, and at least one flavoring agent; furthermore, the preservative is sodium benzoate, methyl paraben, ethyl paraben, or combinations thereof, the coloring agent is FD & C Blue No. 1, FD & C Blue No. 2, FD & C Green 1, FD & C Green No. 2, FD & C Green No. 3, FD & C Orange 1, FD & C Red No. 1, FD & C Red No. 2, FD & C Red No. 3, FD & C Red No. 4, FD & C Red No. 32, FD & C Red No. 40, FD & C Yellow No. 1, FD & C Yellow No. 3, FD & C Yellow No. 4, FD & C Yellow No. 5, FD & C Yellow No. 6 or combinations thereof, and the flavoring agent is menthol or menthone.

17. A pharmaceutical composition according to claim 16 which contains about 10 to 100 mg/ml NaGHB.

18. An aqueous, pharmaceutical composition of gamma-butyryl lactone (GBL), wherein the composition contains about 10 to about 900 mg/ml of GBL, a buffering agent to maintain the pH of the solution at 2 to 9, a coloring agent, and at least one flavoring agent; furthermore, the coloring agent is FD & C Blue No. 1, FD & C Blue No. 2, FD & C Green 1, FD & C Green No. 2, FD & C Green No. 3, FD & C Orange 1, FD & C Red No. 1, FD & C Red No. 2, FD & C Red No. 3, FD & C Red No. 4, FD & C Red No. 32, FD & C Red No. 40, FD & C Yellow No. 1, FD & C Yellow No. 3, FD & C Yellow No. 4, FD & C Yellow No. 5, FD & C Yellow No. 6 or combinations thereof, and the flavoring agent is menthol or menthone.

19. A pharmaceutical composition according to claim 18 which further comprises a preservative; furthermore, the preservative is sodium benzoate, methyl paraben, ethyl paraben, propyl paraben, or combinations thereof.

20. A method of treating acquired resistance to GABAnergic agents comprising administering a therapeutic amount of gamma-hydroxyl butyric acid or a pharmaceutically acceptable salt thereof; GBL; 1,4-butanediol; 4-hydroxyl pentanoic acid or a pharmaceutically acceptable salt thereof; or 4-hydroxyl pentanoic acid lactone to a patient in need of such treatment.
